SEQUENCE LISTING (3 sequences)

SEQ ID NO: 1 - DNA, 32 bp, synthetic construct
  source 1..32: mol_type = other DNA
  modified_base 1: mod_base = OTHER; note = 5' ATTO488K conjugated
  modified_base 32: mod_base = OTHER; note = 3' ATTO647NN conjugated
ttagttgtaaaccatgatgtgctgctaatgta

SEQ ID NO: 2 - DNA, 34 bp, synthetic construct
  source 1..34: mol_type = other DNA
  modified_base 1: mod_base = OTHER; note = 5' Alexa546N conjugated
  modified_base 34: mod_base = OTHER; note = 3' ATTO647NK conjugated
ttagtaacccctatcacgattagcattaaatgta

SEQ ID NO: 3 - DNA, 36 bp, synthetic construct
  source 1..36: mol_type = other DNA
  modified_base 1: mod_base = OTHER; note = 5' ATTO488N conjugated
  modified_base 36: mod_base = OTHER; note = 3' ATTO565N conjugated
acttagtcgcattattactcacggtacgaatgtatc

(19) United States
(12) Patent Application Publication
(10) Pub. No.: US 2026/0235515 A1
(43) Pub. Date: Aug. 13, 2026

(54) METHOD OF IMAGING BIOMOLECULES

(71) Applicant: Ramot at Tel-Aviv University Ltd., Tel-Aviv (IL)

(72) Inventors: Yuval EBENSTEIN, Tel Aviv (IL); Sayan MONDAL, Tel Aviv (IL); Jonathan JEFFET, Tel Aviv (IL); Jasline DEEK, Tel Aviv (IL)

(73) Assignee: Ramot at Tel-Aviv University Ltd., Tel-Aviv (IL)

(21) Appl. No.: 19/470,994

(22) PCT Filed: Mar. 29, 2024
(86) PCT No.: PCT/IL2024/050330

§ 371 Date: Sep. 30, 2025

(30) Foreign Application Priority Data
Mar. 30, 2023 (GB) 2304755.8

(51) Int. Cl. G01N 21/64; G01N 21/17; G01N 33/68

(52) U.S. Cl. CPC G01N 21/6428; G01N 21/17; G01N 33/6803; G01N 2021/174; G01N 2400/00; G01N 2405/00; G01N 2458/00; G01N 2800/00

(57) ABSTRACT

Identification of molecular species in a sample based unique fluorescent labeling, light dispersion, and image processing. The molecular species identification methods can be used for diagnosing diseases.

### RELATED APPLICATIONS

This application claims the benefit of priority of Great Britain Patent Application No. 2304755.8 filed on Mar. 30, 2023, the contents of which are incorporated herein by reference in their entirety.

### SEQUENCE LISTING STATEMENT

The XML file, entitled 99037 Sequence Listing.xml, created on Mar. 29, 2024, comprising 6,733 bytes, submitted concurrently with the filing of this application is incorporated herein by reference.

### FIELD AND BACKGROUND OF THE INVENTION

The present invention, in some embodiments thereof, relates to methods of identifying biomolecules.

The multiplexed detection of biomolecules plays an important role in clinical diagnostics, discovery, and basic science. This requires the ability to both encode substrates associated with specific biomolecule targets, and also to associate a detectable signal to the biomolecule target being quantified. For multiplexed assays, it is common to use functionalized substrates, planar or particle-based, to capture and quantify targets. In the case of particle-based multiplexed assays, each particle is functionalized with a probe that captures a specific target, and encoded for identification during analysis. In order to quantify the amount of target captured on a particle, a suitable labeling scheme is typically used to provide a measurable signal associated with the target. One class of molecules that is particularly challenging to quantify due to limitations with existing approaches to labeling is microRNA (miRNA).

miRNAs are short non-coding RNAs that mediate protein translation and are known to be dysregulated in diseases including diabetes, Alzheimer's, and cancer. With greater stability and predictive value than mRNA, this relatively small class of biomolecules has become increasingly important in disease diagnosis and prognosis. However, the sequence homology, wide range of abundance, and common secondary structures of miRNAs have complicated efforts to develop accurate, unbiased quantification techniques. Applications in the discovery and clinical fields require high-throughput processing, large coding libraries for multiplexed analysis, and the flexibility to develop custom assays. Microarray approaches provide high sensitivity and multiplexing capacity, but their low-throughput, complexity, and fixed design make them less than ideal for use in a clinical setting. PCR-based strategies suffer from similar throughput issues, require lengthy optimization for multiplexing, and are only semi-quantitative. Existing bead-based systems provide a high sample throughput (>100 samples per day), but with reduced sensitivity, dynamic range, and multiplexing capacities. Therefore, there is a need for improved methods for detecting and quantifying nucleic acids, such as, miRNA.

The multiplexed detection of miRNAs, or any other biomolecules requires the ability to encode a substrate associated with each. There are two broad classes of technologies used for multiplexing—planar arrays and suspension (particle-based) arrays, both of which have application-specific advantages. While planar arrays rely strictly on positional encoding, suspension arrays have utilized a great number of encoding schemes that can be classified as spectrometric, graphical, electronic, or physical.

Spectrometric encoding encompasses any scheme that relies on the use of specific wavelengths of light or radiation (including fluorophores, chromophores, photonic structures, or Raman tags) to identify a species. Fluorescence-encoded microbeads can be rapidly processed using conventional flow-cytometry (or on fiber-optic arrays), making them a popular platform for multiplexing. Most spectrometric encoding methods rely on the encapsulation of detectable entities for encoding, which can be very challenging depending on the substrate used. A more robust and generally-applicable encoding method is needed to enable rapid, universal encoding of substrates for multiplexed detection.

Background Art includes Jeffet et al., Biophysical Reports 1, 100013, Sep. 8, 2021; Zhang et al., Chem. Sci., 2020, 11, 3812.

### SUMMARY OF THE INVENTION

According to an aspect of some embodiments of the present invention there is provided a method of identifying a plurality of molecular species in a sample. The method comprises: for each of at least two of the molecular species, labeling two locations on the species with respective two fluorophores having different emission wavelengths, wherein a combination of the fluorophores and/or a spatial distance between the fluorophores is unique for each of the at least two molecular species to generate at least two uniquely labeled molecular species. The method further comprises dispersing light emitted from the fluorophores onto an imager to generate an image of fluorophore emissions from the at least two uniquely labeled molecular species, wherein for any pair of emission wavelengths, a distance between fluorophore emissions corresponding to the pair is unique among all other pairs; and correlating distances between fluorophore emissions in the image to the at least two of the molecular species in the sample, thereby identifying a plurality of molecular species in the sample.

According to an aspect of some embodiments of the present invention there is provided a method of diagnosing a disease of a subject comprising identifying a plurality of molecular species in a sample of the subject. The method comprises: for each of at least two of the molecular species, labeling two locations on the species with respective two fluorophores having different emission wavelengths, wherein a combination of the fluorophores and/or a spatial distance between the fluorophores is unique for each of the at least two molecular species to generate at least two uniquely labeled molecular species. The method further comprises dispersing light emitted from the fluorophores onto an imager to generate an image of fluorophore emissions from the at least two uniquely labeled molecular species, wherein for any pair of emission wavelengths, a distance between fluorophore emissions corresponding to the pair is unique among all other pairs; and identifying the at least two of the molecular species by correlating distances between fluorophore emissions in the image to the at least two of the molecular species in the sample.

According to some embodiments of the present invention a presence and/or an amount of the at least two molecular species is indicative of the disease.

According to some embodiments of the invention, the combination of the fluorophores is unique among all other fluorophore-labeled molecular species in the sample.

According to some embodiments of the invention, the dispersing light from the fluorophores onto the imager generates a single image of fluorophore emissions from the at least two uniquely labeled molecular species.

According to some embodiments of the invention, the fluorophores emit light by fluorescence emission.

According to some embodiments of the invention, the fluorophores emit light by phosphoresce emission.

According to some embodiments of the invention, the fluorophores are arranged along a direction over the molecular species, and wherein the dispersing is one-dimensional along the direction.

According to some embodiments of the invention, the fluorophores are arranged along a first direction over the molecular species, and wherein the dispersing is one-dimensional along a second direction, different from the first direction.

According to some embodiments of the invention, the first and the second directions are generally orthogonal to each other.

According to some embodiments of the invention, the dispersing is two-dimensional.

According to some embodiments of the invention, at least one molecular species is labeled by at least three spaced apart locations on the species with respective at least three different fluorophores characterized by different emission wavelengths.

According to some embodiments of the invention, the image is a spectral image.

According to some embodiments of the invention, the dispersing comprises non-linear dispersing.

According to some embodiments of the invention, the dispersing comprises linear dispersing.

According to some embodiments of the invention, the dispersing is by a dispersive element selected from the group consisting of a prism, a grating, and a grism.

According to some embodiments of the invention, a combination of the fluorophores is unique for each of the at least two molecular species to generate at least two uniquely labeled molecular species.

According to some embodiments of the invention, a spatial separation between the locations is at most 100 nm, or at most 50 nm, or at most 25 nm, or at most 10 nm.

According to some embodiments of the invention, a distance between the fluorophores is unique for each of the at least two molecular species to generate at least two uniquely labeled molecular species.

According to some embodiments of the invention, the distance is greater than 250 nm.

According to some embodiments of the invention, the imager is a component in an imaging system characterized by a diffraction limit, wherein a largest among spatial separations between the locations is less than the diffraction limit, and wherein a smallest among the distances between fluorophore emissions is larger than the diffraction limit.

According to some embodiments of the invention, the method further comprises analyzing intensity distribution of the fluorophore emissions in the image and correlating the intensity distribution to the at least two of the molecular species in the sample.

According to some embodiments of the invention, the method further comprises orientating the at least two uniquely labeled molecular species with respect to one another.

According to some embodiments of the invention, the method further comprises orientating the at least two uniquely labeled molecular species along a direction.

According to some embodiments of the invention, the method further comprises immobilizing the at least two uniquely labeled molecular species on a solid surface.

According to some embodiments of the invention, the immobilizing comprises selectively immobilizing the at least two uniquely labeled molecular species on a solid surface whilst not immobilizing non-labeled molecular species on the solid surface.

According to some embodiments of the invention, the immobilizing is effected using an antibody which is attached to the solid surface.

According to some embodiments of the invention, a distance between the two locations is identical for the plurality of molecular species.

According to some embodiments of the invention, the molecular species are selected from the group consisting of a polypeptide, a polynucleotide, a lipid and a carbohydrate.

According to some embodiments of the invention, the molecular species is a polynucleotide.

According to some embodiments of the invention, the polynucleotide is an RNA.

According to some embodiments of the invention, the RNA is a miRNA.

According to some embodiments of the invention, the labeling comprises hybridizing a probe to the polynucleotide, the probe being labeled with the two fluorophores.

According to some embodiments of the invention, the probe is a DNA probe.

According to some embodiments of the invention, the at least two molecular species comprises at least 5 molecular species.

According to some embodiments of the invention, the method further comprises quantifying the at least two molecular species.

According to some embodiments of the invention, the identifying is at a single molecule level.

According to some embodiments of the invention, the sample is a cellular sample.

According to an aspect of some embodiments of the present invention there is provided a method of identifying a plurality of molecular species in an image containing a plurality of fluorophore emissions collected from uniquely labeled molecular species. The method comprises: classifying pairs of fluorophore emissions in the image according to intra-pair distances, to provide a plurality of classes; accessing a computer readable medium storing a mapping between intra-pair distances and labeled molecular species; and using the mapping for identifying molecular species of at least one class based on a respective intra-pair distance of the class.

According to some embodiments of the invention, the intra-pair distances correspond to the difference in emission wavelength of at least two fluorophores which label the molecular species and/or a spatial distance between the at least two fluorophores on the molecular species.

According to an aspect of some embodiments of the present invention there is provided a computer software product, comprising a computer-readable medium in which program instructions are stored, which instructions, when read by a data processor, cause the data processor to receive an image containing a plurality of fluorophore emissions and to execute the method described herein.

According to an aspect of some embodiments of the present invention there is provided a method of identifying a plurality of molecular species in a sample. The method comprises: for each of the molecular species, labeling a plurality of locations along a first direction on the species with a sequence of fluorophores, wherein a spatial distribution of the fluorophores along the first direction is unique for each of the at least two molecular species to generate at least two uniquely labeled molecular species. The method further comprises dispersing light emitted from the fluorophores along a second direction onto an imager to generate an image of fluorophore emissions from the at least two uniquely labeled molecular species, wherein the first direction is different from the second direction; identifying different two-dimensional patterns of fluorophore emissions in the image; and correlating the identified patterns to at least two of the molecular species in the sample, thereby identifying a plurality of molecular species in the sample.

According to some embodiments of the invention, for at least one of the species, the sequence includes repeats.

According to some embodiments of the invention, for at least one of the species, the sequence is non-repetitive.

According to some embodiments of the invention, a distance between adjacent fluorophores in the sequence is greater than 250 nm.

According to some embodiments of the invention, the at least two different species are labeled by identical sequences of fluorophores but using different spatial distributions.

According to some embodiments of the invention, a spatial separation between the locations is at most 100 nm, or at most 50 nm, or at most 25 nm, or at most 10 nm.

According to some embodiments of the invention, any two different species are labeled by different sequences of fluorophores.

According to some embodiments of the invention, the identifying the two-dimensional patterns comprises calculating a Point Spread Function (PSF).

According to some embodiments of the invention, the identifying and the correlating is by a machine learning procedure.

According to some embodiments of the invention, the directions are generally orthogonal to each other.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D provide an overview of the identification methods described herein, according to embodiments of the invention. A) All RNAs are extracted from plasma samples and only the selected miR targets are selectively hybridized with fluorescent dye-pair labeled DNA reporter probes. B) The hybridized target miR: reporter DNA complexes are selectively captured on a glass surface by an Anti-DNA-RNA hybrid [S9.6] antibody while all excess probes and molecules are washed away. The captured targets are then imaged using spectral imaging microscope with a single frame capturing all fluorescent probes in the field of view (FOV). C) The spectral images are then scanned for detecting all single-molecule miRs across multiple FOVs covering the detection area of the slide. D) the miRs are classified according to the distinctive spectral signature of each reporter's dye-pair, which can be simulated (left dashed rectangle) with varying gaussian noise distributions (noise variance is depicted on top) to a high extent of resemblance with the real data (right yellow dashed rectangle). Finally, the classified miRs are summed to produce an absolute expression profile which can be used for diagnosis. Data shown in C and D is taken from the synthetic miR 1:1:1 mixture sample as described in text and methods. Scale bars in C are 10 m.

FIG. 2 is a simulation of 11 reporter probes PSFs. The calculated distances between the peaks are presented at the bottom. The dye-pair names are, from left to right: AF568-AF647, AF488-Cy2, ATT0520-AF594, AF546-AF660, AR594-AF750, AF488-AF594, 4F514-ATT0700, ATT0520-AF660, ATT0520-ATTO490LS, AF488-AF660, AF488-ATT0700.

FIGS. 3A-B. CLL diagnosis results according to two miR targets ratio. A) counts per FOV for the two miR targets detected in the CLL diagnosis experiment. B) The ratio between miR-155-5p and miR-15b-5p for each sample showing a distinct signature for CLL in the ratio of the two miR targets, as expected from previous studies.

FIGS. 4A-B. Fluorescently labelled single stranded DNA (ssDNA) capture probe does not non-specifically bind to Peg and Peg-biotin passivated surface. 100 ul of 100 pM ssDNA labelled with Alexa 647 (ssDNA-A647) was incubated on peg-biotinylated (Peg:Peg-Biotin=100:1) glass surface for one hour, then washed 3 times with 100 ul PBS before imaging, and a field of view (FOV) with (A) 633 nm and with (B) 561 nm laser in shown. The FOV with 633 nm excitation shows that the ssDNA-A647 probe dies not stick on the passivated surface, and get washed effectively with 3× gentle washing. The bright particles in (A) are not ssDNA-A647, and are other impurities seen in (B) with 561 nm laser excitation.

FIGS. 5A-B. ssDNA capture probes partially bind to S9.6 anti-DNA:RNA hybrid antibody. 100 ul of 100 pM ssDNA labelled with Alexa 647 (ssDNA-A647) was incubated on peg-biotinylated (Peg:Peg-Biotin=100:1) glass surface with immobilized S9.6 antibody for one hour, then washed the 3 times with 100 ul PBS before imaging, and a field of view (FOV) with (A) 633 nm and with (B) 561 nm laser in shown. The FOV with 633 nm excitation shows that the ssDNA-A647 probes indeed partially bind to S9.6-immobilized and PEG passivated surface. The bright particles in (A) are ssDNA capture probes, whereas very few bright spots seen in (B) with 561 nm laser excitation confirm that the single particles seen in (A) are indeed ssDNA-A647 capture probes.

FIGS. 6A-B. S9.6 anti-DNA:RNA hybrid antibody does not capture single stranded microRNAs. 100 ul of 100 pM synthetic miR (hsa-mir-15b-5p) labelled with Alexa 488 (miR-A488) was incubated on peg-biotinylated (Peg:Peg-Biotin=100:1) glass surface with immobilized S9.6 antibody for one hour, then washed the 3 times with 100 ul PBS before imaging, and a field of view (FOV) with (A) 488 nm and with (B) 561 nm laser in shown. The FOV with 633 nm excitation shows that the synthetic miR-A488 have small affinity for binding to immobilized S9.6 antibody and PEG passivated surface.

FIGS. 7A-B. S9.6 anti-DNA:RNA hybrid antibody specifically captures DNA:RNA duplex, and does not catch either ss-miR or ss-DNA capture probes in presence of DNA:RNA duplexes. CoCoS image with single exposure of 800 ms with both 488 and 633 nm excitation, and at RPA 175 for optimal detection of dispersed emission. ssDNA-A647 capture probe is in (A) one time excess, and (B) seven times excess to DNA:RNA hybrids while incubating on immobilized S9.6 antibody, and then washed 3× with PBS before imaging. The DNA:RNA hybrids are detected as doubly labelled single molecule as ss-DNA capture probe is labelled Alexa 647, and the target hsa-miR-15b-5p is labelled with A488. (see Experimental methods for more details) In both (A) and (B) more than 98% detectable signal are from duplex (two dots separated by 12 pixel), and single labelled either ss-DNA or hsa-miR-15b-5p is nor observed.

FIG. 8. PSF simulations. 11 dye-pairs with distinct PSFs were simulated in Matlab according to the dyes' spectra and the dispersion profile of CoCoS with RPA=177.5. Each row corresponds to simulated gaussian noise (G.N) added over the same PSF data as in the first row. The G.N is distributed around 0.3 mean and with variance as depicted to the left (corresponding roughly to SNR of about 25, 7 and 1.7, from top to bottom). Poisson distributed noise was added to all noise cases. As can be seen the gaussian noise which corresponds to the fluorescence background and pixel readout noises is the main limiting factor for efficient classification of multiple miR targets. From left to right: ATTO520-AF594, AF546-AF660, AF594-AF750, AF488-AF594, AF514-ATTO700, ATTO520-AF660, ATTO520-ATTO490LS, AF488-AF660, and AF488-ATTO700.

FIGS. 9A-D. Resolving multi-color barcodes. A) Illustration of a linear four-color barcode detected with two acquisition pipelines. Top, standard color imaging overlaying four images acquired with dedicated emission filters to resolve the ground truth (GT). Bottom, color image reconstruction with DeepQR: a single spectral image acquired by dispersing fluorescence emission with two direct-vision prisms is converted to a multi-color prediction image (Pred) by a deep neural network (DNN) U-Net. B) Theoretical emission spectra of the six potential barcode dyes through DeepQR's three spectral windows. The three excitation lasers used for six-color detection are displayed as solid lines. Colored patches indicate the multi-band emission filter channels, showing that DeepQR allows resolving the overlapping spectra of two different dyes in each spectral channel. C) Simulation of a single barcode with six dyes dispersed to create a spectral QR barcode. Left: the non-dispersed labeled barcode, Right: the dispersed barcode allowing to interpret its composition with its unique spectral PSF. D) Simulated examples of various randomly generated 6-color barcodes. Dye names are displayed to the left of each label. Using six dyes per barcode allows to tag up to 18,750 unique RNA targets.

FIGS. 10A-G. NanoString's inflammation panel barcode classification and gene expression analysis using DeepQR for Ulcerative Colitis detection. A) NanoString's dyes spectra overlayed with the CoCoS system's single three-band emission filter. The three excitation lasers used for four-color detection are displayed as solid lines. Colored patches indicate the multi-band emission filter channels, showing that DeepQR allows resolving the overlapping spectra of Cy3 and AF594 using a single spectral channel.B) Examples of barcode classification using DeepQR prediction. Leftmost two columns:simulated (Sim) and experimental (Disp) dispersed single frame acquisition excited by all three lasers, capturing the barcode information in a 2D single color barcode (dispersed color legend presented on the top). Prediction column (Pred): False color representation of the four-color U-Net prediction, according to the dispersed images. Rightmost column (GT): False colored ground-truth overlays of sequential four-channel acquisition with dedicated laser excitation and emission filter per channel and no dispersion (RPA 180°). The name of the corresponding target gene is shown next to each barcode. C) Unsupervised clustering and heatmap representation of the mean-subtracted normalized log 2 barcode expression values, comparing four samples of healthy individuals and patients with ulcerative colitis (UC) between the three detection methods: DeepQR prediction (Pred), ground-truth (GT) and the commercial NanoString nCounter system. D) Log 2 of normalized barcode count distributions across all samples and readout methods. E) Multidimensional scaling (MDS) plot showing the classification of healthy and UC samples between the three methods. F) Barcode distribution comparison of the 25 most abundant barcodes detected in the three methods. G) Set comparison between matched pairs of eligibly read barcodes in GT and Pred (termed common barcodes). The area of circles corresponds with the number of barcodes in each set (GT or Pred) and the ratio of identical barcodes is presented at the center.

FIGS. 11A-C. The miRACLE scheme: A) Total RNA is extracted from plasma samples and only the selected miR targets are specifically hybridized with DNA capture-reporter probes. Each reporter has a unique fluorophore-pair combination (fluorophores names are displayed to the left, Alexa Fluor—AF). B) The hybridized target miR: reporter DNA complexes are selectively captured on a glass surface by an Anti-DNA-RNA hybrid [S9.6] antibody while all excess probes and molecules are washed away. The glass antibody surfaces are prepared in-house using biotin-streptavidin binding and polyethylene glycol (PEG) passivation. C) The captured targets are then imaged using a compact spectral imaging microscope module (CoCoS), which allows capturing all fluorescent probes in the FOV with a single frame (top). The miRs are classified according to the distinctive spectral signature of each reporter's fluorophore-pair (the fluorophores' identities are depicted at the bottom of each colored box on the left), which can be simulated (left) with varying Gaussian noise distributions (induced Gaussian noise variance is depicted at the bottom) to a high extent of resemblance with the real data (right). The expected distance between peaks is displayed to the right. All crops are presented with the same brightness and contrast settings.

FIGS. 12A-D. Automated multiplexed miR detection and classification at single-molecule resolution. A) Before detecting and classifying the probe's PSFs, three steps of image pre-processing are performed. First, using a multi-FOV stack we calculate and subtract the pixel-wise median value from the raw images to remove the constant excitation background. Next, we further process the images to enhance the signal by using the deep learning based Noise2Void (N2V) algorithm. B) The denoised images are inputted to the ThunderSTORM (TS) localization plugin to detect all Gaussian spots and crop 24×10 pixels rectangles around each Gaussian spot for further analysis (example crops are on the right). C) Single miR type samples are used for generating a training set for the automatic classifier. A small subset of about 1000 crops are visually inspected by a user in V-TIMDER, a dedicated graphical user interface. The user visually tags each crop as a valid miR PSF or as noise. D) The tagged crops gathered from three single miR type samples are computationally mixed and used for training a machine-learning classifier model using support vector machines (SVM) combined with principal component analysis (PCA). The classifier is used to automatically classify mixed miR samples according to their PSFs: three different miR types (orange, green and blue) or noise (yellow). Scale bars on FOV images in B and D are 30 m.

FIGS. 13A-D. Experimental classification results. A) Absolute count distributions of single miR type samples automatically classified from 241 (miR 15b), 324 (miR 155) and 303 (miR 126) FOVs. The distributions show minimal cross-assignment of the classifier between miR types. Each panel shows the classified miR type for all crops taken from a sample containing only the type specified in the legend. B) Confusion matrix for a mixed 1:1:1 miR sample (counted in 10 FOVs). The matrix compares the visually determined label to the automated classifier prediction. The matrix is predominately diagonal showing most classifier errors to be false noise classification and very little cross assignment between miR types. The precision and recall metrics are summarized for each class next to the confusion matrix. C) Precision and recall of each miR type as a function of the classification probability threshold. The area under the precision recall curve (AUC-PR) for the classification of each miR type is indicated in parentheses. Circles correspond to the precision and recall calculated from the confusion matrix in B. D) Determining the ratio of miR targets in an “unknown” synthetic mixed sample. The three synthetic miRs were mixed in a 2:5:3 ratio and were classified without supervision, and systematic errors were corrected by using the confusion matrix in panel B. We were able to correctly resolve the underlying ratios of miRs (circles, total of 63,493 classified crops in 630 FOVs). The error bars represent uncertainty of a 95% confidence interval. Diamonds represent the calculated ratio for a small subset of visually labeled crops (519 classified crops in 5 FOVs, about 0.82% of the full dataset).

FIG. 14 is a schematic illustration of a system which can be employed for executing a method that identifies a plurality of molecular species in a sample, according to some embodiments of the present invention.

FIGS. 15A-B—Fluorescently labeled single-stranded DNA (ssDNA) capture probe does not non-specifically bind to Peg and Peg-biotin passivated surfaces. 100 ul of 100 pM ssDNA labeled with Alexa 647 (ssDNA-A647) was incubated on peg-biotinylated (Peg:Peg-Biotin=100:1) glass surface for one hour, then washed 3 times with 100 ul PBS before imaging, and a field of view (FOV) with (A) 633 nm and with (B) 561 nm laser is shown. The FOV with 633 nm excitation shows that the ssDNA-A647 probe does not stick on the passivated surface and gets washed effectively with 3× gentle washing. The bright particles in (A) are not ssDNA-A647, and are other impurities seen in (B) with 561 nm laser excitation.

FIGS. 16A-B—ssDNA capture probes partially bind to S9.6 anti-DNA:RNA hybrid antibody. 100 ul of 100 pM ssDNA labeled with Alexa 647 (ssDNA-A647) was incubated on peg-biotinylated (Peg:Peg-Biotin=100:1) glass surface with immobilized S9.6 antibody for one hour, then washed 3 times with 100 ul PBS before imaging, and a field of view (FOV) with (A) 633 nm and with (B) 561 nm laser is shown. The FOV with 633 nm excitation shows that the ssDNA-A647 probes indeed partially bind to S9.6-immobilized and PEG passivated surfaces. The bright particles in (A) are ssDNA capture probes, whereas very few bright spots seen in (B) with 561 nm laser excitation confirm that the single particles seen in (A) are indeed ssDNA-A647 capture probes.

FIGS. 17A-B. S9.6 anti-DNA:RNA hybrid antibody does not capture single-stranded microRNAs. 100 ul of 100 pM synthetic miR (hsa-mir-15b-5p) labeled with Alexa 488 (miR-A488) was incubated on peg-biotinylated (Peg:Peg-Biotin=100:1) glass surface with immobilized S9.6 antibody for one hour, then washed the 3 times with 100 ul PBS before imaging, and a field of view (FOV) with (A) 488 nm and with (B) 561 nm laser is shown. The FOV with 633 nm excitation shows that the synthetic miR-A488 has a small affinity for binding to immobilized S9.6 antibody and PEG passivated surface.

FIGS. 18A-B. S9.6 anti-DNA:RNA hybrid antibody specifically captures DNA:RNA duplex, and does not catch either ss-miR or ss-DNA capture probes in presence of DNA:RNA duplexes. CoCoS image with a single exposure of 800 ms with both 488 and 633 nm excitation, and at RPA 175 for optimal detection of dispersed emission. ssDNA-A647 capture probe is in (A) one time excess, and (B) seven times excess to DNA:RNA hybrids while incubating on immobilized S9.6 antibody, and then washed 3× with PBS before imaging. The DNA:RNA hybrids are detected as doubly labeled single molecules as ss-DNA capture probe is labeled with Alexa 647, and the target hsa-miR-15b-5p is labeled with AF488. (see Experimental methods for more details) In both (A) and (B) more than 98% detectable signal originates from duplex (two blobs separated by 12 pixels), and singly labeled ss-DNA or hsa-miR-15b-5p are not observed.

FIG. 19. Theoretical emission spectra of the four fluorophores used for the three probes design. Colored patches indicate the multi-band emission filter channels (Table 5). The three lasers used for exciting the four fluorophores are displayed as solid vertical lines. The plot is stretched according to the non-linear dispersion curve of the optical system (FIG. 20), showing the theoretical pixel displacement (bottom X-axis) of each wavelength (top X-axis) in the fluorophores' spectra.

FIG. 20. Dispersion curve of the CoCoS setup using relative prism angle (RPA) of 177.5. The dispersion curve was experimentally extracted as previously described in ref 1. The colored circles correspond to the maximal emission wavelengths of the fluorophores used for designing the three probes used in the experiment, while the colored patches stand for the multi-band filter's transmission channels.

FIG. 21. Same curve as in FIG. 20 with maximal emission wavelengths of all fluorophores used for simulating PSFs of optional combinations for future probes.

FIG. 22. PSF simulations with varying noise distributions. 11 fluorophore-pairs with distinct PSFs were simulated in Matlab according to the fluorophores' spectra and the dispersion profile of CoCoS with RPA=177.5. Each row corresponds to simulated Gaussian noise (G.N) added over the same PSF data as in the first row. The G.N is distributed around 0.3 mean and with variance as depicted to the left (corresponding roughly to SNR of about 25, 7 and 1.7, from top to bottom). Poisson distributed noise was added to all noise cases. As can be seen, the Gaussian noise which corresponds to the fluorescence background and pixel readout noises is the main limiting factor for efficient classification of multiple miR targets. From left to right: AF568-AF647, ATTO520-AF594, AF546=AF5660, AF594-AF750, AF488-AF594, AF514-ATTO700, ATTO520-AF660, ATTO520-ATTO490LS, AF488-AF660, AF488-ATTO700.

FIG. 23. V-TIMDER visual explanation. Left, the V-TIMDER GUI in “mixture” mode explained on a miR-126 PSF in normal context crop size (24×10 pixels). In “binary” mode the miR type toggle is removed. Right, examples for a large context (48×20 pixels) crop of miR-15b PSF (top), extra-large context (240×100 pixels) crop of miR-155 (center), and large context of a single spot, noise crop (bottom).

FIG. 24. Mixtures' absolute counts distributions. The left y-axis represents the total number of counts produced by the classifier whereas the right y-axis represents the number of crops visually classified by users using V-TIMDER.

FIG. 25. Mixtures' fraction distributions. The total number of classified crops for each distribution is given in the legend.

FIG. 26. MiR mixture distributions visualization on the concentrations 2-simplex by a ternary plot of miR concentration distributions for the two experimental mixtures 1:1:1 (0.33:0.33:0.33) and 2:5:3 (0.2:0.5:0.3). Color represents the binned counts of simulated concentration values according to the multinomial and Gaussian error estimation described in the methods. The white contour lines represent Gaussians fitted to each of the distributions. The plot was generated using “Ternary Plots” from Matlab's file exchange functions.

FIGS. 27A-C. Ten spectral PSF combinations and classification demonstrated by 100 nm silica beads labeled with four fluorophores. A) Spectral FOV of multi-color beads as registered by CoCoS with RPA=177° B) The same FOV sequentially excited by four different lasers (405 nm, 488 nm, 561 nm, and 638 nm), registered separately, false colored and overlayed to generate a four-color image. C) Example PSFs cropped and placed side-by-side showcasing the ten different PSFs according to their fluorophore combinations.

FIG. 28. Crop augmentation by addition of weak Gaussian noise according to Table 10 parameters. Left column, three examples of the denoised miR crops. Right column, the same crops as the left column but with the addition of Gaussian noise. For each crop, six realizations of Gaussian augmented crops were made (see methods).

FIGS. 29A-B. Amplification-free detection of miR-15b-5p and miR-155 in small RNA extracted from 500 μL plasma. A) An example raw image of FOV containing the two miR targets. B) Zoom-in of the squared region in A, highlighting the two different PSFs, their corresponding miR targets identities and their fluorophore-pairs (in parentheses).

FIG. 30. Empirical PSF estimation crops. Empirical reference crops calculated from pixel-wise median of about 10\hat{ }5 noisy crops. These reference images were input to V-TIMDER (see FIG. 20) to facilitate the PSF visual classification process.

FIG. 31. A flowchart diagram of a classifier's pipeline.

FIG. 32. The first 20 PCA components generated by the unsupervised PCA analysis. For visual clarity, the components were symmetrically mirrored to generate 24×10 pixels crops (instead of the actual 24×5 pixels generated by the PCA after the symmetrization preprocessing in the classifier's pipeline, see methods). The three spectral PSFs (5,7- and 11-pixel distances) are clearly represented in the first 6 components, whereas the rest are probably attributed to noise classification. The diverging colormap used to present the PCA components was generated using the ‘lbmap’ function downloaded from Matlab file exchange 3.

FIG. 33. Confusion matrix results for the validation set.

FIG. 34. Confusion matrix results for the visually labeled 2:5:3 mixture dataset.

FIGS. 35A-C are schematic illustrations of light dispersion along one and two dimensions according to some embodiments of the present invention.

### DESCRIPTION OF SPECIFIC EMBODIMENTS OF THE INVENTION

The present invention, in some embodiments thereof, relates to methods of imaging biomolecules.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present embodiments comprise an optical detection scheme that allows simultaneous classification of a plurality of multiplexed biomolecules in one sample. The method relies upon fluorescently labeling the biomolecules, each with a unique fluorophore combination such that the combination of fluorophores per molecule is unique amongst all biomolecules which are labeled in the sample. The spectral signature of each of the combinations allows to create a “spectral barcode” that discloses the identity of the biomolecule.

The present embodiments are advantageous from the standpoint of time efficiency and cost-effectiveness.

Whilst reducing the present invention to practice, the present inventors showed that the method could be used to distinguish between synthetic mixtures of target microRNAs (miRs), and to diagnose CLL patients compared to healthy individuals based on the ratio of two target miRs.

Any biomolecule or molecular species may be detected as long as it is capable of being labelled with a fluorophore. Examples of molecular species that can be detected include polynucleotides (e.g. DNA molecules, RNA molecules, miRNA molecules), proteins (e.g. antibodies), peptides, carbohydrates, lipids.

In one embodiment, the method is used to distinguish between at least 2, 3, 4, 5, 6, 7, 8, 9 or 10 DNA species, each being of a different nucleic acid sequence. Each species is labelled with a unique pair of fluorophores to create an identification tag.

In one embodiment, the method is used to distinguish between at least 2, 3, 4, 5, 6, 7, 8, 9 or 10 RNA species, each being of a different nucleic acid sequence. Each species is labelled with a unique pair of fluorophores to create an identification tag.

In one embodiment, the method is used to distinguish between at least 2, 3, 4, 5, 6, 7, 8, 9 or 10 miRNA species, each being of a different s nucleic acid sequence. Each species is labelled with a unique pair of fluorophores to create an identification tag.

In one embodiment, the method is used to distinguish between at least 2, 3, 4, 5, 6, 7, 8, 9 or 10 proteins (e.g. antibodies), each being of a different amino acid sequence. Each species is labelled with a unique pair of fluorophores to create an identification tag.

The sample of the present invention may comprise a protein sample, a DNA sample, an RNA sample, a miRNA sample. In one embodiment, the sample is derived from a subject who is suspected of having a disease.

Synthetic fluorophores which may be used in the present invention may include, but are not limited to generic or proprietary fluorophores listed in Table A below:

TABLE A
Generic or proprietary exemplary fluorophores suitable for
use in the present invention

| | |
|---|---|
| Type 1 | Fluorescein and derivatives thereof, Rhodamine and |
| | derivatives thereof, Alexa Fluor ® dyes, DyLight Fluor ® |
| | dyes, Cyanine Cy ™ dyes, ATTO ® dyes, Abberior |
| | STAR ® dyes, Dyomics ® dyes, DNA fluorescent |
| | stains (for example, DAPI or 4′,6-diamidino-2- |
| | phenylindole), membrane fluorescent stains (for |
| | example, Dil or DilC₁₈(3), DiO or DiOC₁₈(3), DiD |
| | and DiR, which constitute a family of lipophilic |
| | fluorescent stains for labelling membranes and |
| | other hydrophobic structures). |
| Type 2 | A subset of Type 1 fluorophores, for example, Alexa |
| | Fluor ® 488, Alexa Fluor ® 555, Alexa Fluor ® 568, |
| | Alexa Fluor ® 647, Alexa Fluor ® 750, Alexa Fluor ® |
| | 790, ATTO ® 488, ATTO ® 520, ATTO ® 565, ATTO ® |
| | 647, ATTO ® 647N, ATTO ® 655, ATTO ® 680, |
| | ATTO ® 740, Cy2, Cy3, Cy3B, Cy3.5, Cy5, Cy5.5, Cy7, |
| | DyLight Fluor ® 750, Fluorescein Isothiocyanate (FITC), |
| | Dyomics ® 654 and IRDye ® 800CW. These dyes may |
| | change their fluorescent properties upon changes in the |
| | polarity of their environment. |
| Type 3 | Fluorescent proteins may include, but are not limited to, |
| | CFP, CyPET, GFP, YFP, YPET, RFP, and their mutants. |
| Type 4 | Photoactivatable or photoswitchable fluorescent proteins |
| | include, but are not limited to, PAGFP, Dronpa (and |
| | mutants such as Dronpa2, Dronpa3, Padron), rsFastLime, |
| | PAmCherry (and mutants PAmCherry1, PAmCherry2, |
| | or PAmCherry3, reCherry, rsCerryRev), PS-CFP1, |
| | PS-CFP2, Dendra1, Dendra2, Kaeda, KikGR, mKikGR, |
| | EosFP, mEos2, and KFP1. |
| Type 5 | Quantum dots, quantum rods. |
| Type 6 | Quenchers, for example the DYQ series by Dyomics ®, |
| | Black Hole Quencher Dyes by BioSearch Technologies ®, |
| | and the QSY series by ThermoFisher Scientific ®. |
| Type 7 | Caged fluorophores that can include, but not limited to, |
| | fluorophores that become fluorescent upon illumination |
| | with UV light. |
| Type 8 | Bioluminescent fluorophores may include, but are not |
| | limited to, Luciferase derived chimeras. |
| Type 9 | Chemiluminescent fluorophores. |
| Type 10 | Phosphorescent fluorophores may include, but are not |
| | limited to, lanthanides with or without sensitizers. |

It is expected that during the life of a patent maturing from this application many relevant fluorophores will be developed and the scope of the term fluorophores is intended to include all such new technologies a priori.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Methods for immobilization of oligonucleotides or proteins to solid-state substrates are well established. Oligonucleotides, including address probes and detection probes, and proteins (e.g. antibodies) can be coupled to substrates using established coupling methods.

Referring to the drawings, FIG. 14 is a schematic illustration of a system 10 which can be employed for executing a method that identifies a plurality of molecular species 12 in a sample 18, according to some embodiments of the present invention. The molecular species 12 shown in FIG. 14 are miRs (denoted miR-1, ad miR-2), but the present embodiments contemplate any molecular species capable of being labelled with a fluorophore, such as, but not limited to, polynucleotides (e.g. DNA molecules, RNA molecules, miRNA molecules), proteins (e.g. antibodies), peptides, carbohydrates, and lipids. In some embodiments of the present invention molecular species 12 are selected from the group consisting of a polypeptide, a polynucleotide, a lipid and a carbohydrate.

In some embodiments of the present invention molecular species 12 comprise a polynucleotide. In some embodiments of the present invention the polynucleotide is an RNA. In some embodiments of the present invention the RNA is a miRNA.

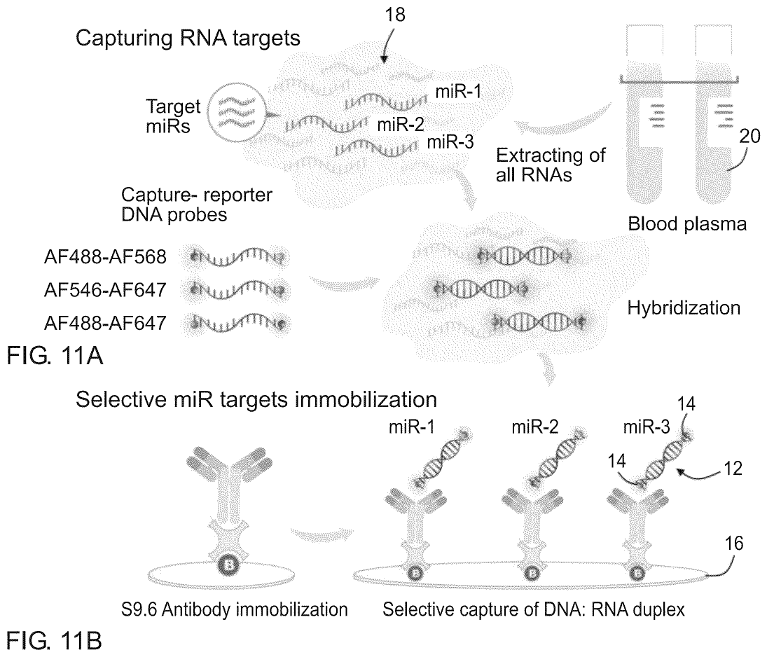

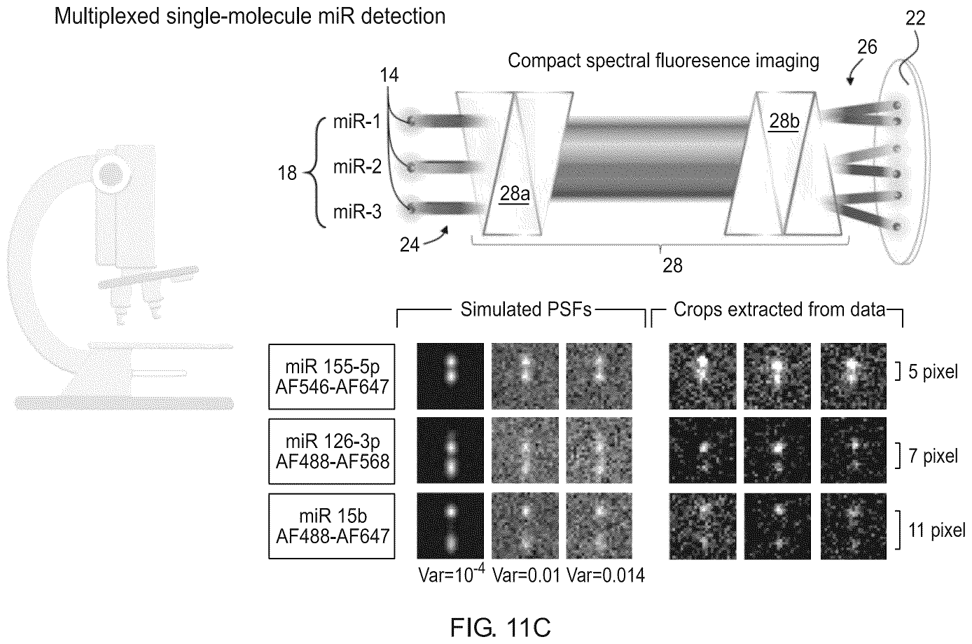

Sample 18 can be prepared in advance by extracting the molecular species 12 of interest from a source sample 20 (not shown in FIG. 14, see FIG. 11A), such as, but not limited to, a biological liquid, e.g., a blood sample comprising whole blood, serum, plasma, leukocytes or blood cells. Other types of source samples from which sample 18 can be prepared, including, without limitation, a urine sample, a saliva sample, a vaginal secretion sample, a feces sample, an interstitial fluid sample, a bacterial cell suspension, and a protein medium. In some embodiments of the present invention the sample is a cellular sample. In some embodiments of the present invention the sample is blood plasma. The molecular species 12 can be extracted from the source sample 20 by any technique known in the art, such as, but not limited to, using a commercially available kit. For example, small RNAs can be purified from blood plasma using a kit marketed as miRNeasy by QIAGEN GmbH, Hilden, Germany.

For each of at least a portion (two or more, more preferably three or more, more preferably four or more, more preferably five or more) of the molecular species 12 in sample 18, the respective molecular species 12 is labeled at a plurality of locations on the molecule with a respective plurality (e.g., two, three, four, five or more) of fluorophores 14. One or more of the molecular species 12 is preferably elongated so that the fluorophores form a sequence along the longitudinal direction of the labeled molecule.

Fluorophores 14 can be fluorophores that emit light by fluorescence emission or fluorophores that emit light by phosphoresce emission. Representative examples of fluorophores suitable for the present embodiments are provided in Table 1, above, and the Examples section that follows.

Two or more of the fluorophores 14 that are used to label a particular molecule optionally and preferably have different emission wavelengths. The combination of fluorophores and/or the spatial distance between the fluorophores on each labelled molecular species is unique for each of the labeled molecular species in the sample. This provides a plurality of uniquely labeled molecular species. In some embodiments of the present invention the combination of fluorophores 14 for a particular molecular species is unique among all other fluorophore-labeled molecular species in sample 18. In these embodiments, the distance between the locations of fluorophores 14 on molecular species 12 can be identical for two or more of the molecular species 12. Alternatively, two or more molecular species can be labeled with the same combination of fluorophores 14 but with a different spatial distance between the fluorophores on the respective molecular species.

Embodiments in which the combination of fluorophores 14 for a particular molecular species is unique among all other fluorophore-labeled molecular species in sample 18 are particularly useful when the spatial separation between the locations of the fluorophores along the molecule is small, e.g., at most 100 nm, or at most 50 nm, or at most 25 nm, or at most 10 nm. Embodiments in which the combination of fluorophores 14 for a particular molecular species need not be unique, but the spatial distance between the fluorophores on the molecules is unique among all other fluorophore-labeled molecular species in sample 18 are particularly useful when the spatial separation between the locations of the fluorophores along the molecule is sufficiently large e.g., 250 nm or more.

When there are more than two fluorophores that label a single molecule, the sequence of fluorophores may include repeats, or may be non-repetitive. Use of more than two fluorophores is particularly useful when the when the molecule is sufficiently large e.g., 200 nm or 250 nm in length or more.

The labeling can be by any labeling technique known in the art, including, without limitation, direct chemical conjugation, antibody labeling, genetic fusion, nanostructure conjugation, and biotin-streptavidin binding. In some embodiments of the present invention the molecular species 12 are labelled by hybridizing a probe that is labeled with two or more fluorophores to a polynucleotide. In some embodiments of the present invention the probe is a DNA probe. For example, when the molecular species 12 are miRs, total RNA can be extracted from a biological sample and selected miR targets can be specifically hybridized with DNA capture-reporter probes, each having a unique fluorophore-pair combination, providing hybridized complexes.

In some embodiments of the present invention the uniquely labeled molecular species are immobilized on a solid surface 16. Preferably, this is done after the labeling, but the present embodiments also contemplated cases in which the molecular species are immobilized on solid surface 16 and are thereafter labelled. While FIG. 14 illustrate use of more than one solid surface, according to a preferred embodiment of the invention a plurality of uniquely labeled molecular species are immobilized to a single solid surface, e.g., a microscope slide or the like. This embodiment is illustrated in FIG. 11B. In some embodiments of the present invention the molecular species are immobilized in a manner that they are oriented generally along each other, or along a predetermined direction (e.g., the y direction in a Cartesian coordinate system). This can be ensured by generating a flow of the molecular species along a particular direction which forces the molecules to orient along the direction of the flow, and executing the immobilization operation during the flow. Also contemplated are embodiments in which the molecules are oriented by means of surface tension or electric field. In some embodiments of the present invention the uniquely labeled molecular species are not immobilized. For example, they can be allowed to flow within fluidic channels, such as, but not limited to, nanochannels. Alternatively, they can be allowed to flow within fluidic channels and immobilized thereafter.

A representative example of an immobilization technique suitable for the present embodiments include, without limitation, selectively capturing the species (or complexes) on a surface by means of an antibody, more preferably a monoclonal antibody. Other example immobilization techniques include, without limitation, adsorption, covalent immobilization, physical entrapment, and microarray printing.

In some embodiments of the present invention, the immobilization comprises selectively immobilizing only the uniquely labeled molecular species on the solid surface 16, whilst not immobilizing non-labeled molecular species on solid surface 16. This can be achieved by preparing the surface such that it includes moieties (e.g., antibodies) that are attached to the surface and that specifically bind to the molecular species of interest.

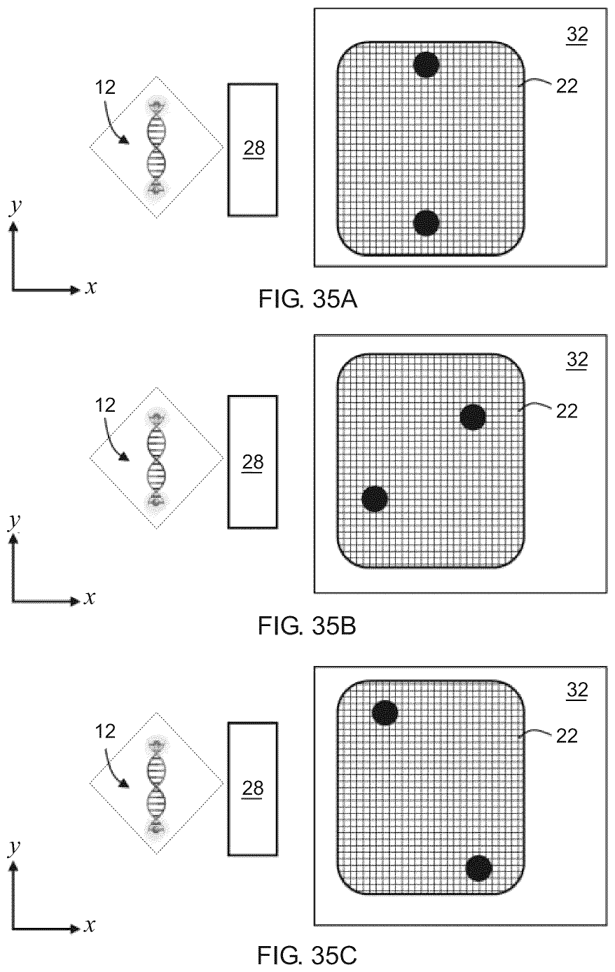

Once the molecular species 12 are labeled, and optionally and preferably immobilized, light 24 emitted from fluorophores 14 is dispersed 26 onto an imager 22. Imager 22 is illustrated in FIGS. 35A-C, and its generated image can be displayed on a computer screen 30, as illustrated in FIG. 14. The imager is preferably pixelated light sensor (e.g., a MOS imager, a CMOS imager, a sCMOS imager, a CCD, an EMCCD, an InGaAs imager, or combination thereof).

As used herein, “dispersion” refers to a phenomenon in which different spectral components of light travel at different speeds through a medium, causing a separation of the spectral components in a manner that spectral components having different wavelengths propagate along different directions.

Preferably, light from two or more labeled molecular species, more preferably all labeled molecular species is dispersed onto the imager to form a single image of fluorophore emissions. The advantage of these embodiments is that they allow simultaneous identification of two or more molecular species, unlike conventional systems which are based on emission filters and in which the imaging is sequential wherein each emission wavelength is imaged separately.

The dispersion of the light 24 is by an optical dispersion system 28, which can include any optical component that is known to cause dispersion, including, without limitation, a prism, a double prism, a diffraction grating, a Fresnel prism, a grism, and a photonic crystal. In some embodiments of the present invention optical dispersion system 28 is constituted to disperse light 24 in a manner that the different spectral components of light 24 exit system 28 separated from each other and propagating parallel to each other. Alternatively, optical dispersion system 28 is constituted to disperse light 24 in a manner that the different spectral components of light 24 exit system 28 propagating along different direction. For example, a double prism can be arranged in a manner that the different spectral components of light 24 exit system 28 separated from each other and propagating parallel to each other. FIG. 11C illustrates a configuration in which system 28 employs two prism assemblies 28a and 28b, wherein the first prism assembly 28a receives light 24 and output its component propagating parallel to each other, and the second prism assembly 28b receives the parallel propagating components and outputs each component along a different direction.

In some embodiments of the present invention the dispersing comprises non-linear dispersing, and in some embodiments of the present invention the dispersing comprises linear dispersing. Non-linear dispersion is a type of dispersion where the relationship between the wavelength and the propagation velocity of light is not linear, and linear dispersion is a type of dispersion where the relationship between the wavelength and the propagation velocity of light is linear. Typically, whether the dispersion is linear or non-linear depends on the material through which light propagate in system 28. Linear dispersion can be achieved by allowing the light to propagate through glass and various polymeric materials, e.g., PMMA. Non-linear dispersion can be achieved by allowing the light to propagate through a non-linear optical crystal.

The advantage of the dispersion of the light 24 is that it increases the distance between the images of the spectral components on the imager, thereby allowing to spatially resolve the images of the different fluorophores 14 of each molecular species 12, which would otherwise overlap or be too close to be distinguished. For example, when the molecular species 12 include miRs, and the labeling is by means of a DNA probe having fluorophores, the largest distance between the locations of two fluorophores on the DNA probe is the length of the DNA probe. A typical DNA probe has a length of a few tens of base pairs, which is equivalent to a several nanometers. Such a distance is smaller than the diffraction limit of commercially available cameras and would therefore be captured at the same addressable location on the imager. The dispersion of light 24 according to the present embodiments ensures that the fluorophores of a given molecule are captured at different addressable locations on the imager, making them distinguishable.

The presence embodiments contemplate more than one way to disperse the light, as will now be explained with reference to FIGS. 35A-C.

In some embodiments of the present invention the dispersing is one-dimensional wherein the direction of the one-dimensional dispersing is generally parallel (e.g., with a tolerance of about 10°) to a direction along which fluorophores 14 are arranged over molecular species. These embodiments are illustrated in FIG. 35A, where the molecule 12 is oriented along the y direction, and the one-dimensional dispersing is also along the y direction, providing a larger distance along the y direction between the images of fluorophores 14 on imager 22 than distance along the y direction between fluorophores 14 themselves.

In some embodiments of the present invention the dispersing is one-dimensional wherein the direction of the one-dimensional dispersing is non-parallel (e.g., more than about 20°) to a direction along which fluorophores 14 are arranged over molecular species. These embodiments are illustrated in FIG. 35B, where the molecule 12 is oriented along the y direction, and the one-dimensional dispersing is also along the x direction (orthogonal to the molecule), providing a separation along the x direction between the images of fluorophores 14 on imager 22, where no such separation along the x direction exists between fluorophores 14 themselves.

In some embodiments of the present invention the dispersing is two-dimensional. These embodiments are illustrated in FIG. 35C, where the molecule 12 is oriented along the y direction, and the dispersing is also along both the y direction (parallel to the molecule) and the x direction (orthogonal to the molecule), providing a separation between the images of fluorophores 14 on imager 22 along both the x and y directions.

The imager 22 on to which the dispersed light is directed is a component of an imaging system 32. The largest among the spatial separations between the fluorophores 14 themselves (along the molecules 12) is preferably less than diffraction limit of system 32, and the smallest among the distances between the images of fluorophore 14 (on imager 22) is preferably larger than the diffraction limit of system 32.

The imaging system 32 thus generates an image of the fluorophore emissions from the uniquely labeled molecular species 12. In some embodiments of the present invention the image is a spectral image. The image is spectral in the sense that it provides information regarding the position of the labeled molecular species 12 as well as regarding the emission wavelengths. In these embodiments, for any pair of emission wavelengths, a distance between fluorophore emissions corresponding to that pair is unique among all other pairs. Since the distance is unique and the emissions wavelengths are known, the image provides information regarding the emission wavelengths, and is therefore a spectral image.

The method can then correlate distances between fluorophore emissions in the image to the molecular species 12 in sample 18, wherein the correlation identifies the molecular species 12. The method preferably identify the species 12 at a single molecule level, namely identify the type of individual molecules in sample 18. In some embodiments of the present invention the intensity distribution of the imaged fluorophore emissions is analyzed and is also correlated to the molecular species. For example, the method can calculate a multi-spot Point Spread Function (PSF) for each combination of fluorophore emissions, and then measure distances between the individual spots of the calculated multi-spot PSF thereby also determining the distances between fluorophore emissions. The number of spots in the multi-spot PSF is based on the number of fluorophores used to label the molecular species. Generally, for a combination of n fluorophores labeling a particular molecular species the method can calculate an n-spot PSF. For example, when a particular molecular species is labeled with two fluorophores, the method calculates a dual-spot PSF, and when a particular molecular species is labeled with three fluorophores, the method calculates a triple-spot PSF, and so on.

In some embodiments of the present invention the labelled molecular species are quantified from the produced image. This is optionally and preferably done by calculating a count distribution for each identified molecular species, wherein the count distribution of a particular labelled molecular species quantifies that molecular species.

The method of the present embodiments can also be used for diagnosing a disease of a subject. In these embodiments, the presence and/or an amount of one or more molecular species in the image is indicative of the disease. Representative examples of types of diseases that can be diagnosed based on the presence and/or an amount of molecular species in the image, include, without limitation, cancers, cardiovascular diseases, heart failure, neurological disorders, metabolic disorders, infectious diseases (particularly, but not exclusively viral infections), autoimmune diseases, hyperlipidemia, metabolic syndromes, and endocrine disorders.

The correlation and optional intensity analysis and/or quantification are preferably executed automatically by an image processor having a circuit configured to receive the image, and process it to analyze the intensity distribution, measure the distances, correlate between the distances and the species, and optionally and preferably also quantify the species.

In the simplest case, the correlation is by means of a lookup table having a plurality of entries each including a distance over the image and a type of molecular species. Such a lookup table provides a mapping between intra-pair distances and labeled molecular species. The methods process the image to measure distances between fluorophore emissions, search the lookup table for entries that match the measure distances, and identifies the type of molecular species using the type of molecular species in those entries. Use of a lookup table is particularly useful when the dispersion is one-dimensional, but may also be used when the dispersion is two-dimensional.

Alternatively, the correlation is by means of a machine learning procedure trained to receive an image and classify its content in terms of the molecular species that are imaged in the image.

As used herein the term “machine learning” refers to a procedure embodied as a computer program configured to induce patterns, regularities, or rules from previously collected data to develop an appropriate response to future data, or describe the data in some meaningful way.

Representative examples of machine learning procedures suitable for the present embodiments, include, without limitation, clustering, association rule algorithms, feature evaluation algorithms, subset selection algorithms, support vector machines (SVMs), classification rules, cost-sensitive classifiers, vote algorithms, stacking algorithms, Bayesian networks, decision trees, neural networks (e.g., fully-connected neural network, convolutional neural network), instance-based algorithms, linear modeling algorithms, k-nearest neighbors (KNN) analysis, ensemble learning algorithms, probabilistic models, graphical models, logistic regression methods (including multinomial logistic regression methods), gradient ascent methods, singular value decomposition methods and principle component analysis (PCA).

A machine learning procedure can be trained according to some embodiments of the present invention by feeding a machine learning training program with training data including image patches or crops and a respective classification for each image patch or crop. The classification can include identification of the molecular species that are imaged in the respective patch or crop. Once the training data are fed, the machine learning training program generates a trained machine learning procedure which can then be used without the need to re-train it.

The present embodiments contemplate any type of machine learning procedure, including a combination of two or more such machine learning procedures. For example, an image can be classified using a combination of a PCA and an SVM, wherein the PCA is applied to the image and the SVM is applied to a processed image obtained by projecting the image onto a plurality of principle components obtained by the PCA.

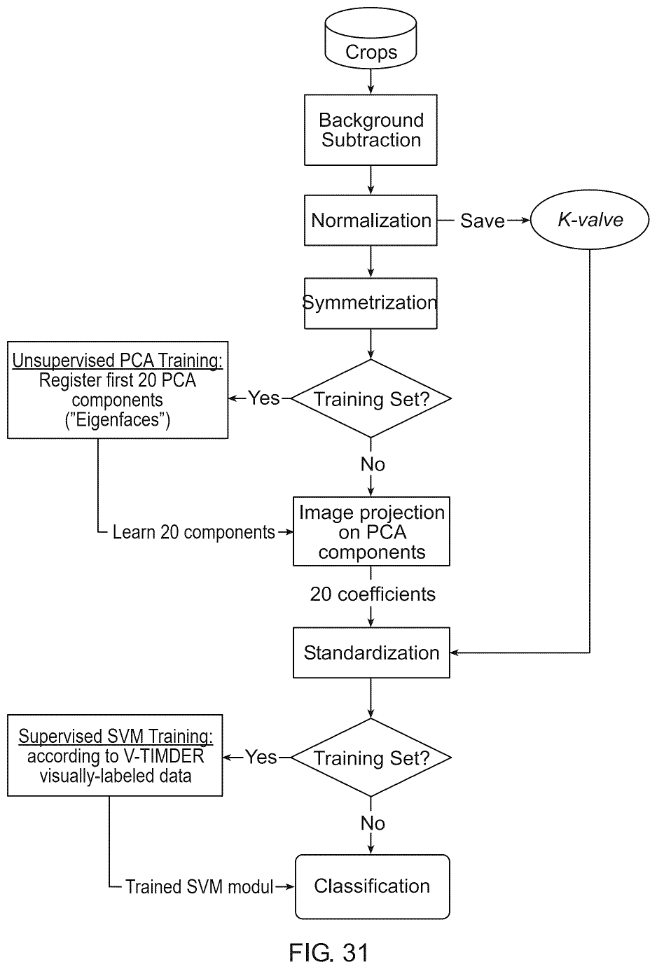

Use of a machine learning procedure is useful both when the dispersion is one-dimensional and when the dispersion is two-dimensional. Representative examples of machine learning procedures that can be used according to some embodiments of the present invention is provided in the Examples section that follows. Example flowchart describing use of PCA followed by SVM is provided in FIG. 31 of Example 3, below.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “diagnosing” refers to determining presence or absence of the disease, classifying the disease, determining a severity of the disease, monitoring disease progression, forecasting an outcome of a pathology and/or prospects of recovery and/or screening of a subject for the disease.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. References are provided throughout this document. The procedures therein are believed to be well known in the art and are provided for the convenience of the reader. All the information contained therein is incorporated herein by reference.

### Example 1

### Materials and Methods:

Slide preparation: The miRs were captured on borosilicate glass coverslips (D 263 Schott glass, 75.5×25.5 mm², ibidi GmbH, Germany), passivated with poly-ethylene glycol (PEG). In brief, after cleaning, each coverslip was hydroxyl terminated with freshly prepared KOH. Afterwards they were pegylated with a mixture of Methoxy PEG Silane (Laysan Bio Inc. AL, USA) and mPEG-Silane-Biotin, MW5000 (Laysan Bio Inc. AL, USA) in a 1:100 stoichiometry in dehydrated HPLC grade Ethanol. A six channel ibidi sticky-slide (μ-Slide VI 0.4, ibidi GmbH, Germany) was mounted on top of pegylated coverslip. Each channel was hydrated with 100 μl of DNase I and RNase-free deionized water for 15 minutes, and then equilibrated with 100 μl PBS for 30 min. Following this, each channel was activated with 100 ul of monoclonal Anti-DNA-RNA hybrid S9.6 antibody (S9.6, Mouse IgG2a kappa Isotype, conjugated with Streptavidin, Ab01137-2.0, Absolute Antibody, Oxford, UK) to capture our targeted biomarkers. Thus prepared channels were able to capture targeted miRs hybridized with labelled DNA probes as DNA:RNA duplex.

Probe sequences:
Capture probe for hsa-miR-15b-5p
(ATTO488-ATTO647N):
(SEQ ID NO: 1)
/5ATTO488K/TT AGT TGT AAA CCA TGA TGT GCT
GCT AAT GTA/3ATTO647NN/
Capture probe for hsa-miR-155-5p
(AF546-ATTO647N):
(SEQ ID NO: 2)
/5Alex546N/TT AGT AAC CCC TAT CAC GAT TAG
CAT TAA ATG TA/3ATTO647NK/
Capture probe for hsa-miR-126-3p
(ATTO588-ATTO565N):
(SEQ ID NO: 3)
/5ATTO488N/AC TTA GTC GCA TTA TTA CTC ACG
GTA CGA ATG TAT C/3ATTO565N/

Synthetic miR experiments: In all control experiments with synthetic microRNAs (miRs) and their complimentary single stranded DNA capture probes (ssDNA), 100 ul of duplex at 50 pM concertation is used in each channel which was preincubated with S9.6 antibody.

CLL diagnosis experiment: All small RNAs (including microRNAs/miRs) were purified from 500 μl plasma using a miRNeasy Serum/Plasma Advanced Kit (QIAGEN GmbH, Hilden, Germany) in 15 μl of Rnase free water, and stored in −20° C. To this purified miRs, 15 μl of PBS was added, following 0.1 fmol (1 μl of 5 pM) of each of the capture probes, and later hybridized at room temperature for 3 hours. For these experiments we used two capture probes targeting two human miRs; viz. hsa-miR-15b-5p dual labelled with alexa 488 and Atto 647 dye, and hsa-miR-155-5p dual labelled with alexa 546 and Atto 647 dye. After hybridization, total 31 μl of miR mixture is applied on the immobilized S9.6 antibody in one channel, incubated for 45 min, and washed 3× with PBS before imaging.

### Optical Setup

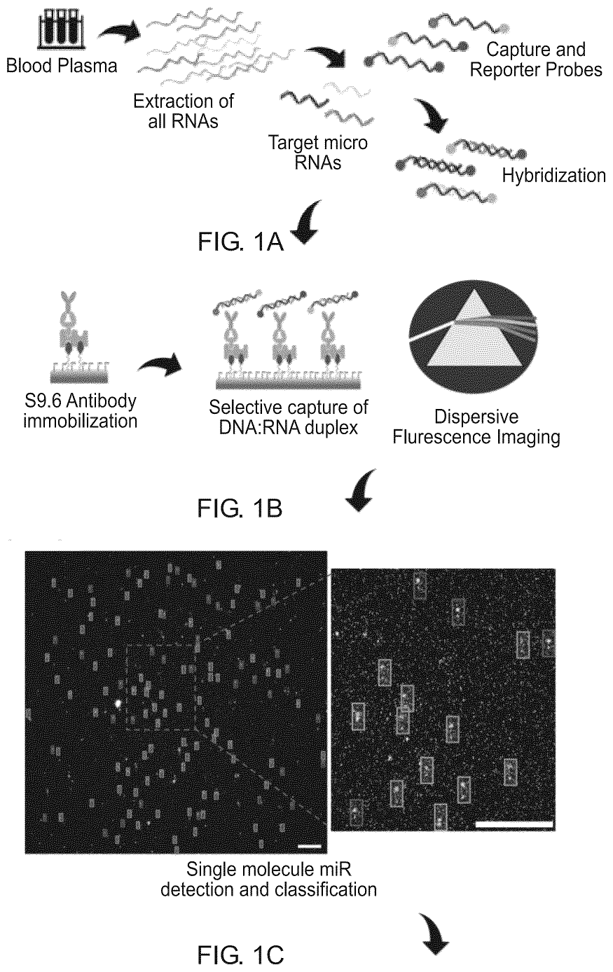

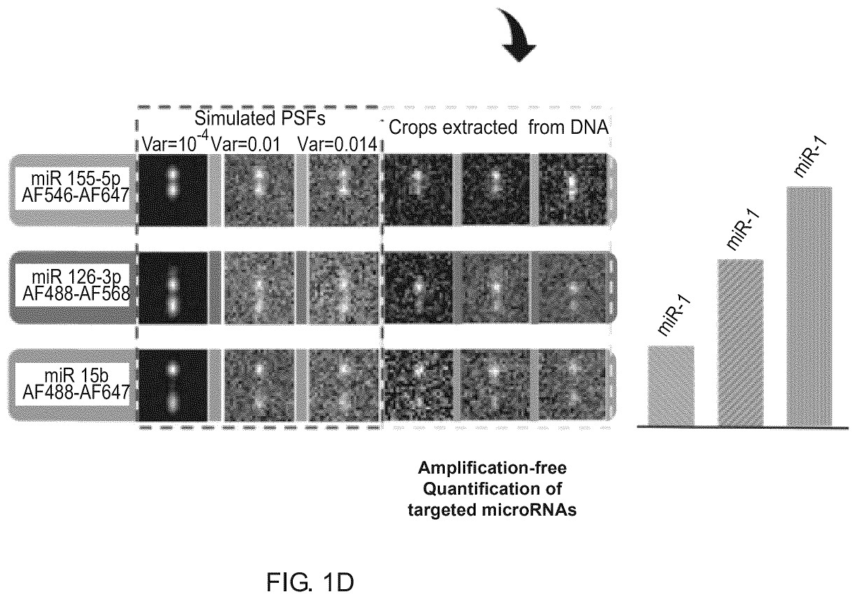

Excitation: For excitation we used three lasers (Cobolt AB, Sweden) with wavelengths 488 nm (MLD 488, 200 mW max power), 561 nm (Jive 561, 500 mW max power), 638 nm (MLD 638, 140 mW max power). All lasers were mounted on an in-house designed heatsink which coarse aligned their beam heights. Each laser beam was passed through a clean-up filter (LL01-488-12.5, LL01-561-12.5, LL01-638-12.5, Semrock, USA) and expanded to 12.5-20× its original diameter (3×LB1157-A, 3×LB1437-A, Thorlabs, USA). A motorized shutter (SH05, Thorlabs, USA) was used for modulating on/off the solid-state 561 nm laser, while the diode lasers were modulated directly on the laser head. The beams were then combined into a single beam using long-pass filters (Di03-R488-t1-25.4D, Di03-R561-t1-25.4D, Semrock, USA). To homogenize the excitation profile of the sample, the combined beam was passed through an identical setup to the one described in the work of Douglass et al.²⁷. In short, the combined beam was injected into a compressing telescope (AC254-150-A-ML, AC254-050-A-ML, Thorlabs, USA) with a rotating diffuser (24-00066, Süss MicroOptics SA, Switzerland) placed about 5 mm before the shared focal points of the telescope lenses (see FIG. 1B). A series of 6 silver mirrors (PF10-03-POI, Thorlabs, USA) was then used to align the beam into a modified microscope frame (IX81, Olympus, Japan), through two identical microlens arrays (2×MLA, 18-00201, Süss MicroOptics SA, Switzerland) separated by a distance equal to the microlenses focal length and placed inside the microscope frame. The homogenized beam was reflected onto the objective lens (UPlanXApo 60×NA1.42, Olympus, Japan) by a four-band-multichroic mirror (Di03-R405/488/532/635, Semrock, USA). The sample was placed on top of motorized XYZ stage (MS-2000, ASI, USA) with an 890 nm light-emitting diode (LED) based autofocus system (CRISP, ASI, USA), which enabled scanning through multiple fields of view.

Emission: The emitted fluorescence light was gathered by the same objective and transmitted through the multichroic mirror onto a standard Olympus tube lens to create an intermediate image at the exit of the microscope frame. This image was passed through a multi-band emission filter (FF01-440/521/607/694/809-25, Semrock, USA) and was then directed into a magnifying telescope (Apo-Rodagon-N 105 mm, Qioptiq GmbH, Germany and Olympus' wide field tube lens with 180 mm focal length, #36-401, Edmund Optics, USA), with two commercial direct vision prisms (117240, Equascience, France) placed within the infinity space between the lenses and mounted on two motorized rotators (8MR190-2-28, Altechna UAB, Lithuania) controlling the prisms' angles around the optical axis. The final image was acquired on a back illuminated sCMOS camera (Prime BSI, Teledyne Photometrics, USA).

Image acquisition was coordinated using the micro-manager software²⁸, controlling camera acquisition, laser excitation, XY stage location, and prism rotator angles. The camera and lasers excitation were synchronized using an in-house built TTL controller based on an Arduino® Uno board (Arduino AG, Italy)²⁹.

Image acquisition: The sample lanes were scanned laterally and imaged with a single acquisition per FOV, obtaining approximately about 2000 FOVs per sample. The acquisition parameters are described in Table 1, herein below:

TABLE 1

| | | Emission | Exposure | Laser |
|---|---|---|---|---|
| Excitation | RPA | Filter | time | Intensities |
| All lasers | 177.5 | FF01-440/521/607/ | 800 ms | 638 Laser- |
| simultaneously | | 694/809-25 | | 90 mW |
| | | | | 561 Laser- |
| | | | | 90 mW |
| | | | | 488 Laser- |
| | | | | 160 mW |

Image analysis: The detection and classification of the miRs according to their point spread functions (PSFs) was performed in two steps:

First, to remove the non-homogeneous background in each FOV, for each experiment we subtracted a pixel-wise median (calculated by FIJI's Z-project function) from all FOVs in the experiment.

Next, by manual curation of all two-spots PSFs we were able to classify them to their respective miR target.

PSFs simulations: All simulations were performed by a home-built Matlab code.

Table 2 lists the dyes used in the experiment.

TABLE 2

| Name | AF488 | ATTO520 | AF514 | 6-JOE | Cy3 | AF546 | ATTO550 | AF568 |
|---|---|---|---|---|---|---|---|---|
| Ex. max (nm) | 493 | 496 | 517 | 519 | 520 | 554 | 554 | 561 |
| Em. Max (nm) | 517 | 538 | 543 | 548 | 566 | 572 | 577 | 603 |
| Ex. Factor | 0.94 | 0.42 | 0.43 | 0.38 | 0.93 | 1.09 | 0.90 | 0.69 |
| Name | AF594 | ATTO490LS | AF647 | AF660 | AF680 | ATTO700 | AF700 | AF750 |
| Ex. max (nm) | 579 | 590 | 653 | 663 | 679 | 696 | 700 | 752 |
| Em. Max (nm) | 618 | 658 | 670 | 691 | 702 | 719 | 719 | 779 |
| Ex. Factor | 0.54 | 1.04 | 0.79 | 1.15 | 0.74 | 0.44 | 0.56 | 0.22 |

Each of the dye's spectrum was multiplied with the filter's spectrum to produce the actual spectrum visible on our camera.

The wavelength to pixels displacement calibration curve of our controlled spectral-resolution (CoCoS) setup (which was calculated previously²⁶ was adjusted according to the experimentally used RPA by multiplying the entire curve by sin((180-RPA)/2).

Chosen double-dye combinations were then simulated by converting each dye's spectrum into a diffraction limited dispersed image. This was done by assigning a gaussian with unity amplitude and 1.15 pixel standard deviation to each wavelength in the emission spectrum. Each gaussian was displaced according to the RPA-adjusted displacement curve and summed together with other gaussians. Finally, the total summed intensity of all gaussians was normalized to unity and multiplied by an excitation efficiency factor which was calculated by the excitation spectrum value (fractions only) at the excitation laser wavelength.

This process was repeated for the second dye and both images were summed to provide the dual-dye spectral image.

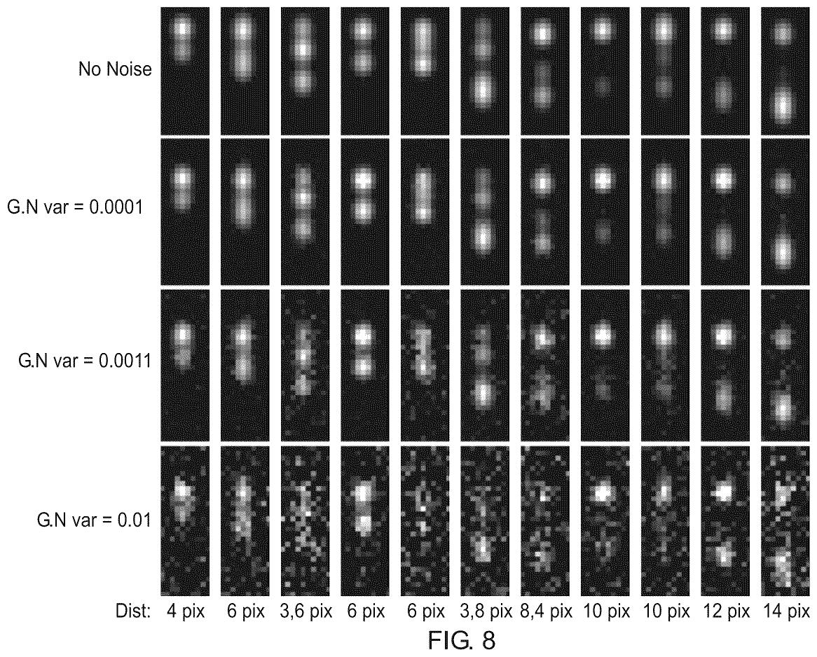

When needed noise model was added to the simulated dual-dye spectral PSF image using the imnoise function in Matlab. The noise model used in this work was a sum of a poisson distributed shot-noise and gaussian noise with a constant mean of 0.3 and changing variance (FIG. 8).

### Results

FIGS. 1A-B provide an overview of the method used for multiplexed single-molecule detection and profiling of a panel of disease-associated miRs.

The method is built upon three pillars: (1) multiplexed fluorescence labeling of up to 10 miR targets, (2) specific binding of these targets to the imaging surface, and (3) imaging and classification of all targets simultaneously at a single-molecule sensitivity. Combined together, these features allow for fast, sensitive, cost-effective, and robust profiling of small panels of miR targets.

The miR targets are labeled by a panel of up to 10 unique DNA reporter probes. The reporter probes are designed to have a 30 base pairs sequence, each with complementing sequence to a specific miR target. Each probe is labeled with a unique two dyes combination, positioned at the opposite ends of the construct to avoid non-radiative energy transfer between them (e.g., by Forster resonant energy transfer). The spectral signature of each of the dye-duo combinations allows creation of a “spectral barcode” that uncovers their target miR's identity.

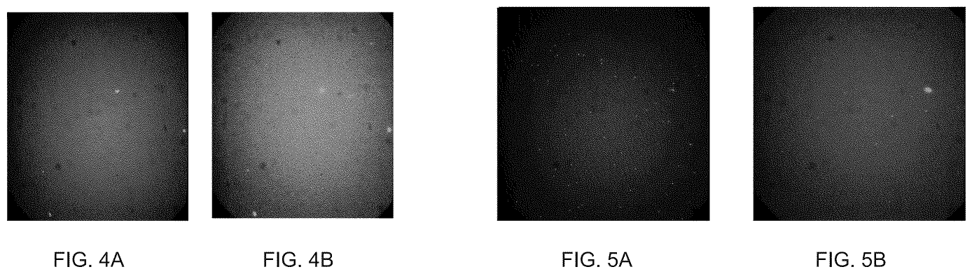

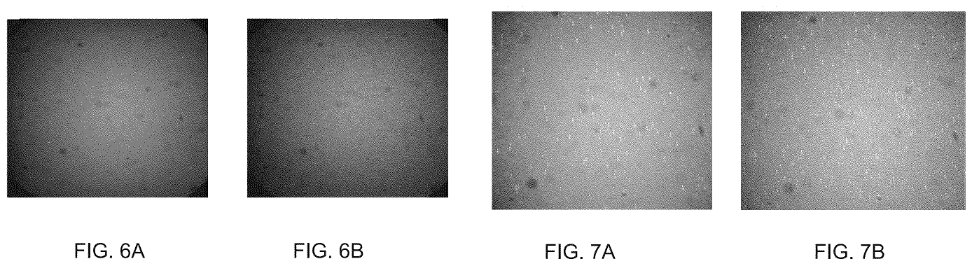

The specific binding of target miRs to the imaging surface is achieved by functionalizing microscope coverslips with a monoclonal Anti-DNA-RNA hybrid [S9.6] antibody substrate²⁵. The antibody selectively captures only DNA:RNA constructs which correspond to miR targets and reporter probes hybrids, allowing to image only the target miRs. After the miR-reporter constructs are captured on the surface, subsequent washes remove all excess unhybridized reporter probes and significantly reduce background noise. This capture process eliminates non-specific binding of reporter probes, as was thoroughly validated in control experiments (see FIGS. 4A-B-7A-B for control experiments showing the binding specificity), and allows for a sensitive readout of the relevant targets.

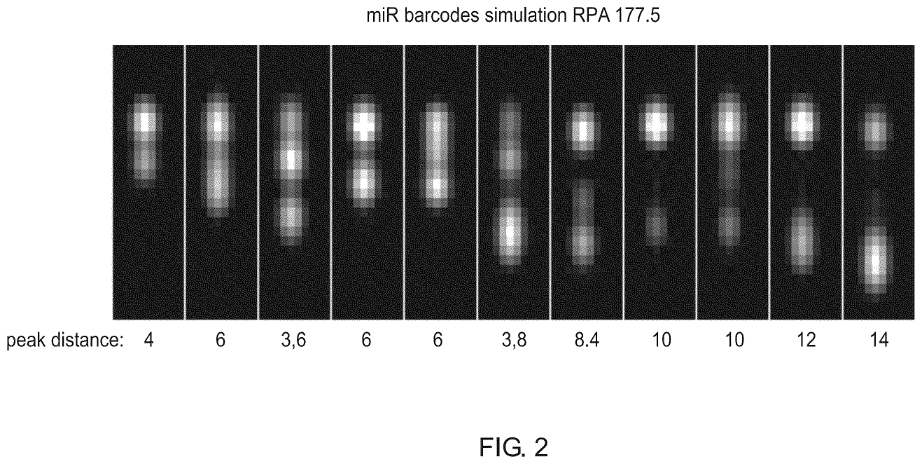

The captured miR:reporter hybrids with their unique dye-pair combinations are imaged and resolved by miRacle's spectral imaging module based on the previously introduced CoCoS system²⁶. With it all targets can be imaged simultaneously with a single frame acquisition per field of view (130×130 μm²) containing hundreds of miRs. Each miR target is detected as a unique 2-spot point-spread-function (PSF) corresponding to the spectral emission signature of its dye-pair (FIG. 1D and FIG. 2). The unique distance between the spots and their intensity distribution allows for a one-to-one classification of the spectral PSF and the target miR. CoCoS allows to easily control the spectral dispersion introduced to the image, therefor for each panel of reporter probes, we can optimally set the minimal spectral resolution that allows to resolve the reporters used in each experiment, increasing both signal to noise ratio (SNR) and the possible resolvable reporter density without PSFs overlaps. Overall, this optimized spectral imaging enables multiplexed registration of single molecule miRs.

The number of miR targets that can be multiplexed in a single sample is dictated by the ability to correctly classify the reporter probes' PSFs. The PSFs can be designed according to the required amount of miR targets, by adjustment of miRacle's spectral resolution and/or the probes' dye-duo spectra. To guarantee our ability to multiplex and classify different miR targets, we designed a simple PSF simulator in Matlab (FIGS. 1D and 2). The simulator gets as an input the dyes and filters' spectra together with the induced spectral dispersion by CoCoS. It then simulates the dye-duo PSFs with an option of adding Gaussian and Poisson noises which provide a realistic assessment of our classification capability (FIG. 1D and FIG. 8). This allows to choose the best dispersion and dye-pair combinations according to the experiment and the required number of miR targets.

To test the performance of our platform, we used synthetic microRNA samples containing three different miR targets miR-15b-5p, miR-155-5p and miR-126-3p) to quantify their expression levels. Specifically, we imaged each of the synthetic microRNA species separately to register the probes' PSF spectral signature and compared it to our simulated theoretical prediction (FIG. 1D). We then quantified the expression of each target in two mixture samples of known concentrations and quantified their relative abundance. This allowed us to assess the accuracy and sensitivity of our platform for microRNA quantification in a controlled setting.

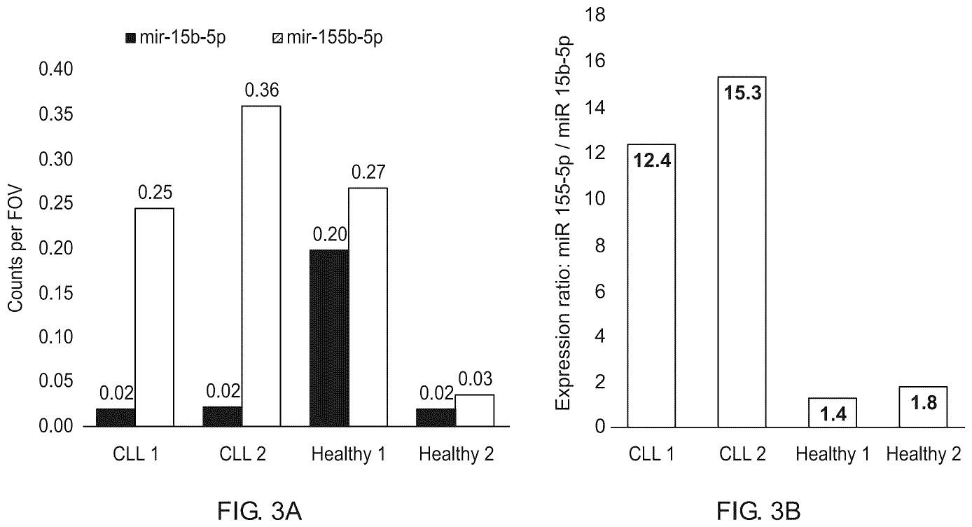

To show miRacle diagnostic capability in a clinical setting, we used the platform to diagnose chronic lymphocytic leukemia (CLL) in five plasma samples (2 CLL patients and 2 healthy controls). With a sufficiently sensitive detection, a subset of two miR targets (miR-155-5p and miR-15b-5p) can discriminate between CLL patients and healthy individuals¹². In CLL, miR-155-5p is highly upregulated while miR-15b-5p is downregulated, therefore, their expression ratio provides an accurate measure of a patient's state. To classify our samples, we assessed >1000 FOVs per sample, each comprising hundreds of single-molecule miRs, as summarized in Table 3, herein below. By using the ratio between the overall abundance of the two miR targets we were able to correctly classify the 4 samples to be either CLL patients or healthy controls (FIGS. 3A-B).

TABLE 3

| | | | | | | miR-15b- | miR-155- |
|---|---|---|---|---|---|---|---|
| | | | miR- | miR- | | 5p | 5p |
| Sample | Sample | number of | 15b-5p | 155-5p | miR Ratio | Counts | Counts |
| Name | number | FOVs counted | Counts | Counts | 155/15p | per FOV | per FOV |
| CLL 1 | 892224 | >500 | | | | | |
| CLL 2 | 892220 | 1064 | 21 | 261 | 12.43 | 0.02 | 0.25 |
| CLL 3 | 892219 | 1659 | 39 | 598 | 15.33 | 0.02 | 0.36 |
| Healthy | 892232 | 500 | 99 | 134 | 1.35 | 0.20 | 0.27 |
| 1 | | | | | | | |
| Healthy | 892125 | 1386 | 26 | 48 | 1.85 | 0.02 | 0.03 |
| 2 | | | | | | | |

### REFERENCES FOR EXAMPLE 1

1. Ha, M. & Kim, V. N. Regulation of microRNA biogenesis. Nat. Rev. Mol. Cell Biol. 15, 509-524 (2014).
2. Bartel, D. P. Metazoan MicroRNAs. Cell 173, 20-51 (2018).
3. Friedman, R. C., Farh, K. K. H., Burge, C. B. & Bartel, D. P. Most mammalian mRNAs are conserved targets of microRNAs. Genome Res. 19, 92-105 (2009).
4. Lujambio, A. & Lowe, S. W. The microcosmos of cancer. Nature 482, 347-355 (2012).
5. Goodall, G. J. & Wickramasinghe, V. O. RNA in cancer. Nat. Rev. Cancer 2020 211 21, 22-36 (2020).
6. Wu, Y. et al. Circulating microRNAs: Biomarkers of disease. Clin. Chim. Acta 516, 46-54 (2021).
7. Schwarzenbach, H., Hoon, D. S. B. & Pantel, K. Cell-free nucleic acids as biomarkers in cancer patients. Nat. Rev. Cancer 2011 116 11, 426-437 (2011).
8. Balatti, V., Pekarky, Y. & Croce, C. M. Role of microRNA in chronic lymphocytic leukemia onset and progression. J. Hematol. Oncol. 8, 1-6 (2015).
9. Drees, E. E. E. & Pegtel, D. M. Circulating miRNAs as Biomarkers in Aggressive B Cell Lymphomas. Trends in cancer 6, 910-923 (2020).
10. Elias, K. M. et al. Diagnostic potential for a serum miRNA neural network for detection of ovarian cancer. Elife 6, (2017).
11. Calin, G. A. et al. A MicroRNA Signature Associated with Prognosis and Progression in Chronic Lymphocytic Leukemia. N. Engl. J. Med. 353, 1793-1801 (2005).
12. Filip, A. A. et al. Expression of circulating miRNAs associated with lymphocyte differentiation and activation in CLL—another piece in the puzzle. Ann. Hematol. 96, 33-50 (2017).
13. Precazzini, F., Detassis, S., Imperatori, A. S., Denti, M. A. & Campomenosi, P. Measurements methods for the development of microRNA-based tests for cancer diagnosis. International Journal of Molecular Sciences 22, 1-27 (2021).
14. Kim, D. J. et al. Plasma components affect accuracy of circulating cancer-related microRNA quantitation. J. Mol. Diagnostics 14, 71-80 (2012).
15. Pritchard, C. C., Cheng, H. H. & Tewari, M. MicroRNA profiling: approaches and considerations. Nat. Rev. Genet. 2012 135 13, 358-369 (2012).
16. Fu, Y., Dominissini, D., Rechavi, G. & He, C. Gene expression regulation mediated through reversible m6A RNA methylation. Nat. Rev. Genet. 15, 293-306 (2014).
17. Deng, S. et al. RNA m6A regulates transcription via DNA demethylation and chromatin accessibility. Nat. Genet. 54, 1427-1437 (2022).
18. Taylor, S. C. et al. The Ultimate qPCR Experiment: Producing Publication Quality, Reproducible Data the First Time. Trends Biotechnol. 37, 761-774 (2019).
19. Hong, L. Z. et al. Systematic evaluation of multiple qPCR platforms, NanoString and miRNA-Seq for microRNA biomarker discovery in human biofluids. Sci. Rep. 11, 4435 (2021).
20. Mestdagh, P. et al. Evaluation of quantitative miRNA expression platforms in the microRNA quality control (miRQC) study. Nat. Methods 11, 809-15 (2014).
21. Prokopec, S. D. et al. Systematic evaluation of medium-throughput mRNA abundance platforms. RNA 19, 51-62 (2013).
22. Cheng, Y., Dong, L., Zhang, J., Zhao, Y. & Li, Z. Recent advances in microRNA detection. Analyst 143, 1758-1774 (2018).
23. Geiss, G. K. et al. Direct multiplexed measurement of gene expression with color-coded probe pairs. Nat. Biotechnol. 26, 317-325 (2008).
24. Narrandes, S. & Xu, W. Gene expression detection assay for cancer clinical use. J. Cancer 9, 2249-2265 (2018).
25. Boguslawski, S. J. et al. Characterization of monoclonal antibody to DNA.RNA and its application to immunodetection of hybrids. J. Immunol. Methods 89, 123-30 (1986).
26. Jeffet, J. et al. Multimodal single-molecule microscopy with continuously controlled spectral resolution. Biophys. Reports 1, 100013 (2021).
27. Douglass, K. M., Sieben, C., Archetti, A., Lambert, A. & Manley, S. Super-resolution imaging of multiple cells by optimized flat-field epi-illumination. Nat. Photonics 10, 705-708 (2016).
28. Edelstein, A., Amodaj, N., Hoover, K., Vale, R. & Stuurman, N. Computer Control of Microscopes Using μManager. Curr. Protoc. Mol. Biol. 92, 1-17 (2010).
29. Edelstein, A. D. et al. Advanced methods of microscope control using Manager software. J. Biol. Methods 1, 10 (2014).
30. Ovesný, M., Křížek, P., Borkovec, J., Švindrych, Z. & Hagen, G. M. ThunderSTORM: a comprehensive ImageJ plug-in for PALM and STORM data analysis and super-resolution imaging. Bioinformatics 30, 2389-2390 (2014).

### Example 2

### Materials and Methods

### Sample Preparation

Intestinal biopsies were obtained from two patients with ulcerative colitis undergoing routine colonoscopies and two healthy individuals as a control. Biopsies were immediately transferred to the laboratory in complete medium (CM), consisting of RPMI 1640 supplemented with 10% fetal calf serum (FBS) 100 U/ml penicillin, 100 μg/ml streptomycin and 2.5 μg/ml amphotericin B (Fungizone) on ice (to preserve the intact tissue alive). Samples were then washed with sterile phosphate buffer saline (PBS) (Biological Industries) and cultured in CM supplemented with 100 μg/ml gentamicin (Biological Industries) and 0.001% DMSO (Sigma-Aldrich) in an atmosphere containing 5% CO₂ at 37° C. for 18 hours.

For RNA extraction, biopsies were homogenized in ZR BashingBead Lysis Tube (Zymo Research) using a high-speed bead beater (OMNI Bead Ruptor 24). Total RNA was extracted using Trizol® (Invitrogen) according to a standard protocol. RNA concentration and quality were assessed using NanoDrop Spectrophotometer (Thermo Scientific). The 260/280 and 260/230 ratios in all samples were >1.8.

### NanoString Barcode Hybridization and Readout

Two cartridges, each containing the same four samples, were prepared according to the manufacturer's instructions using the nCounter Human Inflammation V2 Panel (NanoString). Briefly, hybridization buffer combined with the codeset of interest is combined with 5 l (400 ng) of total RNA and incubated at 65° C. overnight. To remove all fiducial markers for imaging on the CoCoS system, we extracted all the imaging buffer containing the fiducial markers from one of the reagent plates prior to loading it onto the prep station. Samples were then loaded onto the prep station and incubated under high sensitivity program for 3 hours. Following the prep station, one cartridge was read using NanoString digital analyzer with the high-resolution option, and the other was loaded with fresh fiducial-less imaging buffer and was read using our DeepQR analysis as described herein. The raw barcode counts were output both by DeepQR and digital analyzer in RCC files and were further normalized and processed by the same analysis pipeline as described below.

### Optical Setup

The optical setup was primarily equivalent to the one introduced previously in ref 5, with minor changes in the choice of the emission telescope's lenses.

Excitation: For excitation we used three lasers (Cobolt AB, Sweden) with wavelengths 488 nm (MLD 488, 200 mW max power), 561 nm (Jive 561, 500 mW max power), 638 nm (MLD 638, 140 mW max power). All lasers were mounted on an in-house designed heatsink which coarse aligned their beam heights. Each laser beam was passed through a clean-up filter (LL01-488-12.5, LL01-561-12.5, LL01-638-12.5, Semrock, USA) and expanded to 12.5-20× its original diameter (3×LB1157-A, 3×LB1437-A, Thorlabs, USA). A motorized shutter (SH05, Thorlabs, USA) was used for modulating on/off the solid-state 561 nm laser, while the diode lasers were modulated directly on the laser head. The beams were then combined into a single beam using long-pass filters (Di03-R488-t1-25.4D, Di03-R561-t1-25.4D, Semrock, USA). To homogenize the excitation profile of the sample, the combined beam was passed through an identical setup to the one described in the work of Douglass et al.¹¹. In short, the combined beam was injected into a compressing telescope (AC254-150-A-ML, AC254-050-A-ML, Thorlabs, USA) with a rotating diffuser (24-00066, Süss MicroOptics SA, Switzerland) placed about 5 mm before the shared focal points of the telescope lenses (see FIGS. 1A-D). A series of 6 silver mirrors (PF10-03-P01, Thorlabs, USA) was then used to align the beam into a modified microscope frame (IX81, Olympus, Japan), through two identical microlens arrays (2×MLA, 18-00201, Süss MicroOptics SA, Switzerland) separated by a distance equal to the microlenses focal length and placed inside the microscope frame. The homogenized beam was reflected onto the objective lens (UPlanXApo 60×NA1.42, Olympus, Japan) by a four-band-multichroic mirror (Di03-R405/488/532/635, Semrock, USA). The sample was placed on top of motorized XYZ stage (MS-2000, ASI, USA) with an 890 nm light-emitting diode (LED) based autofocus system (CRISP, ASI, USA), which enabled scanning through multiple fields of view.

Emission: The emitted fluorescence light was gathered by the same objective and transmitted through the multichroic mirror onto a standard Olympus tube lens to create an intermediate image at the exit of the microscope frame. This image was passed through a filter wheel (Sutter Lambda 10-B, Sutter Instruments, USA) with three emission filters: multi-band-filter (FF01-440/521/607/694/809-25, Semrock, USA), 575/15 (FF01-575/15-25, Semrock, USA) or 620/14 (FF01-620/14-25, Semrock, USA). Light was then directed into a magnifying telescope (Apo-Rodagon-N 105 mm, Qioptiq GmbH, Germany and Olympus' wide field tube lens with 180 mm focal length, #36-401, Edmund Optics, USA), with two commercial direct vision prisms (117240, Equascience, France) placed within the infinity space between the lenses and mounted on two motorized rotators (8MR190-2-28, Altechna UAB, Lithuania) controlling the prisms' angles around the optical axis. The final image was acquired on a back illuminated sCMOS camera (Prime BSI, Teledyne Photometrics, USA).

Image acquisition was coordinated using the micro-manager software¹², controlling camera acquisition, laser excitation, XY stage location, and prism rotator angles. The camera and lasers excitation were synchronized using an in-house built TTL controller based on an Arduino® Uno board (Arduino AG, Italy)¹³.

### Image Acquisition

Each of the four sample lanes was fully scanned laterally and imaged, obtaining approximately about 1000 FOV per sample lane. In each one of the FOVs a six image acquisition was taken with specifications according to Table 4:

TABLE 4
Image acquisition parameters.

| | | Emission | Exposure | Laser |
|---|---|---|---|---|
| Excitation | RPA | Filter | time | Intensities |
| All lasers | 178 | multi-band-filter | 300 ms | 638 Laser- |
| simultaneously | | | | 80 mW |
| All lasers | 180 | multi-band-filter | | 561 Laser- |
| simultaneously | | | | 80 mW |
| 638 Laser | 180 | multi-band-filter | | 488 Laser- |
| 561 Laser | 180 | 620/14 (AF594) | | 100 mW |
| 488 Laser | 180 | multi-band-filter | | |
| 561 Laser | 180 | 575/15 (cy3) | | |

The full lane acquisitions were stacked in FIJI¹⁴, resulting in a multi-FOV hyperstack with 6 channels that were input to the deep learning (DL) analysis pipeline.

NanoString nCounter Image Acquisition

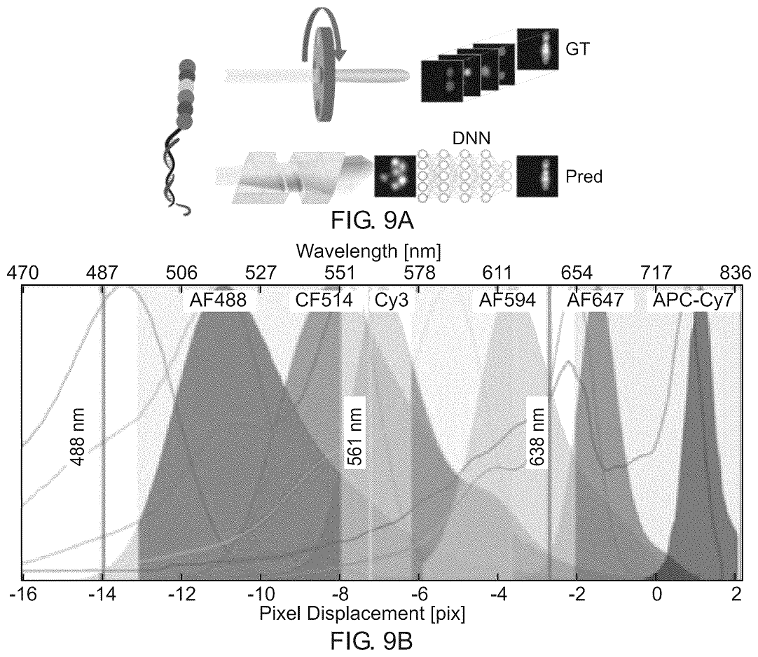

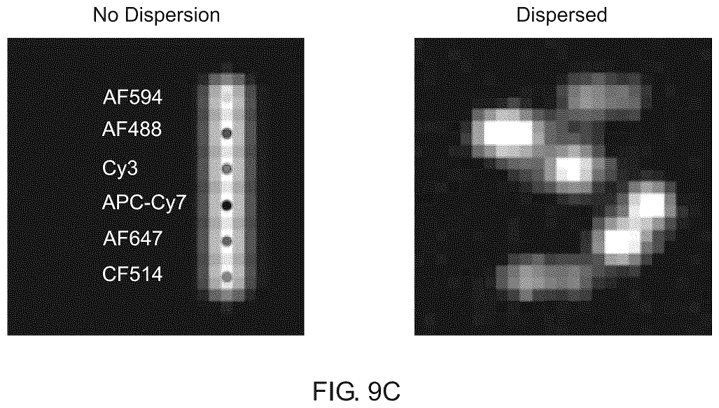

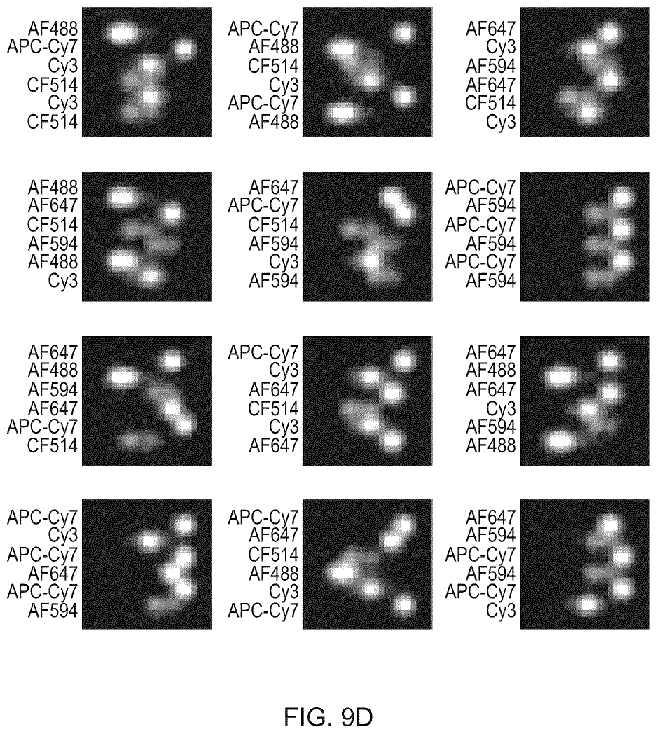

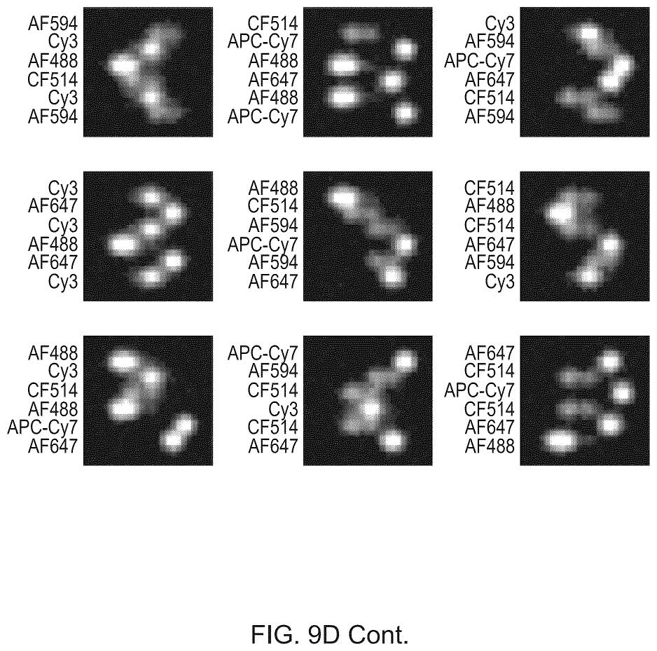

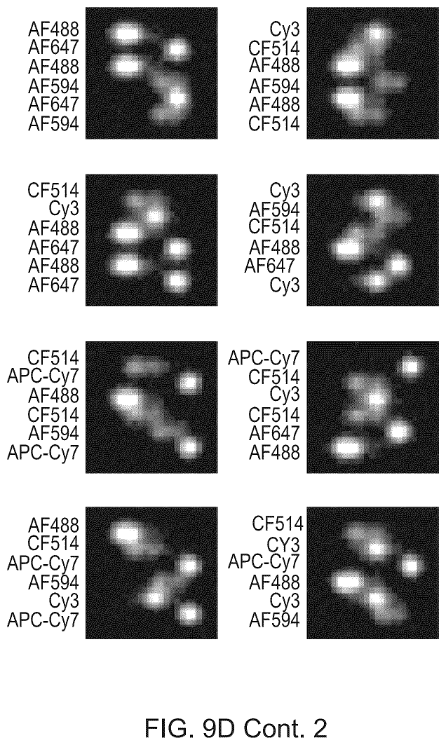

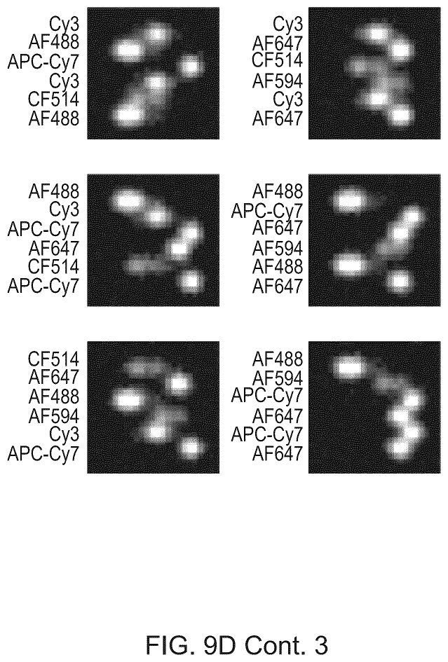

The nCounter experimental assay has been thoroughly described previously⁴ and is given here for comparison completeness. Briefly, the barcoded samples mixed with Tetra-speck micro-spheres (used as fiducial markers) are stretched and immobilized on specialized slides. The slides are scanned and each FOV is imaged four times with different excitations and emission filters (FIG. 9A) to detect the four-colored barcodes sequentially. The Tetra-speck micro-spheres are then used for image registration of the four different color channels and chromatic aberration correction.

### Deep Learning Analysis

Converting the dispersed image stacks to 4-channels non-dispersed multi-FOV hyperstacks was implemented with DL using Tensor-flow and Open-CV packages in Python. The DL architecture used was based on a U-Net architecture⁷, a fully-convolutional encoder-decoder neural network. This architecture is indifferent to the dimensions of the input image and uses skip-connections between the encoder and decoder parts of the network to preserve spatial features encoded in different levels and may have been lost in the encoding process. Each layer consists of two sets of 3×3 convolution filters and non-linear activation layers, succeeded by a 2×2 down-sampling or up-sampling operation for the encoder and decoder parts, respectively. Due to our dispersion-to-color conversion task, we adjusted the original U-Net architecture such that the dimensions of the output image were changed to four channels.

To train the DNN model, we used a full sample lane hyperstack, consisting of 1120 FOVs acquired with the same parameters as in Table 1. The network's input was 1120, single-channel, dispersed FOVs paired with 1120 four-channel FOVs as ground-truth. To artificially increase the data for training, each 1024×1024 pixel² FOV was segmented into 49 overlapping crops of 256×256 pixel² with a 50% overlap between adjacent crops. This dataset was divided into 80% training data, 10% validation data, and 10% test data, and was trained over 200 epochs setting the model's weights to minimize the mean absolute error (MAE) loss. The final model's weights were saved according to the minimal validation loss. To further refine the trained model and improve the distinction between the green (Cy3) and yellow (AF594) dyes, we retrained the network with the same dispersed input paired with the green ground-truth channel only. This produced a second, more accurate model for the green channel. We then used the first model to predict the red (AF647), yellow (AF594) and blue (AF488) channels and the second to predict the green (Cy3) channel and combined their results to create the output four-channel hyperstack. Since our model was trained on 256×256 pixel² patches, for prediction, we divided the 1024×1024 pixel² dispersed FOVs input into 16 patches of 256×256 pixel². After prediction, we stitched the predicted patches to enable visual comparison of the full FOVs. Finally, we used the two learned models trained on this lane to predict the results of the other four sample lanes without additional training, allowing us to extract the full distribution of barcodes from each sample.

To assess the amount of data needed to achieve optimal results, we evaluated our training procedure over smaller subsets of randomly selected FOVs showing the tradeoffs of training on smaller datasets and reducing the number of training epochs. To avoid a non-representative validation subset in training small subsets of FOVs, we first filtered out any out-of-focus or noisy FOVs by applying a set of criteria to the dispersed FOV. Any FOV with mean values >600 ADU, pixels standard deviation values outside 100-600 ADU, or maximal pixel value <3000 ADU was discarded.

### Image Analysis

FOV filtering: Prior to barcodes readout from the hyperstacks, we first removed out-of-focus or noisy FOVs to avoid false barcode readouts. The filtration procedure was carried out in FIJI using the ground-truth hyperstacks as follows:
1. Sum all four hyperstack color channels to produce a grayscale multi-FOV stack.
2. Measure mean pixel value and standard deviation of each FOV.
3. Filter out FOVs with mean values outside the 500-700 ADU range or standard deviations outside the 50-1000 range.

Barcode detection and cropping: to reliably compare the barcode readout between the ground-truth and prediction hyperstacks, we wanted to compare readouts from the same locations in both hyperstacks. Therefore, we detected the barcodes on the ground-truth hyperstack by applying the multi-template matching plugin in FIJI¹⁵ on the color-channel-summed ground-truth. This allowed us to localize all barcode-like features in the ground-truth stack and to extract a 20 by 9 pixels crops from each of these locations both in the ground-truth and prediction 4-channel hyperstacks. The procedure was carried out as follows:
1. Sum all four hyperstack color channels to produce a grayscale multi-FOV stack.
2. Apply the Multi-Template Matching plugin with a cropped grayscale barcode template.
3. Split the color channels of both ground-truth and prediction hyperstacks.
4. Use FIJI ROI manager Multi-crop function to crop the same barcode detections from all channels of both ground-truth and prediction hyperstacks.
5. Merge the cropped barcode stacks channels to receive multi-barcode hyper-stacks, allowing a location-based comparison between barcodes.

### Weighted Spectral Centroid Calculation

The spectral difference between the Cy3 and AF594 emissions was calculated using the weighted centroid of each color's intensity profile. To calculate the weighted centroids, we first extracted intensity profiles for each marker in all spectral dispersions by averaging the intensity along a 3 pixel-wide line centered around each marker for all dispersions. Then we subtracted the background value from each profile, and calculated the weighted centroid by the following equation:

$$WC = \frac{\sum {x}_{i} \cdot {I}_{i}}{\sum {I}_{i}}$$

Where WC stands for weighted centroid in pixels, xᵢ is the pixel location along the line profile, and Iᵢ is the intensity at the i-th pixel. Finally, each of the markers' profiles was aligned to the weighted centroid locations in their respective no dispersion profile (RPA=180°), setting them as the reference location for spectral displacement for each marker.

### Bleed-Through Correction and Barcode Readout

One major issue we had to overcome in resolving the correct color sequence of the barcodes was the bleed-through from the green (cy3) channel to the yellow (AF594) channel. Due to the spectral properties of these dyes and our excitation wavelength, an emission filter-based separation of the two dyes was insufficient, and post-acquisition correction of the barcode images was employed. To readout the barcodes color sequence from the cropped barcode stacks, we used an in-house readout Matlab code that follows these steps:
1. Import the barcode image stacks using built-in Matlab functions for tiff file reading.
2. Create profiles along both barcode axes to extract initial peaks locations in all channels using the built-in Matlab function findpeaks.
3. Peaks along the barcode axis that are wider than the nominal PSF were fitted by 2 gaussian model to resolve overlapping PSFs (which might occur due to small focus deviations).
4. Localize the peaks in the red (AF647), green (Cy3) and blue (AF488) channels by 2-d gaussian fits at the initial positions using FastPsfFitting Matlab functions written by Simon Christoph Stein and Jan Thiart and which are available on Matlab file exchange. The peak localization of the yellow (AF594) channel is done after bleed-through correction.
5. To characterize the bleed-through from the green to the yellow channel, find all complete barcodes containing six localizations without the yellow markers and at least one localization in the green channel.
6. Estimate the mean parameters for the bleed-through PSF according to the green marker PSF: x-shift, y-shift, intensity ratio, and standard deviation ratio.
7. Use these parameters to subtract simulated bleed-through PSFs from the yellow channel images according to the green channel localization results and then localize the yellow markers on the bleed-through corrected images.
8. Combine all channels readout and perform quality check (QC) according to known barcode limitations: six markers per barcode, adjacent markers should have different colors, minimal separation between adjacent markers along the barcode's axis, and maximal shift between markers along the perpendicular axis. Barcodes that did not meet the QC limitations were discarded.
9. The final barcode readout is then organized in a table and enumerated according to its crop number in the barcodes crop stack for the following ground-truth to prediction barcode readout comparison.

### Barcode to Gene Counts Conversion

After reading the color code of the barcodes, a conversion to the corresponding gene names was performed using the RLF file provided with the nCounter dataset, containing the code-set conversion between color-code and gene identity.

The total barcode reads of ground-truth and prediction tables were counted and assigned to their relevant genes. Only barcode reads corresponding to the nCounter code-set were kept throwing away all false detections.

### Gene Expression Count Normalization

In order to normalize the gene counts, the output gene counts tables from the barcode readout were converted to RCC files, which were further processed by the NanoString nSolver 4.0 software. We normalized all experimental results together: the nCounter, ground-truth and prediction RCC files in the same nSolver experiment. The normalization was performed according to the standard protocol with thresholding according to the geometric mean of negative controls, normalization according to the geometric mean of positive controls A-E (excluding POS_F due to a higher limit of detection in our custom detection analysis) and the standard CodeSet housekeeping genes normalization (according to the geometric mean of CLTC, GAPDH, GUSB, HPRT1, PGK1 and TUBB genes). The normalized results were exported as a text file for further analysis of the gene expression in Matlab.

### Most Differentially Expressed Genes

To calculate the most differentially expressed genes, the normalized expression results from the nSolver were processed with the “rnaseqde” Matlab function to give an adjusted p-value and log 2 fold changes. The four samples were processed independently for each acquisition method (prediction, ground-truth and nCounter) and the three resulting gene expression tables were filtered for adjusted p-values smaller than 0.05. The tables were reordered according to gene fold-change scores, extracting the top 20 most differentially expressed genes in each one of the acquisition methods. For comparison, the same analysis was performed using ROSALIND® barcode counts normalization and its differential expression analysis, resulting in slightly different results mainly due to different approaches for P-value calculation as described in the next section.

### ROSALIND® NanoString Gene Expression

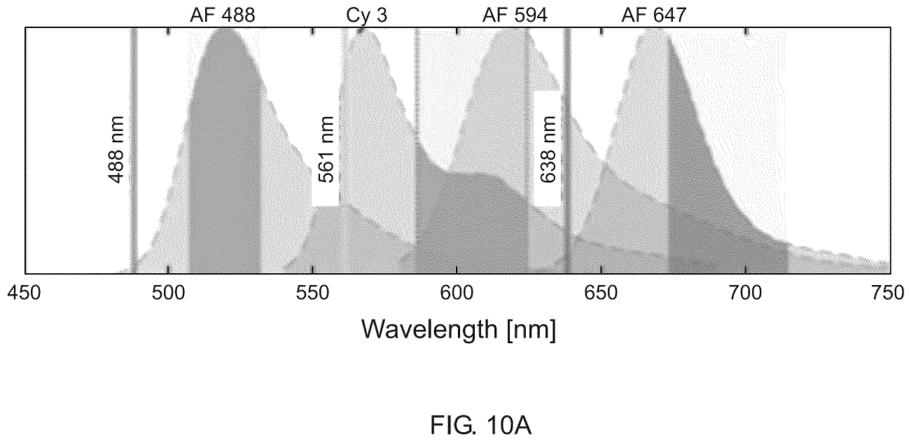

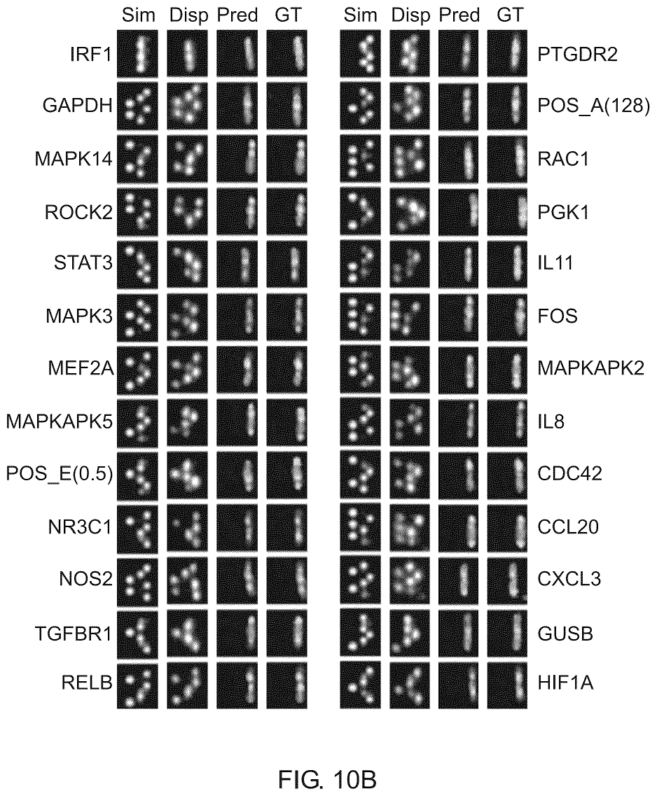

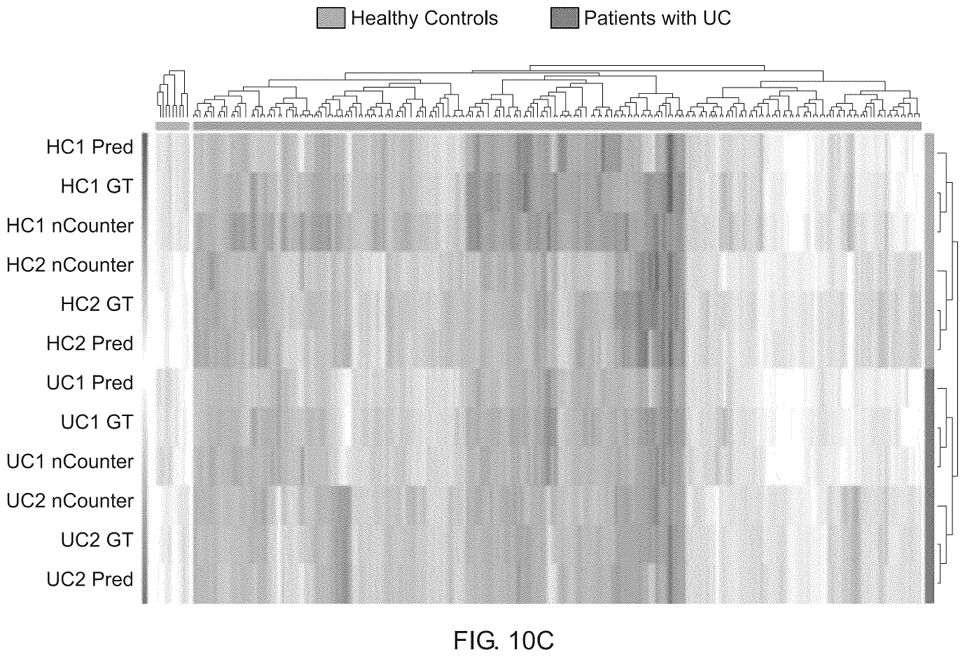

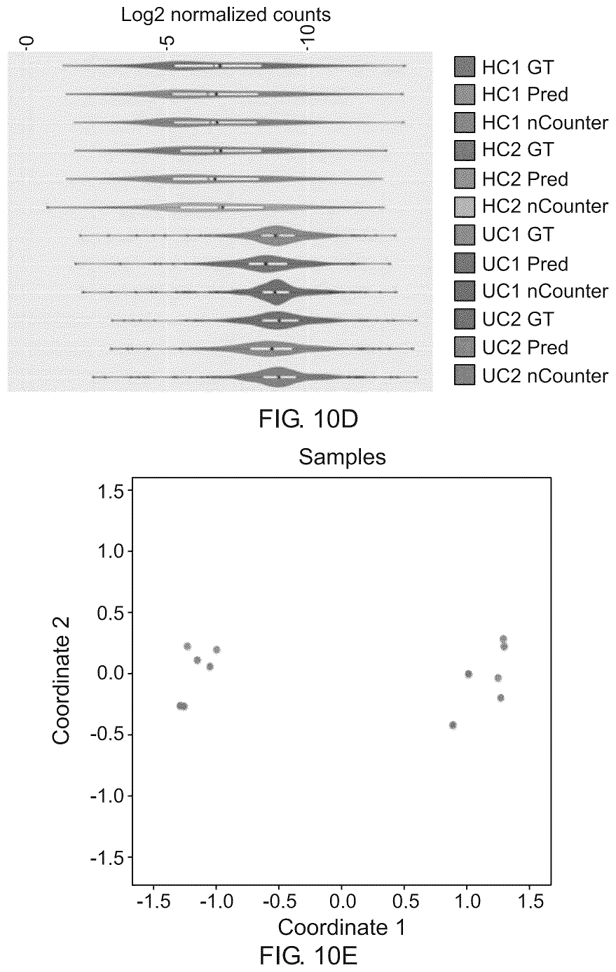

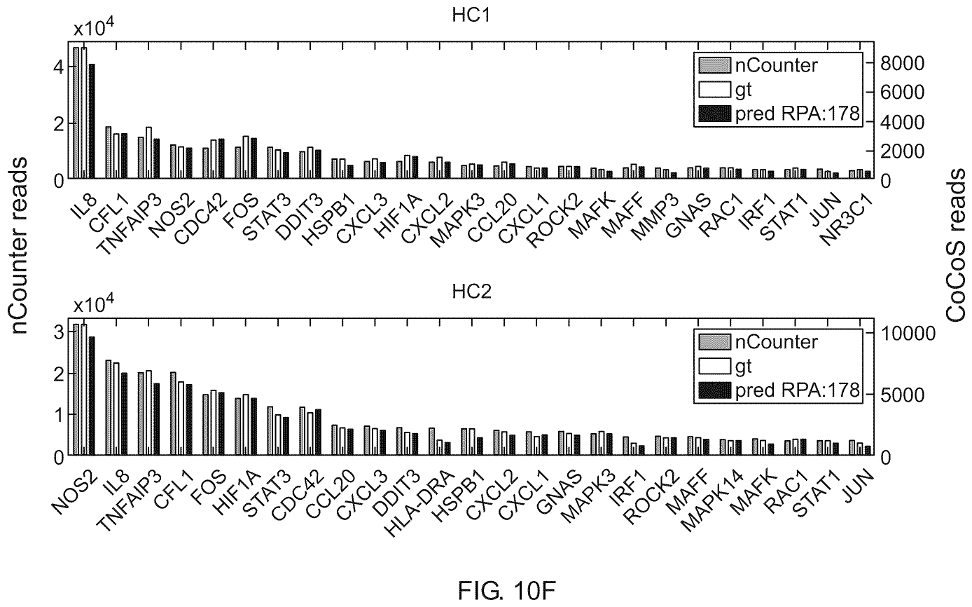

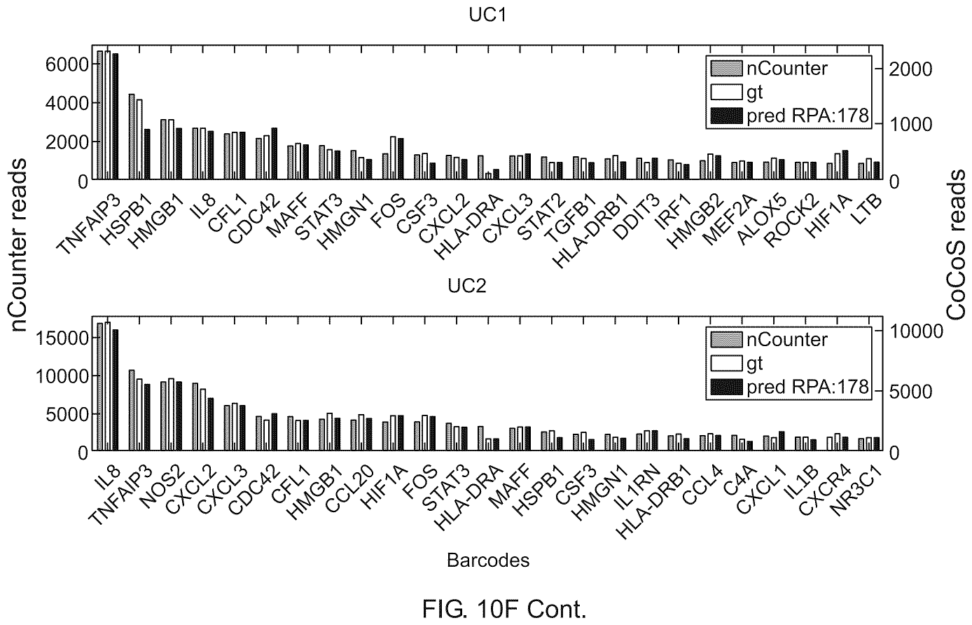

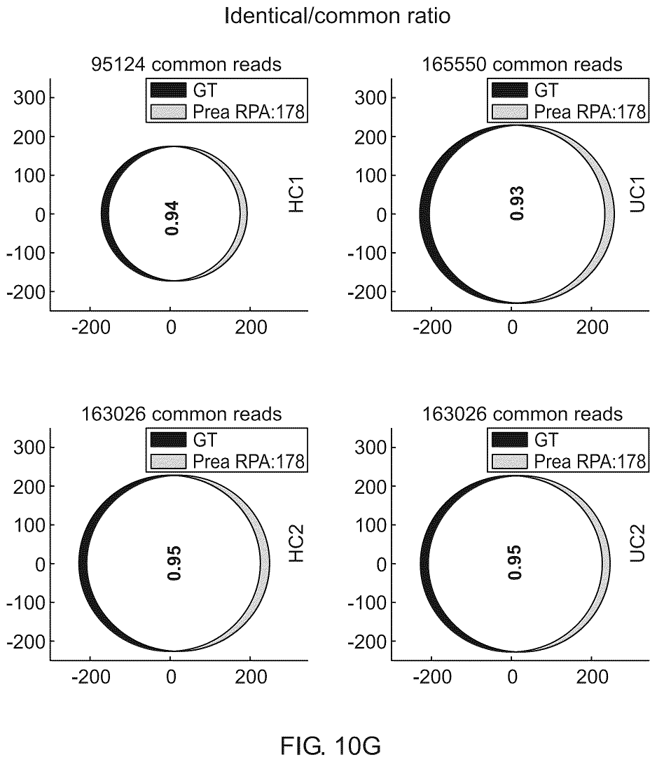

For creating the gene expression heatmap (FIG. 10A), violin plots (FIG. 10B), and sample MDS plots (FIG. 10C), data was analyzed by ROSALIND®, with a HyperScale architecture developed by ROSALIND, Inc. (San Diego, CA). Normalization, fold changes and p-values were calculated using criteria provided by NanoString. ROSALIND® follows the nCounter® Advanced Analysis protocol of dividing counts within a lane by the geometric mean of the normalizer probes from the same lane. Housekeeping probes to be used for normalization are selected based on the geNorm algorithm as implemented in the NormqPCR R library¹⁶. Fold changes and pValues are calculated using the fast method as described in the nCounter® Advanced Analysis 2.0 User Manual. P-value adjustment is performed using the Benjamini-Hochberg method of estimating false discovery rates (FDR). Clustering of genes for the final heatmap of differentially expressed genes was done using the PAM (Partitioning Around Medoids) method using the fpc R library¹⁷ that takes into consideration the direction and type of all signals on a pathway, the position, role and type of every gene, etc. To effectively compare the same four samples across the three analysis methods (GT, Prediction and NanoString's nCounter), we used Rosalind's covariate correction analysis with the detection method as a hidden covariate.

### Heatmap Analysis

The two-color heatmap (FIG. 10A) represents the mean-subtracted normalized log 2 expression values, i.e., for each gene, the average of the log 2 normalized expression is taken and subtracted from each sample's expression.

### Barcode Detection Performance Analysis

Ground-truth and prediction barcode detection performances were compared to one another and to nCounter readout of the same samples in a different experimental run.

Ground-Truth Vs. Prediction Comparison

To compare barcode detection performance between ground-truth and prediction, we first filtered only the “common barcode reads” where both the ground-truth and prediction obtained valid barcode detection (barcodes that passed our filtering QC). Out of these common valid reads, we compared each barcode readout and counted the number of identical reads in both stacks (FIG. 10E). The Venn diagram representation of the ratio of identical barcodes out of all common barcodes was generated using Darik Gamble's ‘venn’ Matlab script available online from Matlab Central file exchange.

### Histogram Comparison of Raw Barcode Counts

To compare the nCounter results to our readout, all RCC files obtained from the NanoString digital analyzer were first exported to csv files using the nSolver 4.0 software. The barcodes obtained from our readout were counted according to their color sequence using the built-in Matlab function ‘histcounts’. Finally, to assess our readout pipeline, the raw barcode counts from the nCounter were compared to our readout from the ground-truth and prediction stacks by plotting the 25 most abundant endogenous genes in histograms (FIG. 10D).

### Error Analysis of Predicted Barcodes Readout

To follow the origin of DeepQR prediction errors, we analyzed the eligible barcode reads (i.e., reads that passed QC) that mismatched between the ground-truth and prediction. We compared the markers between ground-truth and prediction readouts for each mismatched barcode and registered all discrepancies. The distribution of read errors per marker color was calculated by considering only barcodes with a single marker mismatch to avoid over-representation of errors due to missed or falsely inserted markers (which create a permutation of the markers in the barcode read and therefore excess error detections).

### Fiducial Markers Excluded Area Calculation

If a barcode overlaps with a fiducial marker, it will not be read correctly in the NanoString pipeline and will be discarded. To quantify the percentage of inaccessible FOV due to fiducial markers in the standard NanoString pipeline, we used 10 non-dispersed FOV simultaneously excited by all three lasers. We used intensity threshold to locate all fiducials and created a fiducial binary mask using FIJI's¹⁴ IsoData stack to binary conversion. As each barcode takes up an area of about 5×15 pixels² we convolved the binary fiducial mask with a 5×15 all-ones matrix to produce a binary estimate of the excluded area. The percentage of FOV excluded by fiducial markers was calculated as the mean value over an area of 600×600 pixels² at the center of 10 convolved binary images resulting in 9.0±1.1%.

### PSFs Simulations

All simulations were performed by a home-built Matlab code. Here we provide a short description of the pipeline:
1. Excitation and emission spectra of 6 commercial dyes together with either of our multi-band emission filters (four-notch filter (NF03-405/488/561/635E, Semrock, USA) for the 6-color barcode simulations or 5-band (FF01-440/521/607/694/809-25, Semrock, USA) for the 4-color barcodes simulations) were downloaded from Semrock's SearchLight™ spectra viewer. Each of the dyes spectrum was multiplied with the filter's spectrum to produce the actual spectrum imaged on our camera.
2. The wavelength to pixels displacement calibration curve of our CoCoS setup (which was calculated previously⁵) was adjusted according to the experimentally used RPA by multiplying the entire curve by sin((180-RPA)/2).
3. Barcodes dye combinations were then simulated by converting each dye's spectrum into a diffraction limited dispersed image. This was done by assigning a gaussian with unity amplitude and 1.2 pixel standard deviation to each wavelength in the emission spectrum. Each gaussian was displaced according to the RPA-adjusted displacement curve and summed together with other gaussians. Finally, the total summed intensity of all gaussians was normalized to unity and multiplied by an excitation efficiency factor which was calculated by the excitation spectrum value (fractions only) at the excitation laser wavelength.
4. This process was repeated for the randomly selected dyes at the six barcode locations and all images were summed to provide the barcode's spectral image.
5. To further resemble the experimental images a noise model was added to all simulated spectral PSF images using the imnoise function in Matlab. The noise model used in this work was a sum of a poisson distributed shot-noise and gaussian noise with a constant mean of 0.3 and 0.000625 variance.

### Results

Transcriptome analysis is a powerful tool for exploring physiological responses to environmental exposures, external stimuli, and various disease states. RNA sequencing is able to characterize the full RNA content of a sample but necessitates reverse transcription and PCR amplification which introduces bias to quantitative expression analysis. An outstanding goal in single-molecule analysis is to capture the full transcriptome (about 20,000 protein-coding genes, according to GENCODE v.34) from a native sample without PCR amplification. Here, we introduce DeepQR, an optical method that generates thousands of unique molecular identifiers for RNA targets, using spectral imaging combined with machine learning-based image registration. DeepQR exploits the visible spectrum more efficiently than conventional filter-based microscopy, allowing for combinatorial color multiplexing. By introducing minute spectral changes to the detected optical point spread function (PSF), it allows distinguishing between spectrally similar fluorophores in the same color channel of the microscope. Thus, a conventional scientific monochrome camera can simultaneously record more than six different colors in the visible spectrum with a single snapshot.

To demonstrate DeepQR capabilities, we refer to the commercially available fluorescent barcodes from NanoString Inc. These are RNA specific hybridization probes that report on the identity of the captured RNA target via a linear DNA barcode composed of four fluorescent colors arranged in various combinations at six positions along the linear barcode (FIG. 9A).

Introducing more colors to the barcode palette significantly increases the number of unique RNA targets. However, standard optical setups do not allow additional color channels without considerable spectral cross-talk. We use tunable spectral imaging to disperse the barcode emission spatially, allowing the introduction of additional colors independent of conventional filter channels. Effectively, the linear color barcode is transformed into a two-dimensional QR-code-like image, with color encoded perpendicular to the barcode axis. Spectral PSF simulations show that two additional flourophores may be detected with the same filter configuration, significantly enriching the barcode pallet (FIG. 1B). Such six-color barcodes resolve 18,750 RNA targets, 20-fold more than the original four-color NanoString barcodes (FIGS. 1C and 1D). In addition, such a configuration allows acquiring all data channels with a single snapshot, eliminating the need for sequential multi-color imaging and alignment.

Using the same simulations combined with experimental validation, we benchmarked our approach against the current state of the art in single molecule transcriptomics, the NanoString nCounter system. The nCounter gene expression platform is a broadly used optical method for direct single-molecule RNA expression quantification (Geiss, G. K. et al. Direct multiplexed measurement of gene expression with color-coded probe pairs. Nat. Biotechnol. 26, 317-325 (2008). Counting the various barcodes determines the gene expression levels with high accuracy and sensitivity at the single molecule level. The nCounter system uses four-color channels to sequentially acquire the four-color NanoString barcodes (FIG. 9A top), resulting in 2,220 separate acquisitions per sample. For direct comparison, we designed DeepQR to simultaneously image the four-color barcodes with a single acquisition (FIG. 10A), therefore, DeepQR requires only 555 acquisitions in order to resolve the same sample. In addition, we use a single filter with only three color channels for resolving all four colors, showcasing our ability to resolve multiple fluorophores in the same spectral window.

DeepQR harnesses the synergetic combination of deep neural networks (DNN) analysis with continuously controlled spectral-resolution (CoCoS) imaging scheme. In CoCoS two direct-vision prisms introduce controlled spectral dispersion in a single axis such that all colors can be imaged simultaneously with a single snapshot (FIG. 9A). Tuning the dispersion is crucial for minimizing the spectral footprint of the barcodes, allowing to maximize the density of resolved single molecules in the FOV without introducing overlaps between molecules. Moreover, the simultaneous acquisition with a single emission filter also removes the need for fiducial markers, used in nCounter to align the different color channels. This frees up about 9% of the field of view (FOV) allowing even higher barcode densities and better throughput. DNN are perfect complement to CoCoS as they can recognize even minute spectral changes introduced to the PSF, therefore allowing to further minimize the dispersion required for efficient color classification. The spectrally dispersed images are decoded in DeepQR by a U-Net architecture DNN 7 (see methods) that reconstructs each dispersed FOV into a non-dispersed multi-color channel image.

We trained the U-Net with 1120 matched pairs of dispersed and non-dispersed four-channel ground-truth FOVs, imaged from a single nCounter sample. The ground-truth images were acquired by sequentially switching the excitation lasers and emission filters to register each color separately. In contrast, the dispersed images were acquired in a single frame through a multi-band emission filter (FIGS. 10A and 10B). Next, the trained network was applied to reconstruct dispersed images of different samples without additional training. With this pre-trained network DeepQR was able to resolve all four barcode colors using a single acquisition per FOV.

Notably, two of these colors: the green Cy3 and yellow Alexa Fluor 594 dyes, were spectrally overlapping in the same channel of our system (FIG. 10A). With DeepQR's classification, we could resolve them with sub-pixel spectral displacement difference, allowing better color classification resolution than achievable with standard spectral fitting. This differentiation showcases DeepQR's ability to resolve many more color combinations than nCounter with the same spectral channels (FIGS. 9B, 9C and 9D).

To benchmark the performance of DeepQR, we performed a clinical gene expression experiment registering the differential expression signature of patients with ulcerative colitis (UC) using the commercial NanoString inflammation gene expression panel. A differential expression pattern in inflammatory genes has been observed between healthy and inflamed intestines. Our experiment compared four RNA samples derived from intestinal biopsies taken from two UC patients and two healthy controls (see methods). Our network was trained to minimize the mean-absolute-error (MAE) between ground-truth and network predictions. Then, we assessed the network's performance by comparing the predicted barcodes with the ground-truth. Finally, the experimental end-products, i.e., the barcode expression count distributions, were compared to those achieved by the nCounter system. We directly compared ground-truth and network prediction barcodes by extracting the same barcodes in both datasets and performing pairwise comparisons. This comparison allowed us to assess missed or erroneous network classifications. One challenge in exciting multiple color markers with a single laser is significant bleed-through; in our case, the green dye to the yellow emission channel during ground-truth acquisition. We addressed the bleed-through by registering the yellow bleed-through component of the green markers, establishing a global bleed-through correction function to the yellow channel. We note that even barcodes solely composed of yellow and green markers imaged through the same spectral band, such as the one for the IRF1 gene, were correctly classified (FIG. 10B, top left).

The color order of the cropped barcodes was used to produce the global count distributions of both ground-truth and network prediction barcodes. Despite the sub-optimal efficiency of our barcode readout process, we obtained 92-95% concordance between ground-truth and prediction (FIG. 10E). We then compared out results to the nCounter readout (FIGS. 10C-F).

The raw barcode count distributions obtained by DeepQR predictions show similar results compared to the standard 4-color imaging of both ground-truth and nCounter (FIG. 10F). DeepQR's barcode predictions comparison with the corresponding 4-channel ground-truth readout show 92-95% agreement (FIG. 10E). The fraction of unsuccessful predictions was predominately attributed to an error in either green or yellow classification and are a joint result of both neural network prediction errors and our partially inefficient post-process bleed-through correction. Even with these prediction errors, the DeepQR results align very well with the results obtained by the commercial nCounter system, and the gene expression distributions of the four samples are well reconstructed by our method.

Overall, our analysis correctly classifies the UC patients according to their gene expression patterns (FIGS. 10A-C) and reproduces 17 out of the 20 most differentially expressed genes in the nCounter analysis (compared to 18 out of 20 in the ground-truth analysis. This concordance validates that the present analysis could be used clinically to assess unbiased gene expression profiles.

Using DeepQR with a pre-trained DNN, all RNA species in the NanoString panel are resolved with a quarter of the frames per FOV resulting in more than 4-fold faster acquisition speeds. This allows to significantly increase turnaround times for gene expression analysis, crucial for clinical point-of-care analyses. Furthermore, DeepQR allowed resolving the four-color NanoString barcodes with three spectral channels and excitation sources increasing the target multiplexing capabilities by 10-fold. In future experiments six or seven-color barcodes could be used to achieve full-transcriptome analysis at single-molecule sensitivity.

In conclusion, we demonstrate a novel approach for fast and efficient color registration and multiplexing at the single molecule level. DeepQR is compatible with demanding multi-color single-molecule applications such as classifying the barcodes utilized by NanoString, enabling immediate utilization in applications requiring high multiplexing capabilities. DeepQR provides a traditional multi-channel output compatible with standard downstream analyses and supports multiplexing of spectrally overlapping colors while completing the entire color acquisition pipeline without exchanging filters and at a fraction of the standard acquisition time.

Specifically, DeepQR recorded the NanoString inflammation panel in less than a quarter of the standard acquisition time and without any fiducial markers, achieving results highly correlated with the nCounter system. Beyond the similarity in gene count distributions, the majority of differentially expressed genes were identical in both methods.

This Example demonstrated that DeepQR resolves four-color barcodes with 92-95% accuracy using only three spectral channels with two of the four colors spectrally overlapped and excited with the same laser. This resolution provides 972 distinctly resolvable barcodes compared to 96 barcodes with conventional filter-based three-channel microscopy. Consequently, this could be used for addressing higher degree multiplexing sorely needed in single-molecule transcriptome analysis. Introducing a fifth color to the NanoString barcodes, which can already be implemented within the existing four nCounter spectral channels, extends the palette of available barcodes to 5,120, allowing the classification of all microRNAs (about 2,600) and a panel of selected genes. However, with three excitation lasers and the current optical setup DeepQR can resolve up to seven distinct colors, providing 54,432 unique barcode combinations and potentially enabling full single-molecule transcriptome profiling.

### REFERENCES FOR EXAMPLE 2

1. Melé, M. et al. The human transcriptome across tissues and individuals. Science (80-.). 348, 660-665 (2015).
2. Frankish, A. et al. GENCODE reference annotation for the human and mouse genomes. Nucleic Acids Res. 47, D766-D773 (2019).
3. Eastel, J. M. et al. Application of NanoString technologies in companion diagnostic development. Expert Rev. Mol. Diagn. 19, 591-598 (2019).
4. Geiss, G. K. et al. Direct multiplexed measurement of gene expression with color-coded probe pairs. Nat. Biotechnol. 26, 317-325 (2008).
5. Jeffet, J. et al. Multimodal single-molecule microscopy with continuously controlled spectral resolution. Biophys. Reports 1, 100013 (2021).
6. Hershko, E., Weiss, L. E., Michaeli, T. & Shechtman, Y. Multicolor localization microscopy and point-spread-function engineering by deep learning. Opt. Express 27, 6158 (2019).
7. Ronneberger, O., Fischer, P. & Brox, T. U-Net: Convolutional Networks for Biomedical Image Segmentation, in 234-241 (2015). doi:10.1007/978-3-319-24574-4_28
8. Song, K.-H., Dong, B., Sun, C. & Zhang, H. F. Theoretical analysis of spectral precision in spectroscopic single-molecule localization microscopy. Rev. Sci. Instrum. 89, 123703 (2018).
9. Ben-Shachar, S. et al. Gene expression profiles of ileal inflammatory bowel disease correlate with disease phenotype and advance understanding of its immunopathogenesis. Inflamm. Bowel Dis. 19, 2509-2521 (2013).
10. Haberman, Y. et al. Ulcerative colitis mucosal transcriptomes reveal mitochondriopathy and personalized mechanisms underlying disease severity and treatment response. Nat. Commun. 10, (2019).
11. Douglass, K. M., Sieben, C., Archetti, A., Lambert, A. & Manley, S. Super-resolution imaging of multiple cells by optimized flat-field epi-illumination. Nat. Photonics 10, 705-708 (2016).
12. Edelstein, A., Amodaj, N., Hoover, K., Vale, R. & Stuurman, N. Computer Control of Microscopes Using Manager. Curr. Protoc. Mol. Biol. 92, 1-17 (2010).
13. Edelstein, A. D. et al. Advanced methods of microscope control using Manager software. J. Biol. Methods 1, 10 (2014).
14. Schindelin, J. et al. Fiji: An open-source platform for biological-image analysis. Nat. Methods 9, 676-682 (2012).
15. Thomas, L. S. V. & Gehrig, J. Multi-template matching: A versatile tool for object-localization in microscopy images. BMC Bioinformatics 21, 1-8 (2020).
16. Perkins, J. R. et al. ReadqPCR and NormqPCR: R packages for the reading, quality checking and normalisation of RT-qPCR quantification cycle (Cq) data. BMC Genomics 13, 1-8 (2012).
17. Hennig, C. CRAN—Package fpc. 12/06/2020 (2020). Available at: www(dot)cran.r-project(dot)org/web/packages/fpc/index.html. (Accessed: 20 Jun. 2022)

### Example 3

### Machine-Learning-Based Single-Molecule Quantification of Circulating MicroRNA Mixtures

This Example presents a technique for multiplexed single-molecule detection and quantification of a selected panel of miRs. The proposed assay optionally and preferably does not depend on sequencing, requires less than one ml of blood, and provides fast results by direct analysis of native, unamplified miRs. This is enabled by a novel combination of compact spectral imaging together with a machine learning based detection scheme that allows simultaneous multiplexed classification of multiple miR targets per sample. The proposed end-to-end pipeline (see FIG. 14) is time efficient and cost-effective. The technique is benchmarked with synthetic mixtures of three target miRs, showcasing the ability to quantify and distinguish subtle ratio changes between miR targets.

MicroRNA (miRs) are evolutionarily conserved, 18 to 25 nucleotide-long noncoding RNAs that regulate the translation of messenger RNA (mRNA)^{1,2}. MiRs regulate the transcription of up to 60% of all human protein-coding genes and are therefore crucial for cellular function³. Aberrant miR levels reflect the physiological state of cancer cells and correlate closely with tumor origin and stage⁴. miRs are highly present in circulation, are protected from RNases digestion by extracellular vesicles (EVs) or protein-binding, and are tightly related to oncogenesis, making them promising candidates for biomarkers⁵⁻⁹. Expression analyses of miRs circulating in blood emerge as promising and complementing clinical tools for early molecular diagnostics and follow-up¹⁰. Relative expression level signatures of small panels consisting of <10 targets of carefully selected miRs have significant diagnostic and prognostic power^{7,11,12}. Such disease-specific panels are already well established in the literature^{7,13} and dedicated databases¹⁴⁻¹⁶.

Current mainstream methodologies for quantifying miR expression are not optimized nor designed for the simultaneous quantification of several miR targets¹⁷⁻²⁰. Known methodologies for miR expression quantification such as quantitative reverse transcriptase polymerase chain reaction (qRT-PCR), RNA sequencing (RNAseq), and microarrays²⁰, rely on PCR amplification for analysis. Due to their short sequence length, miRs are not easily amplified by PCR, introducing bias into such expression analysis^{21,22}. The inventors realized that each of these methods has its drawbacks when quantifying small panels of miRs: qRT-PCR has limited multiplexing capabilities such that analyzing the expression of more than three miR targets often requires extensive optimizations and validations and is limited in sample throughput²³. Known RNAseq requires non-specific sequencing of all small RNAs and therefore suffers from long turnaround times and relatively high costs per target when small panels of targets are needed. Furthermore, due to the large abundance variation between miR targets in body fluids²⁴, deep sequencing is required for expression profiling of rare miRs. Microarrays are good for multiplexing a large number of targets at relatively low costs but suffer from low sensitivity and specificity and are difficult to use for absolute quantification^{19,25-27}.

Native miR detection mitigates these biases and is currently possible with the commercial NanoString system²⁸, however, the Inventors found that this solution is more suitable for panels consisting of hundreds of miRs and is generally excessive and expensive for panels of several miRs of interest²⁹.

Recent studies have demonstrated the capability to optically detect miRs at single-molecule resolutions³⁰⁻³³. Nevertheless, the Inventors found that their multiplexing capabilities are currently limited to two miR targets simultaneously. In clinical applications such as diagnostics and follow-up, there is a need to evaluate miR panels consisting of multiple targets in a fast, cost-effective, and sensitive manner.

This Example introduces miR Analysis by spectral Classification LEarning, referred to below as miRACLE. This technique allows a multiplexed single-molecule detection and profiling, and is particularly useful for small panels (e.g., up to 11 disease-associated miRs).

The miRACLE's pipeline combines several components that are presented below in greater detail. These components comprise (i) capturing targeted miRs by complementary DNA probes labeled with a distinct fluorophore-pair; (ii) specific miR targets immobilization using anti-RNA:DNA hybrid antibody³⁴ and subsequent washing of excess probes and background; (iii) compact spectral imaging using CoCoS microscopy³⁵; and (iv) a dedicated image processing tool for detection and classification of all miR targets at single-molecule sensitivity.

### Results

The miRACLE pipeline allows to isolate and quantify only the miR targets relevant for disease-specific diagnosis. This focused detection allows for increasing the signal to noise ratio (SNR) by reducing background noise, enhancing the dynamic range of the method, and reducing experimental costs (see Table 6, below). This goal can be achieved in two sequential stages. First, specific miR targets are captured and tagged using DNA capture-reporter probes composed of about 30 base pairs long sequences and labeled with unique combinations of two fluorophores. These probes are designed to complement specific miR targets (FIG. 11A), offering target recognition specificity with single nucleotide sensitivity^{31,33}. Thus, upon hybridization with their targets, each miR has a unique spectral signature acting as a “spectral barcode” which discloses their identity. When required, the target recognition specificity can be further improved by implementing locked nucleic acids (LNA) in the probe design^{30,36}.

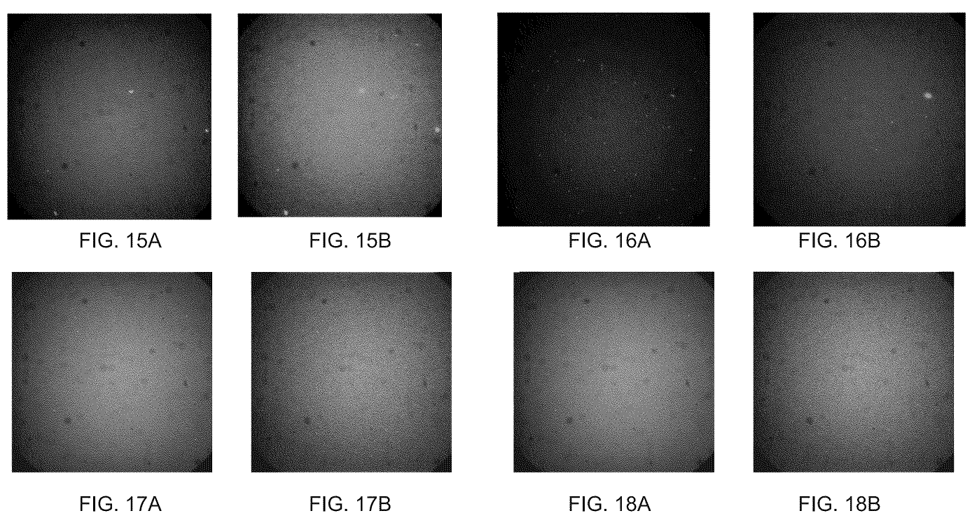

In the following stage, selective capturing and immobilization of the DNA:RNA hybrids are used to isolate the targets from the non-hybridized probes and any auto-fluorescing molecules in the RNA-extract. DNA:RNA hybrids that correspond to hybridized miR targets are specifically captured on microscope coverslips functionalized with a monoclonal Anti-DNA-RNA hybrid [S9.6] antibody^{31,34}, allowing for the selective imaging of only the target miRs (FIG. 11B). After capturing the miR-reporter constructs on the surface, subsequent washes remove all excess unhybridized reporter probes thus significantly reducing background noise. This capture process eliminates the non-specific binding of reporter probes, as was thoroughly validated previously^{31,34} and in control experiments (see FIGS. 15A-18B). As a result, this approach allows a sensitive readout of the relevant targets.

In order to validate the miRACLE concept, three synthetic miR targets were used at physiological concentrations³⁷ and their corresponding DNA probes (sequences are provided in supplementary note 2). First, the three miRs were hybridized each with its complementary probe and were immobilized separately on three different surfaces. These experiments provided a dataset of distinguished probes for training the imaging, detection, and classification process. Next, the three probes were hybridized in two separate mixtures containing all three miR targets. The mixed samples were mixed together at volumes ratios of 1:1:1 and 2:5:3 for miR-15b-5p: miR-155-5p: miR-126-3p respectively. These mixture experiments are used to benchmark the miRACLE pipeline.

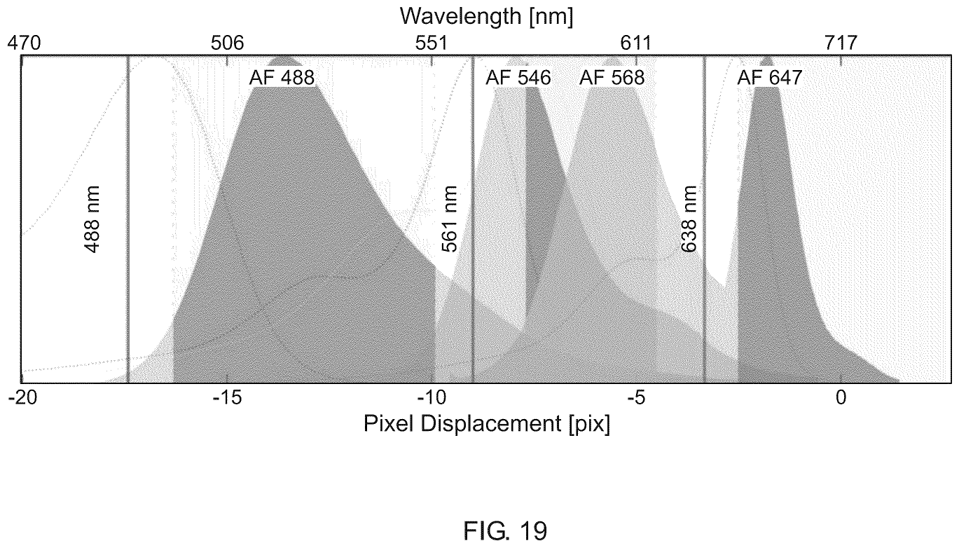

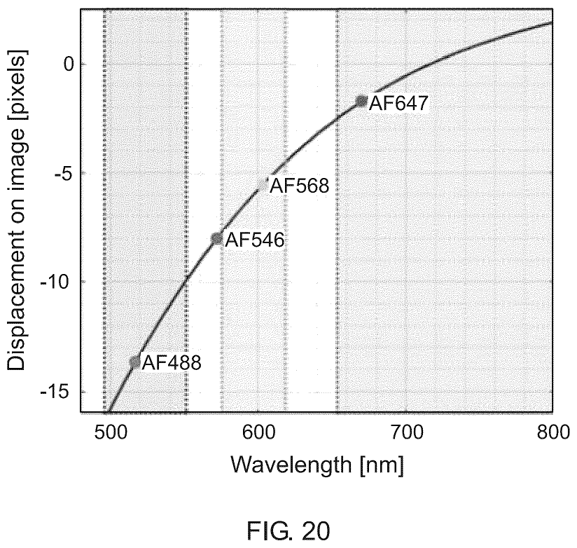

The third stage in the miRACLE process is reading out the spectral barcodes of the miR targets. To this end, the surface-immobilized miR:reporter hybrids with their unique fluorophore-pair combinations are imaged and resolved by miRACLE's compact spectral imaging module based on the previously introduced CoCoS system³⁵. The two fluorophores on each probe are positioned at distances much smaller than the diffraction limit (all probes are about 30 bp in length which are equivalent to about 10 nm) and therefore are captured in the image at the same physical location. To distinguish between the overlapping fluorophores, they were spectrally dispersed using two prisms (FIG. 11C top), and their image position were slightly shifted according to their spectra (FIG. 19) into a combined intensity distribution on the camera's sensor (see FIG. 20 for the experimental dispersion curve converting emission wavelength to pixel displacement on the camera sensor). This results in a spectral image where the combinations of fluorophore colors are converted to unique dual-spot point-spread-function (PSF) with their inter-spot distance indicative of their color combination (FIG. 11C bottom). CoCoS allows to symmetrically rotate the prisms along the optical axis of the fluorescence emission path, offering easy control over the spectral dispersion introduced to the image (see ref.³⁵). Therefore, the spectral resolution is optimized for each panel of reporter probes according to its fluorophore-pairs and multiplexing needs, to establish the best throughput and signal to noise ratio (SNR). Optimizing the spectral dispersion enables multiplexed registration of single molecule miRs.

Using CoCoS, all targets are imaged simultaneously in a single frame acquisition per field-of-view (FOV, about 130×130 μm²), reducing sample acquisition time by a factor of the number of fluorophores used, and eliminating cross-color photobleaching by consecutive excitations. Spectral registration allows expanding the palette of fluorophore options available for reporter tagging and also eliminates cross-talk between color channels and the need for color channels alignment and registration. In miRACLE, each FOV contains hundreds of miRs, each detected as a unique two-spots diffraction-limited PSF corresponding to the spectral emission signature of its fluorophore-pair (FIG. 11C). The unique inter-spot distances and their intensity distribution enable the classification of spectral PSFs and consequently facilitate visual differentiation between the target miRs. To guarantee miRACLE's ability to multiplex and classify different miR targets, a PSF simulator was designed using the Matlab® software (FIG. 11C bottom). The simulator input is composed of the fluorophores' and filter's spectra together with the induced spectral dispersion by CoCoS (FIGS. 19 and 20). It then simulates the spectral PSFs with an option of adding Gaussian and Poisson noises which provides a more realistic assessment of our probe classification capability.

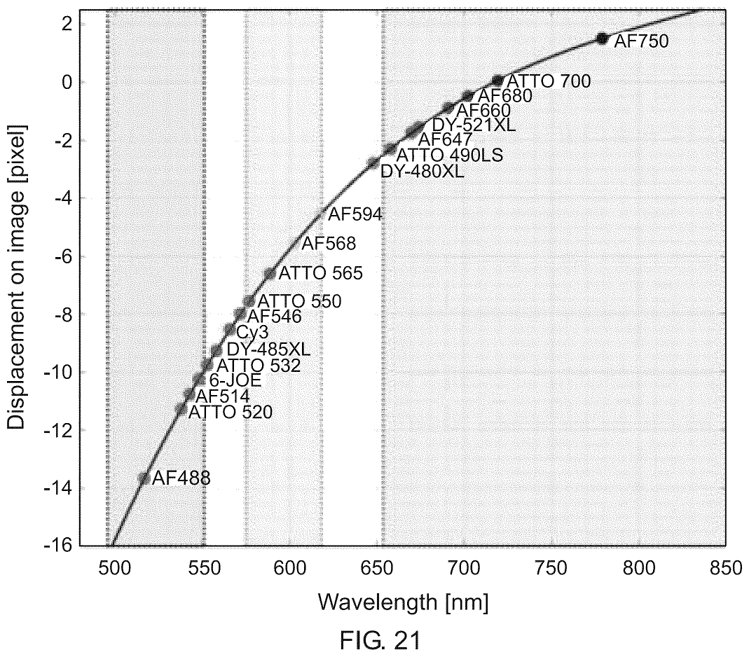

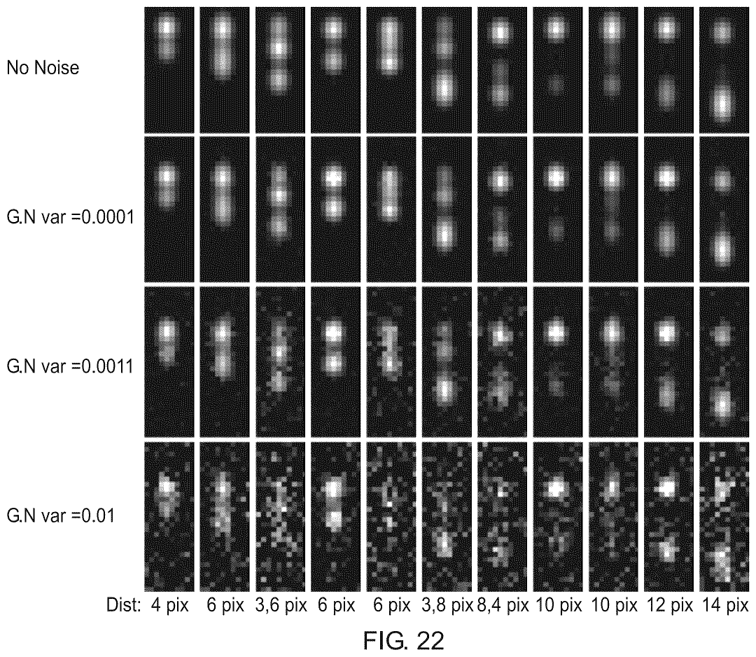

The PSF simulator allowed inputting various dual-fluorophores combinations and examine their induced spectral PSF on the system. This tool allowed to visually inspect the outcome of different fluorophore combinations (FIG. 21), and assess the number of spots and distances in their induced spectral PSFs' (selection of fluorophores which emit in multiple spectral windows of the multi-band filter, can create three and four spots intensity distributions as is shown in FIG. 22). The simulator can discover distinguishable fluorophore-pair combinations for maximal multiplexing capability. Choosing fluorophore pairs that give unique distances between PSFs' spots and visually distinguishable intensity distributions, allows to maximize the number of probes that can be simultaneously classified in a single sample. With a crude visual inspection of the simulator results of hundreds of fluorophore combinations (FIG. 21), the Inventors were able to assemble 11 fluorophore-pairs combinations that have distinguishable PSFs and inter-spot distances. These fluorophore pairs could potentially allow to simultaneously classify up to 11 miR targets in a single snapshot, with the same spectral resolution and with realistic SNRs (FIG. 22 and Table 7, below). On the other hand, the PSF simulator also allows one to find the optimal experimental setting for a specific experiment with a set probe panel, optimizing the inherent trade-off between spectral-resolution, SNR, and the maximal miR density. By visually inspecting the output PSFs the users can find the optimal spectral resolution and fluorophore-pair combinations according to the experimentally required number of miR targets.

As shown in FIG. 11C, the PSF simulator results closely resemble the experimental PSF extracted from three different experiments, each imaging a single miR target at a time. The simulated and experimentally calculated inter-spot distances are in exact agreement, although the intensity distribution between spots differs slightly due to axial focal point chromatic aberrations that were unaccounted for in the simulations.

After image acquisition, the miRACLE pipeline proceeds with automatic analysis of the entire image dataset analysis with the aim of detecting, identifying, and quantifying the single-molecule distributions of miR targets.

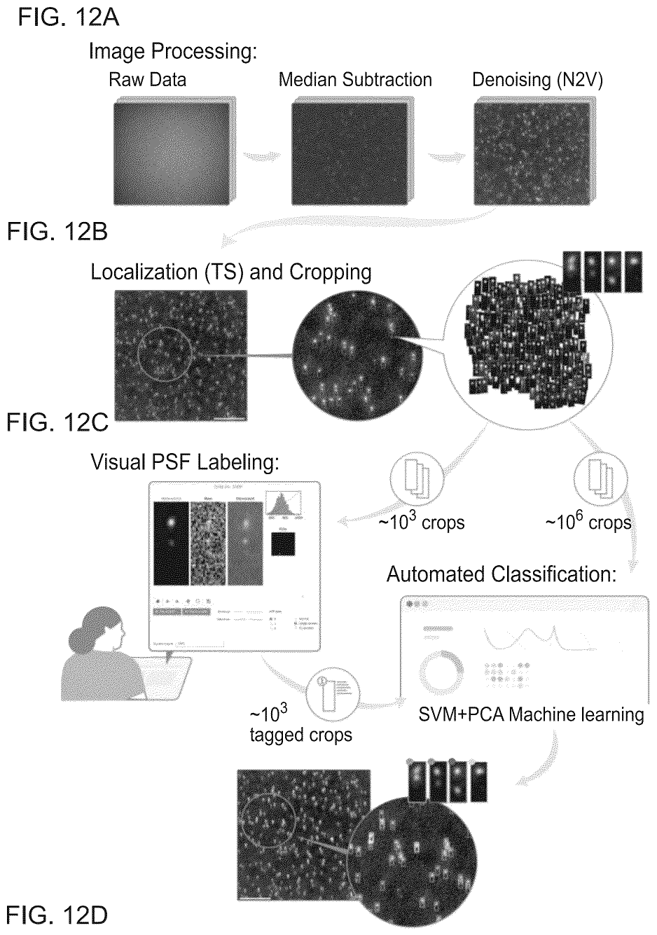

First, the raw image stacks are preprocessed to enhance the SNR for subsequent single-molecule detection and classification (FIG. 12A). The preprocessing protocol includes a standard background subtraction method utilizing pixel-median calculation across the entire multi-FOV stack to eliminate spatial background dependencies. Subsequently, a deep learning-based technique known as Noise2Void (N2V)³⁸ correction is applied (see methods and Table 8, below for details), which effectively eliminates local zero-mean noise and further improves the quality of the single-molecule PSFs. This preprocessing workflow ensures optimal data quality and enhances the accuracy of subsequent analysis and interpretation.

Next, to extract the relevant point spread function (PSF) data from the full FOV images, a cropping process is employed. This involves the localization of all two-dimensional diffraction-limited Gaussian shapes within the images using the ImageJ's ThunderSTORM (TS) plugin³⁹ (see methods and Table 9, below, for details). Subsequently, a rectangular region measuring 24×10 pixels is cropped around each localization (FIG. 12B). To ensure optimal performance without compromising recall, the TS localization threshold is set as low as possible while excluding single-pixel localizations. Since the spectral PSFs comprise two diffraction-limited Gaussian spots, the TS plugin detects each probe twice and crops rectangles around both the lower and upper spots. For downstream analysis, the upper crop is sufficient as it encompasses the complete double-spot PSF (FIG. 12B, rightmost crop examples). Consequently, it 50% of the generated crops may be classified as non-miR “noise”.

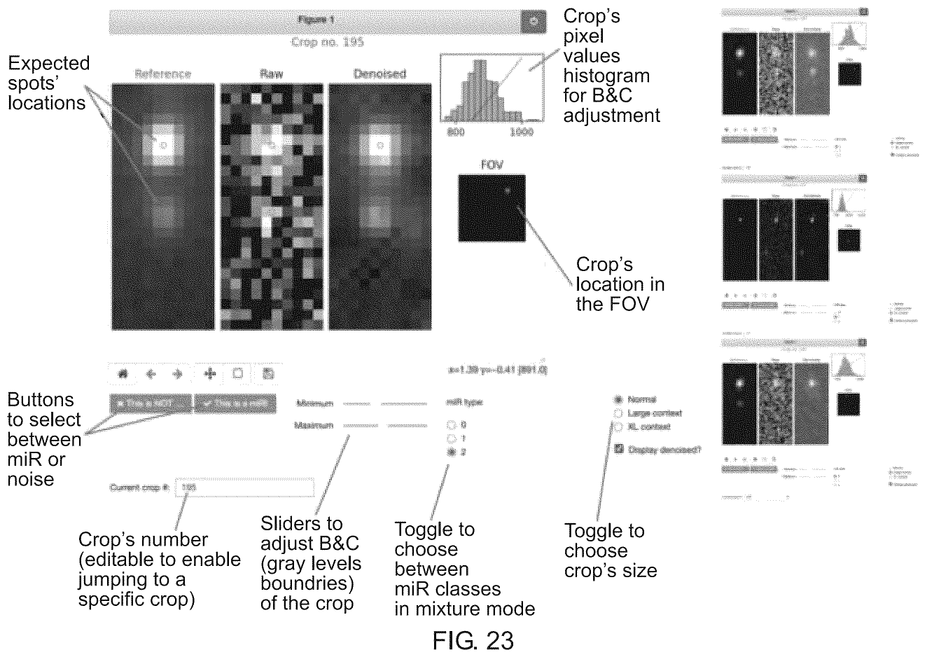

The crop classification and miR target expression profile reconstruction are performed using an automated pipeline based on principal component analysis (PCA) and support vector machine (SVM) machine-learning. The PCA-SVM model takes crops as input and assigns them to one of three miR types, or categorizes them as noise when they do not exhibit a strong correspondence with any of the miR probes (see methods and Table 10, below, for details). To train the model, small subsets containing about 1500 crops from each of the three single miR species datasets were manually curated using V-TIMDER (Visual Tagging Interface for Machine-learning DEcision Refining), a custom Graphical User Interface (GUI) tool for PSF labeling. V-TIMDER enables users to visually label the crops as either “noise” or the relevant miR PSF, generating a labeled dataset for the supervised machine-learning classifier. It provides various features that facilitate the users' PSF classification, such as plotting the expected PSF image and overlaying the expected spots' locations on the crop for easy visual comparison (FIG. 12C top left, detailed interface description in FIG. 23). The labeled results from V-TIMDER serve as training data for the automated PCA-SVM classifier, capable of classifying millions of crops from diverse miR distributions and mixtures (FIG. 12C bottom).

To validate the results of the automated classifier on mixed samples, V-TIMDER was further adjusted to allow visual classification of such samples (FIG. 23). In this “mixture” mode, rather than the binary choice between miR or noise for each crop as in the single-species case, the user has the flexibility to assign each crop to one of the three PSF types or classify it as noise.

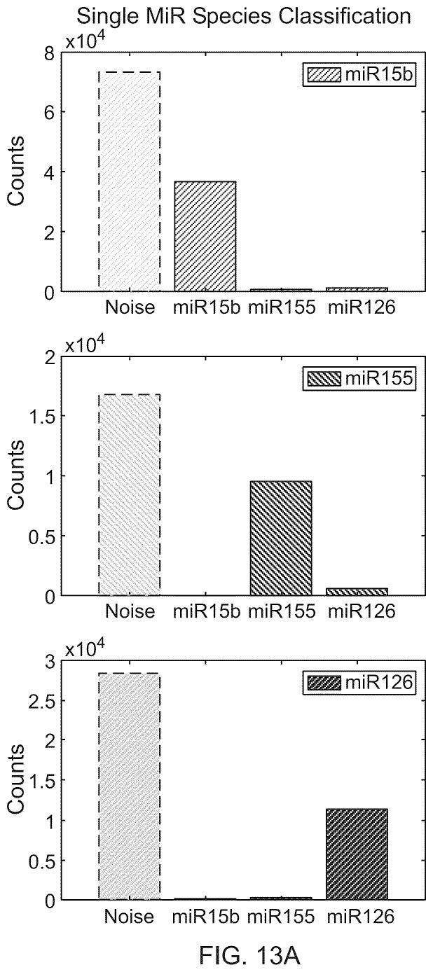

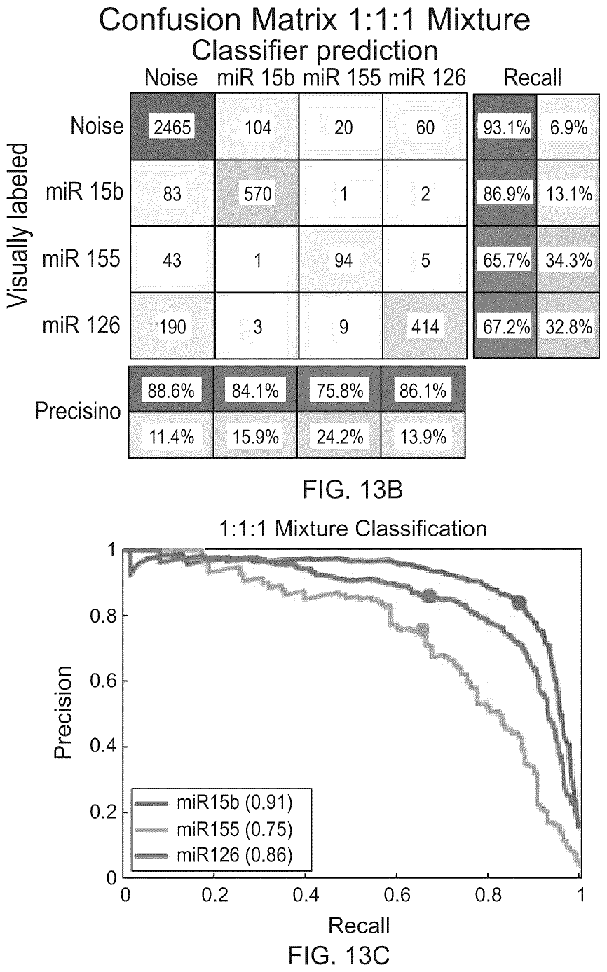

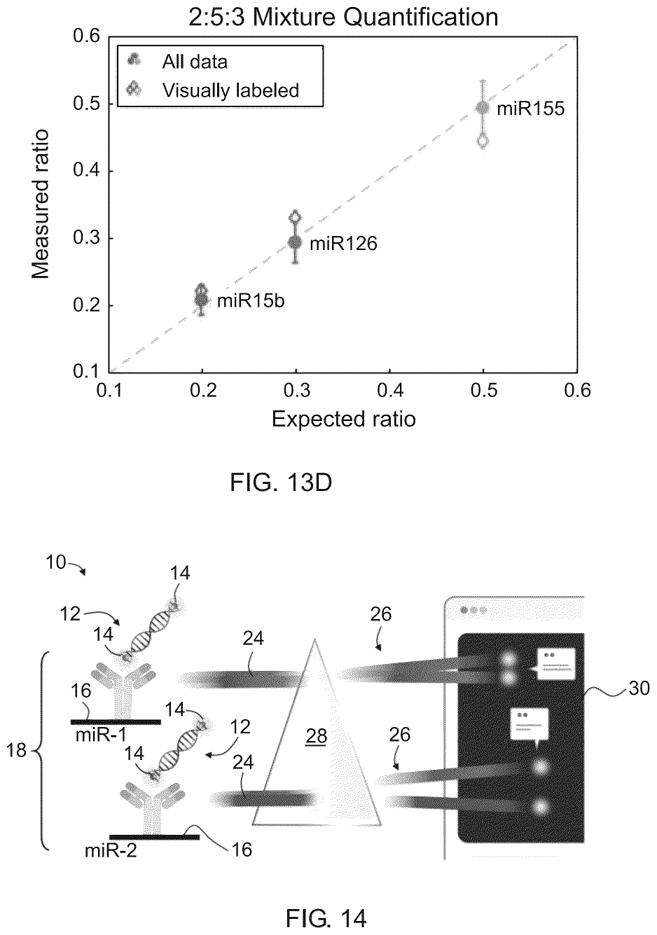

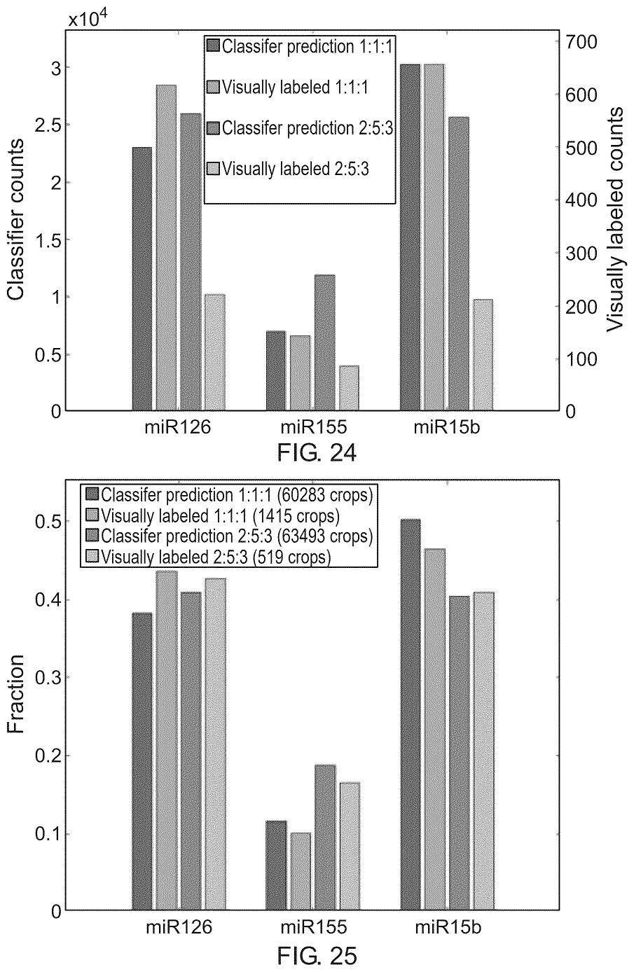

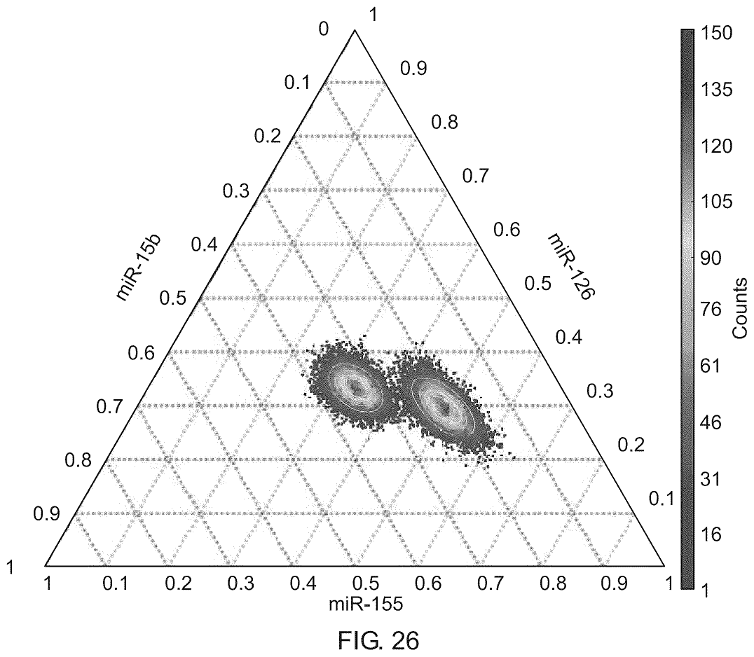

The classified results are compiled to obtain the miR count distributions, which can be utilized for downstream analysis and future diagnostic applications. To benchmark the classifier performance, the three different samples of a single miR-species were analyzed individually, demonstrating the accurate classification capability of the automated classifier with minimal confusion between miR types (FIG. 13A). Subsequently, the Inventors investigated two mixtures of the three miRs with ratios of 1:1:1 and 2:5:3 (miR-15b-5p, miR-155-5p, and miR-126-3p, respectively). To evaluate the classifier's recall and precision capabilities, V-TIMDER was utilized to visually assess a small subset of crops taken from the 1:1:1 mixture's dataset (4,064 visually labeled crops out of 197,963 classified crops). The labeled data was used as a test set to benchmark the performance of the automatic classifier on mixed samples. Comparison of visually labeled and classifier's results are summarized with the confusion matrix in FIG. 13B. The precision and recall for each miR type were evaluated from the matrix (marginals in FIG. 13B and circles in FIG. 13C). Evidently, the classifier predominately confounds between noise and the different classes, and hardly mixes between miR types, allowing the Inventors to evaluate the performance of the classifier for each miR independently by performing a binary classification (see methods) and obtaining precision-recall (PR) curves (FIG. 13C). The PR curves and their corresponding area under the curve (AUC-PR)⁴⁰ demonstrate a varying classification performance between miR targets, with the best performance for miR-15b-5p which has a more distinguished PSF. Essentially, the confusion matrix for the 1:1:1 mixture encompasses all the systematic classification errors. Thus, this equidistributed sample was use to correct the ratiometric readout for various experimental contributions such as different initial single-species concentrations (visible in FIG. 13A), competitive binding effects which are known to affect the measured ratios³¹, and minute PSF differences between the single-species and mixtures experiments due to experimental focal changes (FIGS. 13A, 13B, 24 and 25). Since these physical effects skew the count distributions ratio in a constant manner, the observed count distributions can be normalized to estimate the true miR targets ratios. By employing the inverted row-normalized confusion matrix on the classifier results and normalizing the result with the 1:1:1 absolute count distribution, the Inventors effectively corrected all inherent physical and classifier errors, obtaining a more precise representation of the miR mixtures' underlying ratios (see methods for details). This is demonstrated on a different mixture with 2:5:3 ratio retrieving a very close result of 2.10:4.95:2.95 ratio with our pipeline (FIG. 13D). The confusion matrix also allows for assessing the overall errors of our pipeline (see methods for details). Since the confusion matrix values are discrete measurements, it was assumed that they have a multinomial noise distribution around the observed values (depicted in FIG. 13B), allowing to generate multiple confusion matrices and determine the miR ratio's confidence bounds (FIG. 13D, error bars correspond to two standard deviations or 95% of possible results. Further details are given in the methods). The evaluated ratio errors correspond well to the ratio results of a V-TIMDER visual classification benchmark test on a small subset of crops taken from the full dataset (1,565 crops out of 214,231 crops in the 2:5:3 dataset, out of which 519 were visually assigned to one of the miR classes and the rest were classified as noise). From this analysis, the dominant uncertainty in estimating the ratio distribution arises from misclassifications of miRs 126 and 155, while miR 15b is better classified in our model (see FIG. 26 for the full distributions of simulated ratios). Nevertheless, according to this uncertainty estimation, in the worst-case scenario, it is expected that variations larger than 10% in mixture abundance ratios will be distinguished. Other error sources may also be considered according to some embodiments of the present invention.

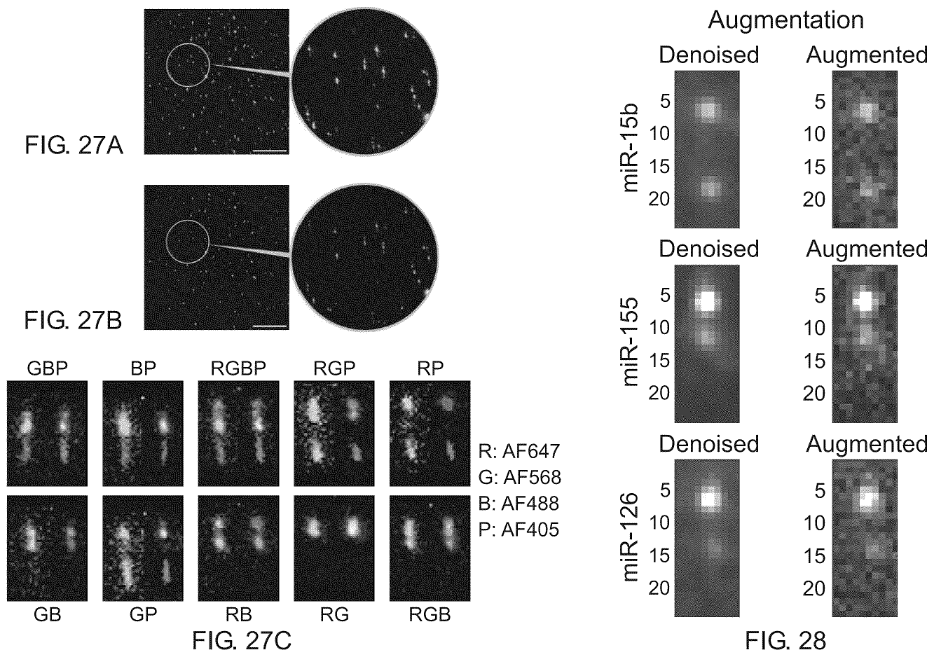

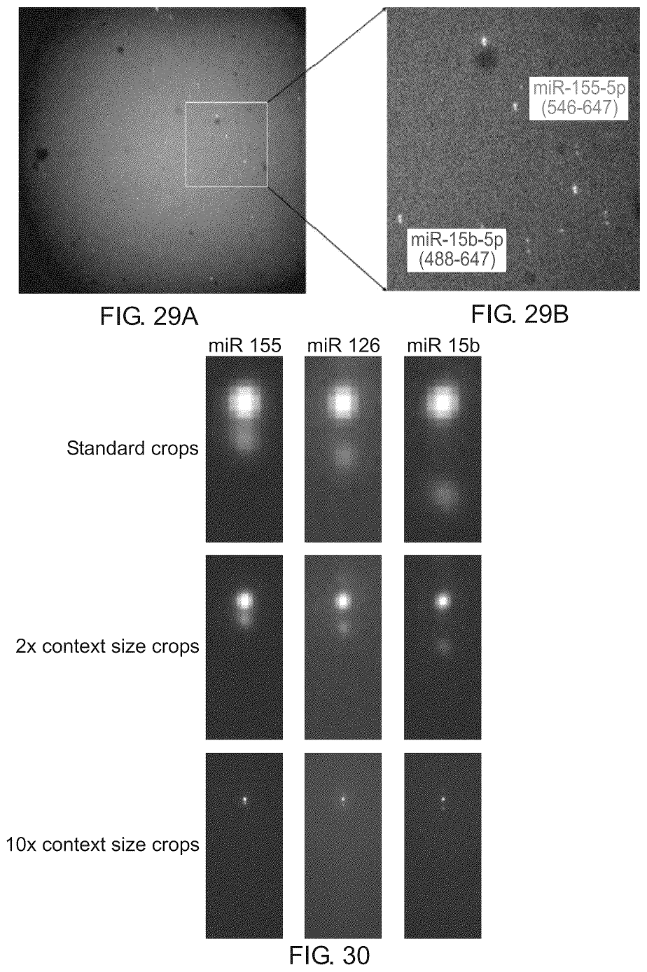

The results presented above demonstrate the capability of confidently detecting and reconstructing mixture distributions of three miR types simultaneously. The simulations show that the method can multiplex many types of miRs (FIG. 22). MiRACLE's multiplexing capabilities can be further enhanced by introducing more fluorophores probes (e.g., 3- and 4-fluorophores probes), allowing to combinatorically increase the uniquely resolvable spectral PSFs that can be classified by miRACLE (see FIG. 27 showcasing 10 unique PSFs with only four fluorophore combinations, demonstrated using 100 nm colored silica beads). MiRacle provides single-molecule detection capability of unamplified miR targets with ultimate sensitivity. The method is fast, sensitive, and extremely cost-effective (less than $4 per sample, Table 6), providing robust profiling of small clinical panels of native miR and other RNA targets (see FIG. 29 for a demonstration of multiplexed detection of two miR targets in small RNA extracted from human plasma). The V-TIMDER tool of the present embodiments can be implemented in a wider context of single-molecule applications involving PSF engineering⁴¹.

### Methods

### Slides Preparation

The miRs were captured on borosilicate glass coverslips (D 263 Schott glass, 75.5×25.5 mm², ibidi GmbH, Germany), passivated with poly-ethylene glycol (PEG). In brief, after cleaning, each coverslip was hydroxyl terminated with freshly prepared KOH. The coverslip was then pegylated with a mixture of Methoxy PEG Silane (Laysan Bio Inc. AL, USA) and mPEG-Silane-Biotin, MW5000 (Laysan Bio Inc. AL, USA) in a 1:100 stoichiometry in dehydrated HPLC grade Ethanol. A six-channel ibidi sticky-slide (μ-Slide VI 0.4, ibidi GmbH, Germany) was mounted on top of the pegylated coverslip. Each channel was hydrated with 100 μL of DNase I and RNase-free deionized water for 15 min and then equilibrated with 100 μL PBS for 30 min. Following this, each channel was activated with 100 μL of monoclonal Anti-DNA-RNA hybrid S9.6 antibody (S9.6, Mouse IgG2a kappa Isotype, conjugated with Streptavidin, Ab01137-2.0, Absolute Antibody, Oxford, UK) to capture our targeted biomarkers. Thus prepared channels were able to capture targeted miRs hybridized with labeled DNA probes as DNA:RNA duplex.

### Protocol for Glass Coverslips Cleaning and PEGylation

### Coverslips Cleaning

(i) Mark coverslips on the right top side using diamond cutter tool. Take care to avoid glass breaking.
(ii) Rinse staining jar twice with isopropanol followed by ddH₂O.
(iii) Place marked coverslips in staining jar and rinse twice with isopropanol followed by ddH₂O.
(iv) Fill the staining jar with 2% Hellmanex. Sonicate 30 min at 30° C. and discard the solution.
(v) Rinse the coverslips thoroughly with ddH₂O, repeat 8 times.
(vi) Sonicate the staining jar with ddH₂O for 10 minutes at 30° C., and discard.
(vii) Fill the staining jar with freshly prepared 4M KOH (28.05 g KOH in 125 ml ddH₂O) and sonicate the slides for 100 min.
(viii) Rinse the coverslips thoroughly with ddH₂O, repeat 8 times to remove traces of KOH.
(ix) Feel the staining jar with technical grade (96%) ethanol, and discard.
(x) Rinse the coverslips thoroughly with ddH₂O.
(xi) Blow dry the coverslips with nitrogen.
(xii) Bake the coverslips for 3 hours at 500° C. (can be left overnight).
(xiii) Rinse staining jar twice with ethanol followed by ddH₂O.
(xiv) Place the coverslips in the cleaned staining jar, fill it with 1M KOH (7.01 g in 125 ml ddH₂O) and sonicate 30 minutes at 30° C.
(xv) Rinse the coverslips thoroughly with ddH₂O, repeat 8 times to remove all traces of KOH.

### Coverslips PEGylation

Since the lifetime of the PEG solution is short, it was prepared immediately before its application on coverslips.
(i) Prepare PEG solution (3% by volume) in dehydrated high-grade ethanol (HPLC grade). For 500 μL of a 1:100 PEG-Biotin/mPEG Silane (for 4 coverslips):
   (a) Dissolve 15 mg Methoxy PEG-Silane (mPEG-Silane MW5000, Laysan Bio Inc. AL, USA) and 0.15 mg Biotin-PEG-Silane (Biotin-PEG-Silane MW5000, Laysan Bio Inc. AL, USA) in 475 μL dehydrated ethanol. Mix by warming the solution for 1 minute to 35° C. followed by pipetting
   (b) Add 25 μL glacial acetic acid (5% V/V) just before applying onto the coverslips.
(ii) Degas the solution: centrifuge for 1 min at 16,000 g to remove air bubbles.
(iii) Blow dry the pairs of cleaned coverslips with nitrogen and place in an empty pipette tips box partially filled with ddH₂O.
(iv) For each pair of coverslips, sandwich 250 μL of the PEG solution between the coverslips (the engraved side facing to the center), avoid creating bubbles.
(v) Cover box with tin foil and incubate overnight in a dark place at room temperature.
(vi) Carefully separate the coverslip pairs, take care not to break the glass.
(vii) Rinse carefully with ethanol followed by ddH₂O and thoroughly blow dry with nitrogen.
(viii) Store each coverslip separately in a 50 ml falcon filled with nitrogen. Seal the cap with parafilm and store in −20° C.

### Reporter Probe Sequences

Capture probe for hsa-miR-15b-5p
(ATTO488-ATTO647N):
SEQ ID NO: 1
/5ATTO488K/TT AGT TGT AAA CCA TGA TGT GCT
GCT AAT GTA/3ATTO647NN/
Capture probe for hsa-miR-155-5p
(AF546-ATTO647N):
SEQ ID NO: 2
/5Alex546N/TT AGT AAC CCC TAT CAC GAT TAG
CAT TAA ATG TA/3ATTO647NK/
Capture probe for hsa-miR-126-3p
(ATTO488-ATTO565N):
SEQ ID NO: 3
/5ATTO488N/AC TTA GTC GCA TTA TTA CTC ACG
GTA CGA ATG TAT C/3ATTO565N/

### Synthetic miR Experiments

In all experiments with synthetic microRNAs (miRs) and their complementary single-stranded DNA capture probes (ssDNA), 100 μL of the duplex at 50 pM concentration was used in each channel which was preincubated with S9.6 antibody. After hybridization, a total of 31 μL miR mixture was applied on the immobilized S9.6 antibody in one channel, incubated for 45 min, and washed 3× with PBS before imaging.

### Human Plasma Experiments

All small RNAs (including miRs) were purified from 500 μL plasma using miRNeasy Serum/Plasma Advanced Kit (QIAGEN GmbH, Hilden, Germany) in 15 L of Rnase free water, and stored in −20° C. 15 L PBS was added to the purified RNA extract, following 0.1 fmol (1 L of 5 pM) of miR-155-5p and miR-15b-5p capture probes (FIG. 11C). The solution was left for 3 hours hybridization at room temperature. After hybridization, a total of 31 μL of the solution was applied onto the pre-immobilized S9.6 antibody slide, incubated for 45 min, and washed 3 times with PBS before imaging (results shown in FIG. 29).

### Multi-Color Silica Beads Experiment

For each combination of fluorescent colours presented in FIG. 27 a 5 L of 100 nm silica beads (SB) functionalized with azide (Si100-AZ-1, Nanocs, NY, USA) were added to 100 L of 1:1 ddH2O:Ethanol solution and vortexed thoroughly. The Inventors then added to the solutions 0.2 μL of 10 mM AF-405, AF-488, AF-568, AF-647 DBCO conjugated dyes (AF647-DBCO, AF488-DBCO, AF405-DBCO, Jena Bioscience, Germany; AFDye 568-DBCO, Click Chemistry Tools, USA) according to the required combination of colors, followed by vigorous pipetting and vortex for creating homogenised distribution. The solution was left to incubate 3 hours at 37° C. after which the beads were cleaned from residual free fluorophores by the following steps:
(i) Centrifuge at 17,000 RPM at 4° C. for 15-30 min (or until a pellet is visible at the bottom of the tube. The pellet should be coloured according to the dye used).
(ii) Gently remove the liquid while avoiding disturbing the pellet.
(iii) Add 100 μL of 1:1 ddH2O:Ethanol, pipette vigorously and vortex for homogeneous distribution.
(iv) Repeat (i)-(iii) four times. At the last repeat avoid (iii) and instead proceed to (v).
(v) Suspend the washed pellet in 30 μL of ddH2O to obtain stock coloured silica beads.

The stock solution was diluted 1:100 in ddH2O before imaging.

### Optical Setup

Excitation: For excitation, the Inventors used three lasers (Cobolt AB, Sweden) with wavelengths 488 nm (MLD 488, 200 mW max power), 561 nm (Jive 561, 500 mW max power), 638 nm (MLD 638, 140 mW max power). All lasers were mounted on an in-house designed heatsink which coarse aligned their beam heights. Each laser beam was passed through a clean-up filter (LL01-488-12.5, LL01-561-12.5, LL01-638-12.5, Semrock, USA) and expanded to 12.5-20× its original diameter (3×LB1157-A, 3×LB1437-A, Thorlabs, USA). A motorized shutter (SH05, Thorlabs, USA) was used for modulating on/off the solid-state 561 nm laser, while the diode lasers were modulated directly on the laser head. The beams were then combined into a single beam using long-pass filters (Di03-R488-t1-25.4D, Di03-R561-t1-25.4D, Semrock, USA). To homogenize the excitation profile of the sample, the combined beam was passed through an identical setup to the one described in the work of Douglass et al.⁴². In short, the combined beam was injected into a compressing telescope (AC254-150-A-ML, AC254-050-A-ML, Thorlabs, USA) with a rotating diffuser (24-00066, Süss MicroOptics SA, Switzerland) placed about 5 mm before the shared focal points of the telescope lenses. A series of 6 silver mirrors (PF10-03-P01, Thorlabs, USA) was then used to align the beam into a modified microscope frame (IX81, Olympus, Japan), through two identical microlens arrays (2×MLA, 18-00201, Süss MicroOptics SA, Switzerland) separated by a distance equal to the microlenses focal length and placed inside the microscope frame. The homogenized beam was reflected onto the objective lens (UPlanXApo 60×NA1.42, Olympus, Japan) by a four-band-multichroic mirror (Di03-R405/488/532/635, Semrock, USA). The sample was placed on top of motorized XYZ stage (MS-2000, ASI, USA) with an 890 nm light-emitting diode (LED) based autofocus system (CRISP, ASI, USA), which enabled scanning through multiple fields of view.

Emission: The emitted fluorescence light was gathered by the same objective and transmitted through the multichroic mirror onto a standard Olympus tube lens to create an intermediate image at the exit of the microscope frame. This image was passed through a multi-band emission filter (FF01-440/521/607/694/809-25, Semrock, USA) and was then directed into a magnifying telescope (Apo-Rodagon-N 105 mm, Qioptiq GmbH, Germany and Olympus' wide field tube lens with 180 mm focal length, #36-401, Edmund Optics, USA), with two commercial direct vision prisms (117240, Equascience, France) placed within the infinity space between the lenses and mounted on two motorized rotators (8MR190-2-28, Altechna UAB, Lithuania) controlling the prisms' angles around the optical axis. The final image was acquired on a back illuminated sCMOS camera (Prime BSI, Teledyne Photometrics, USA).

Image acquisition was coordinated using the micro-manager software⁴³, controlling camera acquisition, laser excitation, XY stage location, and prism rotator angles. The camera and laser excitation were synchronized using a TTL controller based on an Arduino® Uno board (Arduino AG, Italy).

### Image Acquisition

The sample lanes were scanned laterally and imaged with a single acquisition per FOV, obtaining approximately about 2000 FOVs per sample. The different fluorophores used in our probes design have different photophysical properties effecting their overall brightness. Therefore, since all lasers excite the probes simultaneously, individual lasers intensities were adjusted to achieve homogeneous intensity profiles of the probes' PSFs.

For imaging a single exposure per FOV with a relatively long exposure time (800 ms per frame) compared to standard multi-frame fluorescence imaging (30-100 ms per frame) were used. This long exposure did not contribute to extensive photobleaching as excitation power was distributed over a large field of illumination (130×130 μm²) resulting in relatively low irradiation at the sample (about 0.2 kW/cm²). Furthermore, even if a fluorophore did bleach during the single-frame acquisition, its signal was still recorded during this exposure resulting in optimized probe detection and SNR.

The same optimization was done in the colored silica beads experiment, using lower laser powers to excite the higher fluorophore densities found on the beads. The acquisition parameters are described in Table 5.

TABLE 5
Image acquisition parameters

| Experimental | | | Emission | Exposure | Laser Intensities at |
|---|---|---|---|---|---|
| target | Excitation | RPA | Filter | time | laser output |
| Synthetic | All lasers | 177.5 | FF01-440/ | 800 ms | 638 Laser: 90 mW |
| miRs | simultaneously | | 521/607/ | | 561 Laser: 90 mW |
| | | | 694/809-25 | | 488 Laser: 160 mW |
| Colored 100 | All lasers | 178 | NF03-405/ | 250 ms | 638 Laser: 30 mW |
| nm silica | simultaneously | | 488/561/ | | 561 Laser: 10 mW |
| beads | | | 635E-25 | | 488 Laser: 40 mW |
| | | | | | 405 Laser: 60 mW |
| | Sequential | 178 | NF03-405/ | 250 ms | 638 Laser: 30 mW |
| | laser excitation | | 488/561/ | | 561 Laser: 10 mW |
| | | | 635E-25 | | 488 Laser: 40 mW |
| | | | | | 405 Laser: 60 mW |

### Image Processing

The image processing scheme used for generating miR distributions from the raw dispersed images, was divided into four sub-processes: i) image pre-processing, ii) PSF detection, iii) PSF visual labeling with V-TIMDER for training the automatic classifier, iv) automatic PSF classification.

### Image Pre-Processing

Before analyzing the miRs, the images were processed in the following way in order to improve the SNR:
(i) First, to remove the inhomogeneous background in each FOV was removed by subtracting a pixel-wise median calculated across all FOVs in the experiment.
(ii) Outlying FOVs were pruned based on a rough statistical measure: keep only the FOVs whose mean of the (median-subtracted) positive pixels and the mean of the negative pixels are smaller in absolute value than some threshold. This filters out FOVs with extreme features such as bubbles, and FOVs with a background that did not agree with the median. The threshold is chosen so about 80% of the FOVs are kept.
(iii) Noise2Void (N2V), which is a self-supervised deep learning algorithm, was then used to denoise the images and improve the SNR. The main assumption of the algorithm is that the noise is pixel-wise independent, an assumption that holds for the pruned, median-subtracted images. The hyperparameters for the N2V model are detailed in the supplementary material (Table 8).

### PSF Detection

FIJI's ThunderSTORM plugin³⁹ was used to find blobs (peaks) in the denoised images. This provides the x,y coordinates of each blob in each FOV. The thresholds were selected such that all the top blobs of all miRs in the image were detected, in addition to “noise blobs” which are blobs that are not part of a miR, or the bottom blob of a miR. Threshold selection was done based on thorough visual inspection of a few randomly selected FOVs.

Blobs that are very close to each other in the same FOV were merged into a single blob at their mean position. The distance threshold below which blobs are merged is significantly smaller than the distance between miRs. Blobs that are very close to the FOV boundary were discarded.

Rectangular crops around each blob were taken in both the noisy (median-subtracted) and denoised images. The dimensions of the crops, 24×10 pixels, were selected such that both top and bottom blobs appear in each miR crop. The crop dimensions were selected such that each crop will contain the complete point-spread-function (PSF) information of the three miR targets' spectral signatures. Therefore, the top blob of each TS detected PSF was centered around the sixth pixel from the top of the crop and at the 5th pixel from the left (centered), leaving 18 pixels below to encapsulate the bottom blob (maximal anticipated peaks distance of 11 pixels, see FIG. 1C in the main text). The empirical standard deviation of the blobs was determined as about 1.6 pixels, therefore the chosen dimensions of the crops allow it to contain the complete information of a single PSF, while minimizing the chance for detection of multiple PSFs within the same crop.

### Visual PSF Labeling Using V-TIMDER

To empirically estimate each miR's PSF from the data (FIG. 30) the pixel-wise median of the single miR species were calculated over about 10⁵ noisy crops. These empirical PSFs have an excellent SNR with two distinct blobs.

To generate training, validation and test datasets for our machine-learning model, a few thousands of blobs taken from a small (about 5-10) number of FOVs were randomly chosen. For these blobs, in addition to the standard crops, larger crops (×2 and ×10 the standard crop size, see FIGS. 23 and 30) from both the noisy and denoised images are taken to provide a better visual context of the blobs. This ensemble of crops, as well as the empirical PSFs, were fed to V-TIMDER, a custom-built GUI that allows convenient visual classification of the crops. This GUI has two modes: a “binary” mode where the user needs to determine for each blob whether it is the top blob of a miR or not, and a “mixture” mode where the user also determines which miR species it belongs to. The binary mode is used for the single-species datasets, and the mixture mode is used for the mixture datasets. In this Example, 1,701 (miR 15b), 1,783 (miR 155) and 1,795 (miR 126) crops we visually classified from the single-species datasets, and another 4,064 and 1,565 crops were classified from the 1:1:1 and 2:5:3 mixtures, respectively.

### Automatic PSF Classification Using Machine Learning

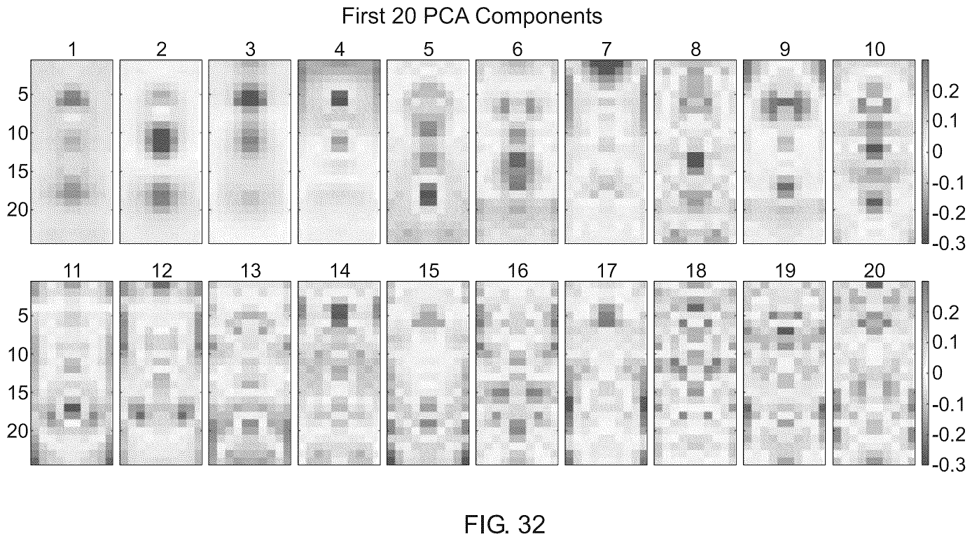

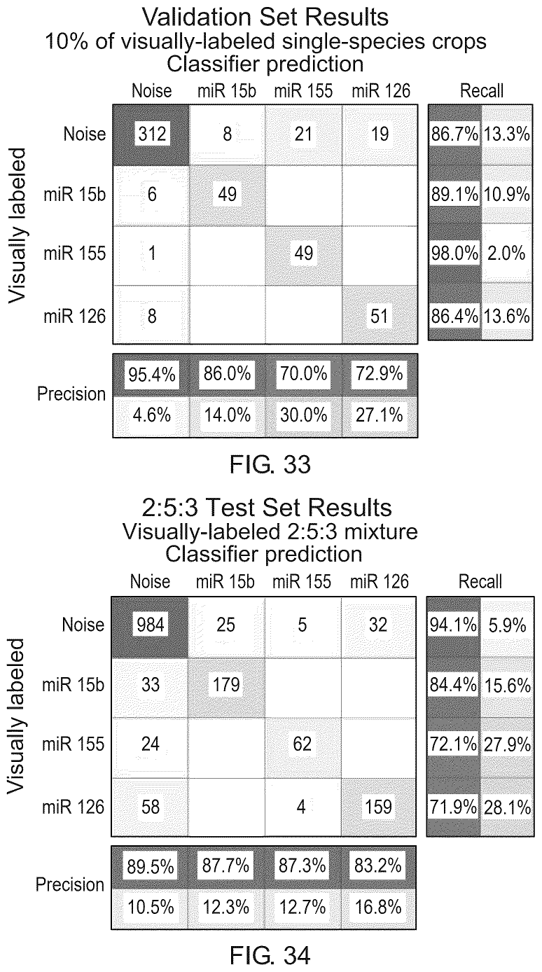

The full classifier's pipeline according to some embodiments of the present invention is provided as a flowchart in FIG. 31.
1. Classifier training and validation: For the training process we used 90% of the denoised crops from the single-species visually-labeled dataset. The remaining 10% were used for model validation, obtaining metrics of classifier performance and hyperparameter tuning. The training was carried out for both classifier's modules with preceding data augmentation and preprocessing steps:
   a. Data augmentation: The training dataset was augmented by adding multiple realizations of a weak random pixel-wise Gaussian noise to each labeled denoised crop (see FIG. 28, for example, augmentations and Table 10 for details). We found this step to be crucial for the classifier's success. This also increased our training dataset size by a factor of three.
   b. Crops preprocessing: All crops were preprocessed by the following pipeline:
      i. Background subtraction: For each denoised crop, subtract the median of the pixels at the crop edges. If this causes any of the four central pixels of the top blob to become negative, discard the crop. Otherwise, replace negative pixels with zero values.
      ii. Normalization: Normalize each crop by a factor k such that the four central pixels of the top blob have a mean of 1 (see Table 10 for details). The normalization constant k is saved for future use (item d below).
      iii. Symmetrization: add the horizontally-flipped mirror image of the crop to itself. This produces a symmetrical crop. We keep only the right half, reducing the crop size from 240 pixels to 120.
   c. Unsupervised PCA training: The preprocessed symmetrized crops from the single-species visually-labeled dataset were fed into an unsupervised PCA training procedure, extracting the 20 most significant components characterizing the dataset to be later used for dimensionality reduction (see FIG. 32 for learnt PCA components).
   d. Preprocessing before SVM Classification: The symmetrized crops together with their saved k factor were further processed before being fed to the SVM classifier:
      i. Dimensionality reduction: Project the symmetrized crop on the learnt 20 PCA components to obtain 20 coefficients.
      ii. Standardization: Normalize the 20 coefficients as well as k to have a zero mean and a unit variance.
   e. SVM training: The visually-labeled single-species datasets' standardized PCA coefficients, k values and labels were fed to a RBF-kernel SVM classifier^{44,45} (see Table 10 for classifier's details). The classifier chooses between four classes, representing the three miR species and a noise class for crops that do not contain a miR (either the bottom spot in a miR or a false detection from ThunderSTORM). The resulting SVM model was saved for further use.
   f. SVM classification: For the validation and test data, the learnt SVM model was directly applied on the standardized PCA coefficients and k values to provide classification. The final classification by the classifier can be carried out in two ways:
      i. Classification using Scikit-Learn's predict method⁴⁶, which returns the predicted class. This method was used for all practical purposes including training, validation, testing and classifying (displayed in FIGS. 13A, 13B, 13D, 33 and 34).
      ii. Probability prediction using scikit-learn's predict_proba method⁴⁷, which returns a probability vector of length four, describing the model's estimation of the probability that the crop belongs to each of the classes. This method was used to generate the PR curves (FIG. 13C).
   g. Validation: the performance of the trained model was validated on 10% of the labeled dataset (see confusion matrix in FIG. 33). The validation crops processing was performed according to steps b and d.
2. Deployment: The 1:1:1 and the 2:5:3 mixture datasets, as well as the unlabeled subsets of the three single-species datasets, were fed through the above pipeline (steps 1b, 1d, 1f) for the purposes of calibration and testing, as detailed below.

### Precision and Recall

The labeled subset of the 1:1:1 mixture was uses both for evaluating the model's performance (with respect to the V-TIMDER true labels) and for calibrating the classifier. Using the classifier on this subset, the Inventors obtain a 4×4 confusion matrix Cᵢⱼ (FIG. 13B) whose entries are the number of blobs whose true label (according to V-TIMDER) is i and were predicted to be in class j. The matrix C is used both for performance evaluation and model calibration.

The recall for class i, rᵢ is the fraction of the true occurrences of this class that were correctly detected, rᵢ=Cᵢᵢ/Σⱼ Cᵢⱼ. Similarly, the precision pᵢ the fraction of blobs classified as class i, whose true class is indeed i, pⱼ=Cⱼⱼ/Σᵢ Cᵢⱼ. The circle markers in FIG. 13C are the precision and recall for each miR class.

A more descriptive metric of the classification strength is the precision-recall (PR) curves for the 1:1:1 subset, shown in FIG. 13C. These curves are generated by using the classifier's probability prediction method, in which the model outputs a probability vector Pᵢ, corresponding to the predicted probability that the sample belongs to class i. For each of the three miR classes a binary classification for that miR class was performed by thresholding Pᵢ. The threshold is varied between 0 and 1, and for each threshold we calculate the binary classification's precision and recall.

### Calibration and Testing

For each dataset the classifier returns a vector of length 4, which is denoted by h, corresponding to the predicted counts of occurrences in each class. The relation between h, and the true counts of these classes, h′, is given by definition, by h=Ĉ h′, where Ĉ is the row-normalized confusion matrix Ĉᵢⱼ=Cᵢⱼ/Σₖ Cₖⱼ. Therefore, to obtain the best estimate of true counts, the predicted class counts h was multiplied by the inverse of Ĉ. This gives the final estimation of the abundance of each class.

Doing this procedure for each of the unlabeled 1:1:1 and 2:5:3 mixture datasets, the Inventors obtain the estimation for the counts, h′₁₁₁ and h′₂₅₃. The element-wise division h′₂₅₃/h′₁₁₁ gives the estimate for the miR ratios in the 2:5:3 mixed dataset. This division accounts for experimental effects as mentioned in the text such as competitive binding of different miR types and PSF variations. This estimate, excluding its noise class, was normalized so its three entries sum to 1, as is illustrated in FIG. 13D.

### Uncertainty Estimation

To estimate our prediction uncertainty, the above procedure was repeated but with noise added to the confusion matrix C and counts vectors h₁₁₁ and h₂₅₃. Specifically, C was replaced with a pair of matrices drawn from a multinomial distribution whose mean value is C, and the counts h₁₁₁ and h₂₅₃ were replaced with counts drawn from Gaussian distributions with means equal to h₁₁₁ and h₂₅₃ and standard deviations of 5% of the means. Each matrix from the pair of randomized matrices was inverted to “unconfuse” the randomized count vectors, and the resulting h′₁₁₁ and h′₂₅₃ were divided and normalized to obtain a miR ratio vector. This randomization procedure was repeated 10,000 times to obtain an ensemble of ratio vectors (see FIG. 26). The inventors then fit a 2D Gaussian to this ensemble to obtain the mean and covariance matrix. The ratios in FIG. 13D correspond to the mean vector projected onto the single-species vectors on the simplex, and the errors correspond to a 95% confidence interval (two standard deviations) for each miR type, calculated by projecting the covariance matrix onto the single-species vectors.

### PSFs Simulations

All simulations were performed by a computer code programed using the Matlab® software package. A short description of the pipeline is provided below:
(i) Excitation and emission spectra of 16 commercial fluorophores together with our four-notch filter were downloaded from Semrock's SearchLight spectra viewer (names of fluorophores are provided in the supplementary Table 7). Each of the fluorophore's spectrum was multiplied by the filter's spectrum to produce the actual spectrum visible on our camera.
(ii) The wavelength to pixels displacement calibration curve of our CoCoS setup (which was calculated previously³⁵ was adjusted according to the experimentally used relative prism angle (RPA) by multiplying the entire curve by sin((180-RPA)/2).
(iii) Chosen double-fluorophore combinations were then simulated by converting each fluorophore's spectrum into a diffraction-limited dispersed image. This was done by assigning a Gaussian with unity amplitude and 1.15 pixel standard deviation to each wavelength in the emission spectrum. Each Gaussian was displaced according to the RPA-adjusted displacement curve and summed together with other Gaussians. Finally, the total summed intensity of all Gaussians was normalized to unity and multiplied by an excitation efficiency factor which was calculated by the excitation spectrum value (fractions only) at the excitation laser wavelength.
(iv) This process was repeated for the second fluorophore and both images were summed to provide the dual-fluorophore spectral image.
(v) When needed, a noise model was added to the simulated dual-fluorophore spectral PSF image using the “imnoise” function in Matlab. The noise model used in this work was a sum of a Poisson distributed shot-noise and Gaussian noise with a constant mean of 0.3 and a changing variance (see FIG. 22).

TABLE 6
Cost of goods analysis

| | | | | Total | Cost/ |
|---|---|---|---|---|---|
| | | Cost | Amount/ | No. of | Sample |
| Material | Amount | ($) | Sample | Samples | ($) |
| Mirneasy | 50 preps | $750 | 1 prep | 50 | $15.00 |
| plasma | | | | | |
| advanced | | | | | |
| kit- | | | | | |
| Qiagen | | | | | |
| 5 Reporter | 0.25 nMol | $9,750 | 0.1 fMol/ | 2500000 | $0.0039 |
| probes | Guaranteed | | Sample | | |
| S9.6 | 100 ul, | $498 | 60 pM * | 167,000 | $0.0030 |
| Antibody | 6.8 uM | | 60 uL | | |
| (1 mg/ml) | | | | | |
| Coverslip | 100 | $171 | 6 samples/ | 600 | $0.29 |
| (Ibidi) | | | slide | | |
| Sticky | 15 Slides, | $261 | 1 ch/ | 90 | $2.88 |
| Slide | 6 Channel/ | | sample | | |
| | Slide | | | | |
| mPEG | 5 g | $504 | 50 mg/20 | 12000 | $0.042 |
| NHS | | | slides | | |
| Biotin- | 1 g | $504 | 0.5 mg/20 | 240000 | $0.0021 |
| PEG- | | | slides | | |
| SVA | | | | | |
| | | | | Total price | $3.22 |
| | | | | per sample | |

TABLE 7
Fluorophores used for PSFs simulations

| Name | AF 488 | ATTO520 | AF 514 | 6-JOE | Cy3 | AF 546 | ATTO550 | AF 568 |
|---|---|---|---|---|---|---|---|---|
| Ex. max (nm) | 493 | 496 | 517 | 519 | 520 | 554 | 554 | 561 |
| Em. | 517 | 538 | 543 | 548 | 566 | 572 | 577 | 603 |
| Max (nm) | | | | | | | | |
| Ex. Factor | 0.94 | 0.42 | 0.43 | 0.38 | 0.93 | 1.09 | 0.90 | 0.69 |
| Name | AF 594 | ATTO490LS | AF 647 | AF 660 | AF 680 | ATTO700 | AF 700 | AF 750 |
| Ex. max (nm) | 579 | 590 | 653 | 663 | 679 | 696 | 700 | 752 |
| Em. | 618 | 658 | 670 | 691 | 702 | 719 | 719 | 779 |
| Max (nm) | | | | | | | | |
| Ex. Factor | 0.54 | 1.04 | 0.79 | 1.15 | 0.74 | 0.44 | 0.56 | 0.22 |

The fluorophores' peak excitation and emission wavelengths are given together with their calculated excitation factor (sum of all excitation spectrum values at 488, 561, and 640 nm laser lines)

TABLE 8
N2V configuration parameters (N2V Version: 0.3.2)

| N2V Parameter | Value |
|---|---|
| unet_kern_size | 3 |
| train_loss | mae |
| batch_norm | True |
| train_batch_size | 128 |
| n2v_perc_pix | 0.198 |
| n2v_patch_shape | (64,64) |
| n2v_neighborhood_radius | 3 |
| unet_residual | False |
| n2v_manipulator | median |
| blurpool | True |
| skipone | False |
| train_steps_per_epoch | N_{patches}/128 |

250 randomly selected fields-of-view (FOVs), from an artificial mix of all the single species datasets (with equal proportions to each miR dataset) were selected. From these images, the N2V method of generate_patches_from_list generated patches to train the model. The model was trained for 100 epochs, although about 20 epochs also seemed to suffice

TABLE 9
ThunderSTORM configuration parameters (TS version: 1.3)

| ThunderSTORM | | |
|---|---|---|
| category | Parameter | Value |
| Camera setup | Pixel size [nm] | 120 |
| | Photoelectrons per A/D | 3.6 |
| | count | |
| | Base level [A/D counts] | 0 |
| | EM gain | Unchecked |
| Image filtering | Filter | Wavelet filter |
| | | (B-Spline) |
| | B-Spline order | 3 |
| | B-Spline scale | 1.7 |
| Approximate | Method | Local maximum |
| localization of | Peak intensity threshold | 20* |
| molecules | Connectivity | 8-neighborhood |
| Sub-pixel | Method | PSF: Integrated |
| localization of | | Gaussian |
| molecules | Fitting radius [px] | 3 |
| | Fitting method | Weighted Least |
| | | Squares |
| | Initial sigma [px] | 1.6 |
| | Multi-emitter fitting | Disabled |
| | analysis | |

This parameter can change for different experiments according to the sample-specific SNR and noise distribution. The peak intensity threshold was selected by visual trial and error to ensure all miRs are detected, even at the cost of adding many false detections (due to the classifier's ability to remove them in the next processing steps, see methods)

TABLE 10
Classification pipeline parameters (sklearn version 1.1.3)

| | | |
|---|---|---|
| Augmentation | Number of | X6 the number of visually-labeled |
| | augmented crops | denoised miR crops (omitting |
| | | crops labeled as noise), added to |
| | | the original crops |
| | Augmentation | Gaussian noise with mean zero |
| | strength | and standard deviation of 15 |
| Normalization | Normalization | Normalize the entire crop so the |
| | type | entries of pixels (7,5), (7,6), |
| | | (8,5), (8,6) have a mean of 1 |
| | | (counting pixels from 1). |
| PCA | Number of | 20 |
| | components | |
| SVM | Kernel | RBF |
| | Inverse | 0.05 |
| | regularization C | |

SVM was trained using sklearn.svm.SVC and PCA was trained using sklearn.decomposition.PCA.

### REFERENCES FOR EXAMPLE 3

(1) Ha, M.; Kim, V. N. Regulation of MicroRNA Biogenesis. Nature Reviews Molecular Cell Biology 2014, 15 (8), 509-524. www(dot)doi(dot)org/10.1038/nrm3838.

(2) Bartel, D. P. Metazoan MicroRNAs. Cell 2018, 173 (1), 20-51. www(dot)doi(dot)org/10.1016/J.CELL.2018.03.006.
(3) Friedman, R. C.; Farh, K. K. H.; Burge, C. B.; Bartel, D. P. Most Mammalian MRNAs Are Conserved Targets of MicroRNAs. Genome Research 2009, 19 (1), 92-105. www(dot)doi(dot)org/10.1101/GR.082701.108.
(4) Lujambio, A.; Lowe, S. W. The Microcosmos of Cancer. Nature 2012, 482 (7385), 347-355. www(dot)doi(dot)org/10.1038/nature10888.
(5) Cortez, M. A.; Bueso-Ramos, C.; Ferdin, J.; Lopez-Berestein, G.; Sood, A. K.; Calin, G. A. MicroRNAs in Body Fluids—the Mix of Hormones and Biomarkers. Nature Reviews Clinical Oncology 2011, 8 (8), 467-477. www(dot)doi(dot)org/10.1038/nrclinonc.2011.76.
(6) Goodall, G. J.; Wickramasinghe, V. O. RNA in Cancer. Nature Reviews Cancer 2020 21:1 2020, 21 (1), 22-36. www(dot)doi(dot)org/10.1038/s41568-020-00306-0.
(7) Wu, Y.; Li, Q.; Zhang, R.; Dai, X.; Chen, W.; Xing, D. Circulating MicroRNAs: Biomarkers of Disease. Clinica Chimica Acta 2021, 516, 46-54. www(dot)doi(dot)org/10.1016/J.CCA.2021.01.008.
(8) Balatti, V.; Pekarky, Y.; Croce, C. M. Role of MicroRNA in Chronic Lymphocytic Leukemia Onset and Progression. Journal of Hematology and Oncology 2015, 8 (1), 1-6. www(dot)doi(dot)org/10.1186/S13045-015-0112-X/FIGURES/1.
(9) Drees, E. E. E.; Pegtel, D. M. Circulating MiRNAs as Biomarkers in Aggressive B Cell Lymphomas. Trends in cancer 2020, 6 (11), 910-923. www(dot)doi(dot)org/10.1016/j.trecan.2020.06.003.
(10) Schwarzenbach, H.; Hoon, D. S. B.; Pantel, K. Cell-Free Nucleic Acids as Biomarkers in Cancer Patients. Nature Reviews Cancer 2011 11:6 2011, 11 (6), 426-437. www(dot)doi(dot)org/10.1038/nrc3066.
(11) Elias, K. M.; Fendler, W.; Stawiski, K.; Fiascone, S. J.; Vitonis, A. F.; Berkowitz, R. S.; Frendl, G.; Konstantinopoulos, P.; Crum, C. P.; Kedzierska, M.; Cramer, D. W.; Chowdhury, D. Diagnostic Potential for a Serum MiRNA Neural Network for Detection of Ovarian Cancer. eLife 2017, 6. www(dot)doi(dot)org/10.7554/eLife.28932.
(12) Moretti, F.; D'Antona, P.; Finardi, E.; Barbetta, M.; Dominioni, L.; Poli, A.; Gini, E.; Noonan, D. M.; Imperatori, A.; Rotolo, N.; Cattoni, M.; Campomenosi, P. Systematic Review and Critique of Circulating MiRNAs as Biomarkers of Stage I-II Non-Small Cell Lung Cancer. Oncotarget 2017, 8 (55), 94980-94996. www(dot)doi(dot)org/10.18632/oncotarget.21739.
(13) Jang, J. Y.; Kim, Y. S.; Kang, K. N.; Kim, K. H.; Park, Y. J.; Kim, C. W. Multiple MicroRNAs as Biomarkers for Early Breast Cancer Diagnosis. Molecular and Clinical Oncology 2021, 14 (2), 1-9. www(dot)doi(dot)org/10.3892/mco.2020.2193.
(14) Sarver, A. L.; Sarver, A. E.; Yuan, C.; Subramanian, S. OMCD: OncomiR Cancer Database. BMC Cancer 2018, 18 (1), 1-6. www(dot)doi(dot)org/10.1186/s12885-018-5085-z.
(15) Yang, Z.; Wu, L.; Wang, A.; Tang, W.; Zhao, Y.; Zhao, H.; Teschendorff, A. E. DbDEMC 2.0: Updated Database of Differentially Expressed MiRNAs in Human Cancers. Nucleic Acids Research 2017, 45 (D1), D812-D818. www(dot)doi(dot)org/10.1093/nar/gkw 1079.
(16) Huang, Z.; Shi, J.; Gao, Y.; Cui, C.; Zhang, S.; Li, J.; Zhou, Y.; Cui, Q. HMDD v3.0: A Database for Experimentally Supported Human MicroRNA-Disease Associations. Nucleic Acids Research 2019, 47 (D1), D1013-D1017. www(dot)doi(dot)org/10.1093/nar/gky1010.
(17) Dubey, S. R.; Ashavaid, T. F.; Abraham, P.; Paradkar, M. U. Factors Influencing Circulating MicroRNAs as Biomarkers for Liver Diseases. Molecular Biology Reports 2022, 49 (6), 4999-5016. www(dot)doi(dot)org/10.1007/s11033-022-07170-1.
(18) Ono, S.; Lam, S.; Nagahara, M.; Hoon, D. Circulating MicroRNA Biomarkers as Liquid Biopsy for Cancer Patients: Pros and Cons of Current Assays. Journal of Clinical Medicine 2015, 4 (10), 1890-1907. www(dot)doi(dot)org/10.3390/Jcm4101890.
(19) Cheng, Y.; Dong, L.; Zhang, J.; Zhao, Y.; Li, Z. Recent Advances in MicroRNA Detection. Analyst 2018, 143 (8), 1758-1774. www(dot)doi(dot)org/10.1039/C7ANO2001E.
(20) Precazzini, F.; Detassis, S.; Imperatori, A. S.; Denti, M. A.; Campomenosi, P. Measurements Methods for the Development of MicroRNA-Based Tests for Cancer Diagnosis. Int J Mol Sci 2021, 22 (3), 1176. www(dot)doi(dot)org/10.3390/ijms22031176.
(21) Kim, D. J.; Linnstaedt, S.; Palma, J.; Park, J. C.; Ntrivalas, E.; Kwak-Kim, J. Y. H.; Gilman-Sachs, A.; Beaman, K.; Hastings, M. L.; Martin, J. N.; Duelli, D. M. Plasma Components Affect Accuracy of Circulating Cancer-Related MicroRNA Quantitation. Journal of Molecular Diagnostics 2012, 14 (1), 71-80. www(dot)doi(dot)org/10.1016/j.jmoldx.2011.09.002.
(22) Pritchard, C. C.; Cheng, H. H.; Tewari, M. MicroRNA Profiling: Approaches and Considerations. Nature Reviews Genetics 2012 13:5 2012, 13 (5), 358-369. www(dot)doi(dot)org/10.1038/nrg3198.
(23) Taylor, S. C.; Nadeau, K.; Abbasi, M.; Lachance, C.; Nguyen, M.; Fenrich, J. The Ultimate QPCR Experiment: Producing Publication Quality, Reproducible Data the First Time. Trends in Biotechnology 2019, 37 (7), 761-774. www(dot)doi(dot)org/10.1016/J.TIBTECH.2018.12.002.
(24) Godoy, P. M.; Bhakta, N. R.; Barczak, A. J.; Cakmak, H.; Fisher, S.; MacKenzie, T. C.; Patel, T.; Price, R. W.; Smith, J. F.; Woodruff, P. G.; Erle, D. J. Large Differences in Small RNA Composition Between Human Biofluids. Cell Reports 2018, 25 (5), 1346-1358. www(dot)doi(dot)org/10.1016/j.celrep.2018.10.014.
(25) Hong, L. Z.; Zhou, L.; Zou, R.; Khoo, C. M.; Chew, A. L. S.; Chin, C.-L.; Shih, S.-J. Systematic Evaluation of Multiple QPCR Platforms, NanoString and MiRNA-Seq for MicroRNA Biomarker Discovery in Human Biofluids. Scientific reports 2021, 11 (1), 4435. www(dot)doi(dot)org/10.1038/s41598-021-83365-z.
(26) Mestdagh, P.; Hartmann, N.; Baeriswyl, L.; Andreasen, D.; Bernard, N.; Chen, C.; Cheo, D.; D'Andrade, P.; DeMayo, M.; Dennis, L.; Derveaux, S.; Feng, Y.; Fulmer-Smentek, S.; Gerstmayer, B.; Gouffon, J.; Grimley, C.; Lader, E.; Lee, K. Y.; Luo, S.; Mouritzen, P.; Narayanan, A.; Patel, S.; Peiffer, S.; Rüberg, S.; Schroth, G.; Schuster, D.; Shaffer, J. M.; Shelton, E. J.; Silveria, S.; Ulmanella, U.; Veeramachaneni, V.; Staedtler, F.; Peters, T.; Guettouche, T.; Wong, L.; Vandesompele, J. Evaluation of Quantitative MiRNA Expression Platforms in the MicroRNA Quality Control (MiRQC) Study. Nature methods 2014, 11 (8), 809-815. www(dot)doi(dot)org/10.1038/nmeth.3014.
(27) Prokopec, S. D.; Watson, J. D.; Waggott, D. M.; Smith, A. B.; Wu, A. H.; Okey, A. B.; Pohjanvirta, R.; Boutros, P. C. Systematic Evaluation of Medium-Throughput MRNA Abundance Platforms. RNA 2013, 19 (1), 51-62. www(dot)doi(dot)rg/10.1261/rna.034710.112.
(28) Geiss, G. K.; Bumgarner, R. E.; Birditt, B.; Dahl, T.; Dowidar, N.; Dunaway, D. L.; Fell, H. P.; Ferree, S.; George, R. D.; Grogan, T.; James, J. J.; Maysuria, M.; Mitton, J. D.; Oliveri, P.; Osborn, J. L.; Peng, T.; Ratcliffe, A. L.; Webster, P. J.; Davidson, E. H.; Hood, L. Direct Multiplexed Measurement of Gene Expression with Color-Coded Probe Pairs. Nature Biotechnology 2008, 26 (3), 317-325. www(dot)doi(dot)org/10.1038/nbt1385.
(29) Narrandes, S.; Xu, W. Gene Expression Detection Assay for Cancer Clinical Use. Journal of Cancer 2018, 9 (13), 2249-2265. www(dot)doi(dot)org/10.7150/jca.24744.
(30) Neely, L. A.; Patel, S.; Garver, J.; Gallo, M.; Hackett, M.; McLaughlin, S.; Nadel, M.; Harris, J.; Gullans, S.; Rooke, J. A Single-Molecule Method for the Quantitation of MicroRNA Gene Expression. Nature Methods 2006, 3 (1), 41-46. www(dot)doi(dot)org/10.1038/nmeth825.
(31) Zhang, H.; Huang, X.; Liu, J.; Liu, B. Simultaneous and Ultrasensitive Detection of Multiple MicroRNAs by Single-Molecule Fluorescence Imaging. Chemical Science 2020, 11 (15), 3812-3819. www(dot)doi(dot)org/10.1039/DOSC00580K.
(32) Liu, Y.; Li, B.; Wang, Y.-J.; Fan, Z.; Du, Y.; Li, B.; Liu, Y.-J.; Liu, B. In Situ Single-Molecule Imaging of MicroRNAs in Switchable Migrating Cells under Biomimetic Confinement. Anal. Chem. 2022, 94 (9), 4030-4038. www(dot)doi(dot)org/10.1021/acs.analchem.1c05223.
(33) Li, B.; Liu, Y.; Liu, Y.; Tian, T.; Yang, B.; Huang, X.; Liu, J.; Liu, B. Construction of Dual-Color Probes with Target-Triggered Signal Amplification for In Situ Single-Molecule Imaging of MicroRNA. ACS Nano 2020, 14 (7), 8116-8125. www(dot)doi(dot)org/10.1021/acsnano.0c01061.
(34) Boguslawski, S. J.; Smith, D. E.; Michalak, M. A.; Mickelson, K. E.; Yehle, C. O.; Patterson, W. L.; Carrico, R. J. Characterization of Monoclonal Antibody to DNA.RNA and Its Application to Immunodetection of Hybrids. Journal of immunological methods 1986, 89 (1), 123-130. www(dot)doi(dot)org/10.1016/0022-1759(86)90040-2.
(35) Jeffet, J.; Ionescu, A.; Michaeli, Y.; Torchinsky, D.; Perlson, E.; Craggs, T. D.; Ebenstein, Y. Multimodal Single-Molecule Microscopy with Continuously Controlled Spectral Resolution. Biophysical Reports 2021, 1 (1), 100013. www(dot)doi(dot)org/10.1016/j.bpr.2021.100013.
(36) Max, K. E. A.; Bertram, K.; Akat, K. M.; Bogardus, K. A.; Li, J.; Morozov, P.; Ben-Dov, I. Z.; Li, X.; Weiss, Z. R.; Azizian, A.; Sopeyin, A.; Diacovo, T. G.; Adamidi, C.; Williams, Z.; Tuschl, T. Human Plasma and Serum Extracellular Small RNA Reference Profiles and Their Clinical Utility. Proceedings of the National Academy of Sciences 2018, 115 (23), E5334-E5343. www(dot)doi(dot)org/10.1073/pnas.1714397115.
(37) Krull, A.; Buchholz, T. O.; Jug, F. Noise2void-Learning Denoising from Single Noisy Images. Proceedings of the IEEE Computer Society Conference on Computer Vision and Pattern Recognition 2019, 2019-June, 2124-2132. www(dot)doi(dot)org/10.1109/CVPR.2019.00223.
(38) Ovesný, M.; Křížek, P.; Borkovec, J.; Švindrych, Z.; Hagen, G. M. ThunderSTORM: A Comprehensive ImageJ Plug-in for PALM and STORM Data Analysis and Super-Resolution Imaging. Bioinformatics 2014, 30 (16), 2389-2390. www(dot)doi(dot)org/10.1093/bioinformatics/btu202.
(39) Precision-Recall. scikit-learn. www(dot)scikit-learn/stable/auto_examples/model_selection/plot_precision_recall.html (accessed 2023-05-28).
(40) Shechtman, Y. Recent Advances in Point Spread Function Engineering and Related Computational Microscopy Approaches: From One Viewpoint. Biophys Rev 2020, 12 (6), 1303-1309. www(dot)doi(dot)org/10.1007/s12551-020-00773-7.
(41) Douglass, K. M.; Sieben, C.; Archetti, A.; Lambert, A.; Manley, S. Super-Resolution Imaging of Multiple Cells by Optimized Flat-Field Epi-Illumination. Nature Photonics 2016, 10 (11), 705-708. www(dot)doi(dot)org/10.1038/nphoton.2016.200.
(42) Edelstein, A. D.; Tsuchida, M. A.; Amodaj, N.; Pinkard, H.; Vale, R. D.; Stuurman, N. Advanced Methods of Microscope Control Using MManager Software. Journal of Biological Methods 2014, 1 (2), 10. www(dot)doi(dot)org/10.14440/jbm.2014.36.
(43) Scikit Support Vector Machines—Kernels. scikit-learn. www(dot)cikit-learn/stable/modules/svm.html (accessed 2023-05-29).
(44) Bishop, C. M. Pattern Recognition and Machine Learning; Information science and statistics; Springer: New York, 2006.
(45) scikit-learn—predict classification. www(dot)scikit-learn(dot)org/stable/modules/svm.html#classification.

(46) Scikit—Support Vector Machines—Scores and probabilities. scikit-learn. www(dot)scikit-learn/stable/modules/svm.html#scores-probabilities (accessed 2023-05-28).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of identifying a plurality of molecular species in a sample, the method comprising:
for each of at least two of the molecular species, respectively generating at least two uniquely labeled molecular species, each comprising, one of said species and, at respective two locations, two fluorophores having different emission wavelengths, wherein a combination of said fluorophores and/or a spatial distance between said fluorophores is unique for each of said at least two molecular species;
dispersing light emitted from said fluorophores onto an imager to generate an image of fluorophore emissions from said at least two uniquely labeled molecular species, wherein for any pair of emission wavelengths, a distance between fluorophore emissions corresponding to said pair is unique among all other pairs; and
correlating distances between fluorophore emissions in said image to said at least two of the molecular species in the sample, thereby identifying a plurality of molecular species in the sample.

2. A method of diagnosing a disease of a subject comprising identifying a plurality of molecular species in a sample of the subject, the method comprising:
for each of at least two of the molecular species, respectively generating at least two uniquely labeled molecular species, each comprising, one of said species and, at respective two locations, two fluorophores having different emission wavelengths, wherein a combination of said fluorophores and/or a spatial distance between said fluorophores is unique for each of said at least two molecular species;
dispersing light emitted from said fluorophores onto an imager to generate an image of fluorophore emissions from said at least two uniquely labeled molecular species, wherein for any pair of emission wavelengths, a distance between fluorophore emissions corresponding to said pair is unique among all other pairs; and
identifying said at least two of the molecular species by correlating distances between fluorophore emissions in said image to said at least two of the molecular species in the sample,
wherein a presence and/or an amount of said at least two molecular species is indicative of the disease.

3. The method of claim 1, wherein said combination of said fluorophores is unique among all other fluorophore-labeled molecular species in the sample.

4. The method according to claim 1, wherein said dispersing light from said fluorophores onto said imager generates a single image of fluorophore emissions from said at least two uniquely labeled molecular species.

5. (canceled)

6. (canceled)

7. The method according to claim 1, wherein said fluorophores are arranged along a direction over said molecular species, and wherein said dispersing is one-dimensional along said direction.

8. The method according to claim 1, wherein said fluorophores are arranged along a first direction over said molecular species, and wherein said dispersing is one-dimensional along a second direction, different from said first direction.

9. (canceled)

10. The method according to claim 1, wherein said dispersing is two-dimensional.

11. The method according to claim 1, wherein at least one molecular species is labeled by at least three spaced apart locations on said species with respective at least three different fluorophores characterized by different emission wavelengths.

12. The method according to claim 1, wherein said image is a spectral image.

13. The method according to claim 1, wherein said dispersing comprises non-linear dispersing.

14. The method according to claim 1, wherein said dispersing comprises linear dispersing.

15. (canceled)

16. The method according to claim 1, wherein a combination of said fluorophores is unique for each of said at least two molecular species to generate at least two uniquely labeled molecular species.

17. (canceled)

18. The method according to claim 1, wherein a distance between said fluorophores is unique for each of said at least two molecular species to generate at least two uniquely labeled molecular species.

19. (canceled)

20. The method according to claim 1, wherein said imager is a component in an imaging system characterized by a diffraction limit, wherein a largest among spatial separations between said locations is less than said diffraction limit, and wherein a smallest among said distances between fluorophore emissions is larger than said diffraction limit.

21. The method according to claim 1, further comprising analyzing intensity distribution of said fluorophore emissions in said image and correlating said intensity distribution to said at least two of the molecular species in the sample.

22. The method according to claim 1, further comprising orientating said at least two uniquely labeled molecular species with respect to one another.

23. The method according to claim 1, further comprising orientating said at least two uniquely labeled molecular species along a direction.

24. The method according to claim 1, further comprising immobilizing said at least two uniquely labeled molecular species on a solid surface.

25-27. (canceled)

28. The method according to claim 1, wherein said molecular species are selected from the group consisting of a polypeptide, a polynucleotide, a lipid and a carbohydrate.

29-40. (canceled)

41. A method of identifying a plurality of molecular species in a sample, the method comprising:
for each of the molecular species, respectively generating at least two uniquely labeled molecular species, each comprising, one of said species and, at a plurality of locations along a first direction, a sequence of fluorophores, wherein a spatial distribution of said fluorophores along said first direction is unique for each of said at least two molecular species to generate at least two uniquely labeled molecular species;
dispersing light emitted from said fluorophores along a second direction onto an imager to generate an image of fluorophore emissions from said at least two uniquely labeled molecular species, wherein said first direction is different from said second direction;
identifying different two-dimensional patterns of fluorophore emissions in said image; and
correlating said identified patterns to at least two of the molecular species in the sample, thereby identifying a plurality of molecular species in the sample.

42-50. (canceled)
